(19) United States
(12) Patent Application Publication
(10) Pub. No.: US 2026/0033936 A1
(43) Pub. Date: Feb. 5, 2026

(54) METHODS OF MEASURING FLUORESCENCE AND CAPTURING MOVEMENT IN ANIMALS

(71) Applicants: Georgia Tech Research Corporation, Atlanta, GA (US); Emory University, Atlanta, GA (US)

(72) Inventors: Jeffrey Markowitz, Atlanta, GA (US); Emine Zeynep Ulutas, Atlanta, GA (US); Amartya Pradhan, Atlanta, GA (US); Dorothy Koveal, Atlanta, GA (US)

(21) Appl. No.: 19/288,867

(22) Filed: Aug. 1, 2025

Related U.S. Application Data
(60) Provisional application No. 63/678,321, filed on Aug. 1, 2024.

(51) Int. Cl. A61D 99/00; A61K 51/12

(52) U.S. Cl. CPC A61D 99/00; A61K 51/1251

(57) ABSTRACT

The present application is directed towards a method of tracking fluorescence in animals using fluorescent particles.

### CROSS-REFERENCE TO RELATED APPLICATION

This application claims the benefit of U.S. Provisional Application No. 63/678,321 filed on Aug. 1, 2024, the entire contents of which are incorporated by reference herein.

### BACKGROUND OF THE INVENTION

Animal movement has traditionally been characterized by manually scoring video footage or direct observation (1-4). Recent technological advances in machine learning have enabled the study of unrestrained, naturalistic movements with unprecedented speed and accuracy, especially in the field of markerless keypoint tracking, which has been transformative for neuroscience (5-10). State-of-the-art methods such as transfer learning with convolutional networks (9), U-Net-inspired architectures (10, 11), and 3D convolution networks (6) have enabled tracking of discrete computationally-defined points on the surface of laboratory animals trained on a small number of hand-labeled video frames. These advances, in turn, have enabled new methods for classifying animal behaviors (12-14) and quantifying movement (15, 16).

However, due to variations in experimental setups across laboratories, markerless keypoint trackers still require hand-labeling datasets. That can introduce jitter due to variation between annotators and the inherent ambiguity of labeling certain body parts based solely on surface features, such as positions along the back of a mouse. In rats, where markerless keypoint trackers have been systematically compared against skin-attached fiducials, their predictions were estimated to have precision on the order of ±10 mm (17), comparable to the distance between many key landmarks on the mouse (18). That contrasts with commercial motion capture systems used with humans, which have a demonstrated precision of approximately ±0.1 mm (19-21)

Another limitation of markerless keypoint trackers is that they are trained to identify points on the outside of the animal's body using surface features visible in videography. As joint and skeletal kinematics can be obscured by soft tissue and fur in rodents (22, 23) and humans (24, 25), it remains unclear if movement of the skeleton can be resolved in this way. That is important because the brain directly controls the muscles, which exert complex forces on the skeleton. The joints exert important constraints on skeletal motion and thus shape resulting animal movements (26). It remains unclear if tracking motions on the surface of the skin is a viable strategy for resolving the brain's control of movement and how it breaks down in disease and injury (22, 25, 27-29).

Though technically demanding, it is possible to directly observe the skeletal system in live rats using X-ray videography, which has demonstrated the principle that skin-derived joint angles and kinematics can greatly diverge from those derived directly from the skeleton (22). While this technique has very high spatial resolution, X-ray videography is challenging to set up in individual labs and can be limited by radiation dosage, allowing only brief imaging sessions (30-32). Imaging over long periods of time is important for longitudinal experiments, such as observing the development of movement or disease progression.

Despite the considerable progress in the development and deployment of markerless keypoint trackers, these approaches are limited in their ability to measure the part of the body directly controlled by the brain—the musculoskeletal system. Markerless keypoint trackers are trained to identify points on the outside of the animal's body using surface features visible in videography. It remains unclear if movement of the skeleton can be resolved in this way as musculoskeletal dynamics are obscured by soft tissue and fur in rodents (Bauman & Chang, 2010; Filipe et al., 2006; Monsees et al., 2022). Direct observation of the skeleton via X-ray presents other challenges, including a limited imaging volume and radiation dosage (Bonnan et al., 2016; Brainerd et al., 2010; Monsees et al., 2022; Witte et al., 2002).

An alternate strategy would be to implant optical tags in key landmarks inside the body, including the joints, that can be measured non-invasively. To be successful, this approach must satisfy two important criteria. First, the implanted optical tag must be detectable from outside of the animal. Prior studies suggest that near-infrared I (NIR-I, 650-900 nm) is an ideal spectral window for non-invasive imaging due to minimal light absorption and scattering by skin (33-35) and hemoglobin (36). Quantum dots (QDs) are one of the few fluorescent materials in this spectral range that are photostable (i.e., minimal photobleaching) and bright (i.e., high quantum yields and extinction coefficients), making them an attractive material relative to NIR-fluorescent proteins and dyes (34, 37-39). Second, the implanted optical tag should be long-lasting to support long-term studies of movement in an animal. In addition to being photostable, it is therefore important for the tag to be biocompatible and to have a long half-life in vivo. quantum dots are widely used in the life sciences and thus are readily available in numerous biocompatible formulations (39-42) and have been used in vivo (43, 44). However, the in vivo half-life of different quantum dot formulations has not previously been investigated.

### SUMMARY OF THE INVENTION

In one aspect, the present invention is directed to a method of measuring fluorescent particles within animals. In some embodiments of the present invention, the animal is injected with fluorescent particles, and the fluorescence is captured. In some instances, the fluorescent particle is bound to a microparticle. In some instances, the microparticle is a polymethyl-methacrylate (PMMA) bead. In some instances, the microparticle is an agarose bead. In some instances, the fluorescent particle is coated in biotin. In some instances, the microparticle is streptavidin. In some instances, the fluorescent particle is coated in biotin and binds to a microparticle that is streptavidin.

Some embodiments of the present invention are directed towards a method of measuring fluorescence in an animal, the method comprising (a) injecting the animal with one or more fluorescent particles; and (b) capturing the fluorescence exhibited by the animal; wherein the fluorescent particle is bound to a microparticle.

In some embodiments, the fluorescent particle is a quantum dot.

In some embodiments, the microparticle is a polymethyl-methacrylate (PMMA) bead, an agarose bead, or other microparticle coated with an antibody.

In some embodiments, the fluorescent particle is coated in biotin, and the microparticle is streptavidin.

In some embodiments, the antibody is an antibody-binding protein to collagen.

In some embodiments, the antibody is an antibody-binding protein to fibronectin.

In some embodiments, the quantum dot emits at an excitation from about 600 nm to about 1000 nm.

In some embodiments, the animal is a mammal.

In some embodiments, the mammal is a mouse.

In some embodiments, the mouse is injected in at least one of the right paw, the left paw, the right hind leg, the left hind leg, the tail, subdermal, the spine, joints (e.g. knee, wrist), the ears, the head and other internal organs (e.g. the bladder).

In some embodiments, a camera captures the fluorescence.

In some embodiments, the camera is a near infrared camera.

In some embodiments, the animal is a diseased animal, an animal that has been treated (e.g. with a drug), or a genetically-modified animal.

In some embodiments, the disease is a neurodegenerative disease.

In some embodiments, the neurodegenerative disease is Parkinson's or Huntington's.

Some embodiments of the present invention are directed towards a method of measuring movement of an animal, the method comprising: (a) injecting the animal with one or more fluorescent particle(s) bound to a microparticle; (b) capturing the fluorescence exhibited by the animal; and (c) monitoring the fluorescence to measure the movement of the animal.

In some embodiments, the movement of the animal is measured using a camera.

In some embodiments, the animal has a neurodegenerative disorder.

In some embodiments, the method further comprises comparing the movement of the animal to a set of criteria to determine if the animal suffers from a neurodegenerative disorder; wherein the criteria is if the animal has at least one of a halting gait, a tremor, dyskinesia, tics, or chorea.

In some embodiments, the microparticle is a quantum dot.

In some embodiments of the present invention, the fluorescent particle is a quantum dot.

In some embodiments of the present invention, the quantum dot emits at an excitation wavelength from about 700 nm to about 850 nm.

In some embodiments of the present invention, the animal (such as a mouse) is injected in at least one of the right paw, the left paw, the right hind leg, the left hind leg, the tail, intramuscular, intradermal, intra-articular, subdermal, or the spine.

In some embodiments of the present invention, a camera captures the fluorescence. In some instances, the camera is an infrared camera.

In some embodiments of the present invention, the animal is an animal that has been treated (e.g. with a drug). In some instances, the animal is a genetically-modified animal. In some instances, the genetically modified animal is a transgenic animal. In some instances, a gene is knocked out in the animal. In some instances, a gene is inserted into the animal. In some instances, the transgenic animal is a conditional model.

In some embodiments of the present invention, the animal is a diseased animal. In some instances, the disease is a neurodegenerative disease. In some instances, the neurodegenerative disease is Parkinson's disease or Huntington's disease. In some instances, the disease is cancer. In some instances, the cancer is a blood cancer. In some instances, the cancer is a tumor.

### BRIEF DESCRIPTION OF DRAWINGS

FIG. 1A is a schematic representation of the quantum dot injection procedure.

FIG. 1B is a schematic representation of the basic optical properties of NIR-emitting quantum dots.

FIG. 1C is a histological examination of quantum dots injected to the back of a mouse.

FIG. 1C, top, is a schematic of a quantum dot injection. FIG. 1C, bottom left, is fluorescence analysis of the mouse ex vivo. FIG. 1C, bottom right, is quantification of the fluorescence ex vivo.

FIG. 1D is a schematic of the plexiglass arena and optical configuration for in vivo imaging.

FIG. 1E is an example of images of reflectance images (top) and fluorescence images (bottom).

FIG. 1F is a schematic illustrating how quantum dot fluorescence is measured.

FIG. 1G is a graph of the 95^{th} percentile pixel percentile pixel intensity comparison of vehicle/blank (n=5 mice) and quantum dot mice (n=6 mice).

FIG. 1H is a graph of the average pixel intensity comparison between vehicle/blank or quantum dot injection.

FIG. 2A is a schematic of quantum injection sites with QD800.1 (top) and QD800.2 (bottom).

FIG. 2B is an image of the reflectance image (top) and fluorescence image (bottom) of a mouse immediately after injection of QD800.2.

FIG. 2C is example fluorescence images from QD800.1 (top) and QD 800.2 (bottom) imaged at 0 and 2 days post-injection.

FIG. 2D are examples of confocal images from injections into the backs of mice with either QD800.1 (top) or QD800.2 (bottom).

FIG. 2E is a graph of the ratio of quantum dot fluorescence inside to outside of cells.

FIG. 2F is a graph the initial brightness (top) and decay rate (bottom) for both variants calculated from 95^{th} percentile variants.

FIG. 2G is a graph Initial brightness (top) and the fluorescence longevity (time for the trace for each mouse/camera pair to cross below the 99^{th} percentile of vehicle/blank mice, bottom).

FIG. 2H is a schematic of spatial autocorrelation calculation to measure the length-scale of quantum-dot induced fluorescence computed using average pixel intensity over time.

FIG. 2I is a schematic of spatial autocorrelation calculation to measure the length-scale of quantum dot-induced fluorescence.

FIG. 2J is a graph of average in vivo spatial correlation of fluorescence across all mice and camera views for both QD800.1 (top) and QD800.2 (bottom)

FIG. 3A is a Schematic representation of quantum dot injection sites with QD800.3.

FIG. 3B is brightfield (left) and fluorescence (right) images from the same tissue sample taken from the back 1 day after QD800.3 injection. Scale bar represents 500 μm. FIG. 3B, top, is a schematic of injecting quantum dot coated agarose beads into a mouse.

FIG. 3C is Example fluorescence images from QD800.2 (top) and QD800.3 (bottom) imaged at 4 days post-injection. Scale bar indicates 50 pixels.

FIG. 3D is either 95^{th} percentile (top) or average (bottom) pixel intensities plotted as a function of days post-injection for all three variants. Line indicates the average and shaded region one standard deviation across mouse/camera pairs.

FIG. 3E is initial brightness (top) and the decay rate (bottom) for all variants computed using the 95^{th} percentile across time (p=0.013/0.19, U=173/144.5, f=0.77/0.64 for brightness; p=2.5E-6/2.3E-6, U=225/225, f=1/1 for longevity, Mann-Whitney U test; n=15 mouse/camera pairs each, QD800.3 compared with QD800.1/QD800.2).

FIG. 3F, similar to FIG. 3E, except computed using the average across time (p=0.004/0.009, U=182/176, f=0.81/0.78 for brightness; p=2.7-e6/2.5E-6, U=225/225, f=1/1 for longevity, Mann-Whitney U test; n=15 mouse/camera pairs each, QD800.3 compared with QD800.1/QD800.2).

FIG. 3G is graphs of spatial autocorrelation for QD800.1 (top left), QD800.2 (top right), and QD800.3 (bottom).

FIG. 4A is a schematic representation of quantum dot injection sites with variant 3.

FIG. 4B is fluorescence images from mice injected with QD800.3 using Rig v1 (top, Rig v1 is seen in FIG. 7) or Rig v2 (bottom, Rig v2 is seen in FIG. 13). Scale bar indicates 50 pixels.

FIG. 4C is a graph of signal to noise ratio across all variants and rigs, estimated by taking the standard deviation across x- and y-axes for each fluorescence frame and then averaging the result across all frames for each mouse/camera view pair.

FIG. 4D is a schematic illustrating scaling up of labeling.

FIG. 4E is a graph of keypoint prediction localization error relative to hand-labeled points using reflectance images (“reflectance only”), fluorescence images (“fluorescence only”), or their combination (“both”).

FIG. 4F is a graph of performance of the “both” model after subsampling the training dataset.

FIG. 4G are graphs of average held-out error (Euclidean distance between predicted keypoint and ground truth) for a model trained using a different random seed (n=5 restarts) with and without quantum dot fluorescence refinement across reflectance (top), fluorescence (middle), and reflectance+fluorescence (bottom).

FIG. 4H is a graph for each frame, the difference in pixel error is shown with and without fluorescence refinement across reflectance (top), fluorescence (middle), and reflectance+fluorescence (bottom).

FIG. 4I is a schematic for using machine-labeled frames to train markerless keypoint trackers on reflectance data only.

FIG. 4J is graphs of the U-Nets that were trained and tested using data from the same camera view (“same”, top), all camera views.

FIG. 4K is a graph of the results from training U-nets according to FIG. 4J.

FIG. 5A is a schematic of the strategy for conjugating QD800 to antibodies in order to target specific tissues, cell types, and molecules.

FIG. 5B are examples of fluorescent images of tissue was stained with DAPI (for nuclei) and collagen to assess localization, with the right image being a zoomed in version of the left image. The arrows are pointing to regions of overlap between DAPI, collagen, and QDs.

FIG. 5C is a graph of the ratio of quantum dot fluorescence colocalized with DAPI to collagen (n=13 fields of view from n=1 tissue sample/injection for QD800.4 and n=10 fields of view from n=1 tissue sample/injection for QD800.1).

FIG. 5D is a graph of the fluorescence plotted against days post injection with same plotting convention as FIGS. 2F and 3D. FIG. 5D, top, is the time 95^{th} percentile. FIG. 5D, bottom, is time average.

FIG. 5E is a graph of the quantification of longevity of fluorescence signal per mouse/camera pair using the 95^{th} percentile across time (p=1.5E-5, U-0, f-0 for longevity comparing QD800.3 to QD800.4.COLLAGEN, Mann-Whitney U test; p=1.5E-5, U=0, f-0 comparing QD800.3 to QD800.4.FIBRONECTIN; n=15 mouse/camera pairs for QD800.3, n=10 mouse/camera pairs for each QD800.4 variant, and n=10 vehicle-injected mouse/camera pairs) and using the average across time (p=1.3E-5, U-0, f-0 for longevity comparing QD800.3 to QD800.4.COLLAGEN, Mann-Whitney U test; p=1.3E-5, U=0, f-0 comparing QD800.3 to QD800.4.FIBRONECTIN). Note that longevity for QD800.4 was as long as they were imaged.

FIG. 5F is a graph of the spread of fluorescence in x- and y-axes for QD800.3 (left) and QD800.4 (right) over time, assessed using the spatial autocorrelation.

FIG. 6A is a schematic of QD800.3 being injected directly into the knee joints after shaving fur.

FIG. 6B is images of in vivo cadaver validation of intra-articular targeting across brightfield (left) and fluorescence (right).

FIG. 6C is a micro-computed tomography (microCT) image from the right knee joint of mouse cadaver. Fluorescence is overlaid (yellow spot) on top of a reconstructed mouse skeleton. Arrow indicates location of fluorescence spot.

FIG. 6D are images of frames (Left is frame 0, middle is frame 5, and right is frame 10) from video recorded 1 hour post-injection into live mice.

FIG. 6E is a graph using the same procedure as FIG. 4J, with trained keypoint models to track knee joint location based on surface features. Plotting conventions are the same as FIG. 4J.

FIG. 7A is an image showing the rig and plexiglass arena used for imaging quantum dots (QDs) in freely moving mice. All data included in FIGS. 1-3 and FIGS. 8-4 were collected using this setup.

FIG. 7B is a schematic (bottom) of the illumination sequence used for temporal-division multiplexing. Note that the fluorescence images (top) presented here are not background-subtracted.

FIG. 8A is a schematic to remove static elements from the scene (e.g., excitation LEDs), the background of the image was computed using a non-overlapping 1500-frame-long sliding window. All visualization and quantification of fluorescence data uses background-subtracted data.

FIG. 8B is a (left) schematic of the quantification of fluorescence intensity and longevity in FIGS. 1, 2, 3, 4, and 5 using peak fluorescence across the entire frame and subsequently computed either the 95^{th} percentile or the average across frames with the right being example images.

FIG. 8C are graphs of either the average (bottom) or the 95^{th} percentile (top) of max intensity computed using either the full frame or the mouse ROI.

FIG. 8D are graphs of the analysis in FIG. 1G-H repeated using the peak fluorescence computed with the mouse ROI rather than the full frame for the 95^{th} percentile (top) or time average (bottom).

FIGS. 8E, 8F, and 8G are graphs of the analysis in FIG. 2F-H repeated using the peak fluorescence computed with the mouse ROI rather than the full frame. FIG. 8E represents the time 95^{th} percentile (top) and time average (bottom). FIG. 8F represents the fluorescence on day 0 (top) and fluorescence longevity (bottom) for the time 95^{th} percentile. FIG. 8G represents the fluorescence on day 0 (top) and the fluorescence longevity (bottom) for the time average.

FIG. 9 are images of the reflectance (left) and fluorescence data (right) for four frames-timestamps are given at the top—from all five hardware-synchronized cameras. Data from a mouse injected with QD800.2

FIG. 10 are images of the same layout as FIG. 9, except a different session is shown. Data from a mouse injected with QD800.2. The mouse shown here is different from the mouse shown in FIG. 9 across reflectance (left) and fluorescence (right).

FIG. 11A are images as a droplet from six different microbead brands (Amid Biosciences, #SA-101-1; Bangs Laboratories, #CP01008; Resyn Biosciences, #MR-STM002; PolyAn, #105-21-020; Thermo Scientific, #20357; Vector Laboratories, #N-1000-002) mixed with biotinylated quantum dots imaged under brightfield (top) and fluorescence (bottom) using Cy7 excitation/emission (see Methods for details). Scale bar represents 500 μm.

FIG. 11B schematic of how quantum dots can be attached-attaching quantum dots to a relatively large, biocompatible, porous agarose beads will lead to stabilization of quantum dot fluorescence called QD800.3.

FIG. 11C is a schematic of the protocol for QD800.3. Streptavidin-coated agarose beads are mixed with biotinylated quantum dots and incubated for 1 hour at 40° C., mixed halfway through the incubation period. The supernatant is removed, the solution is washed 3 times with 1×PBS, and the beads are resuspended in 2% sodium alginate.

FIG. 11D are images of agarose beads (top) and the agarose bead quantum dot mixture (bottom). Images were taken under 405 nm illumination.

FIG. 11E are images of a drop of agarose beads (top) or the agarose bead quantum dot mixture (bottom) under brightfield illumination. Scale bar represents 500 μm.

FIG. 11F are fluorescence images from the same droplet of agarose beads (top) and the agarose quantum dot mixture (bottom) of FIG. 11E using Cy7 excitation/emission (see Methods for details). Scale bar represents 500 μm.

FIG. 11G are images of the pipette tips for quantum dot injections. Each minor division is 10 μm.

FIG. 12 are images of (Left), schematic of experiment. QD800.3 was injected into the left forepaw, then tissue was harvested 1-week post-injection and imaged. (Middle): brightfield image of injection site. (Right): fluorescence image of the injection site. Scale bar represents 500 μm.

FIG. 13A is an image of a setup for fluorescence imaging.

FIG. 13B are images of quantum dots were pipetted onto a slide and imaged in the plexiglass arena once every 30 seconds for 24 hours to measure photostability in rig version 2. The top image is peck intensity and the bottom image is the image similarity to the last 10 frames (mean absolute error).

FIG. 14 are images using the same layout as FIGS. 9 and 10. The mouse shown here was injected with QD800.3.

FIG. 15A is images using keypoint predictions from the “reflect+fluo” model used in FIG. 4E-G, taking a 30×30-pixel window around each predicted keypoint location, and averaged windows across all predictions for each body part.

FIG. 15B is a Histogram of the intensity data shown in FIG. 15A using radial bins in the back (left), paws (middle) and tail (right).

FIG. 15C is the quantification of the data shown in FIG. 15A with peak pixel intensity shown on the left and distance from the center to reach 50% of peak intensity on the right (halfway).

FIG. 15D is images of the performance of impact of fluorescence intensity and the spread of fluorescence on detection of the center of fluorescence across the minimum projection (left) and maximum projection (right).

FIG. 15E are images of a filled contour plot of the simulations shown in FIG. 15D across the minimum projection (left) and maximum projection (right).

FIG. 15F is boxplot of the spread of fluorescence for the x-axis spread (top) and the y-axis spread (bottom) after repeated injections of QD800.2.

FIG. 15G is boxplots of the aspect ratio (top) and the time average (bottom) of peak fluorescence intensity per injection volume. Gray region indicates 99^{th} percentile from vehicle injected mice (same mice as in FIG. 5D).

FIG. 16A is histograms of maximum pairwise distance between n=4 labelers who each annotated the same n=271 reflectance frames with the 10 keypoints shown in FIG. 4D, and an additional n=274 reflectance frames with the 2 knee joints. The areas measure were the back (top left), paws (top right), tail (bottom left), and the knee (bottom right)

FIG. 16B is (top), histograms of discrepancies across labelers for reflectance frames (blue), and the discrepancy between labelers and quantum dot fluorescence centroids with frames with alpha-blended fluorescence (orange). (Bottom), cumulative histograms of the same data shown on top.

FIG. 16C is boxplots of pairwise errors across labelers for reflectance frames along with the discrepancy between labelers and quantum dot fluorescence centroids for each of 6 different labelers.

FIG. 17A is heatmaps of (Left): median distance between predicted keypoints and fluorescence centroids shown as a function of distance from each camera and the viewing angle. (Right): median distance computed across all cameras.

FIG. 17B is a graph of the distance between keypoints and quantum dot fluorescence centroid shown as a function of position in the arena at each location. The left is the 25^{th} percentile (q=0.25) of the error surface, the middle is the 50^{th} percentile (q=0.5) of the surface, and the right is the 75^{th} percentile of the error surface (q=0.75).

FIG. 18A are heat maps for the “reflect+fluo” model from FIG. 4E, determined as the best set of parameters through a grid search of key U-Net parameters.

FIG. 18B are graphs of the dataset used to train models in FIG. 4K (QD-Pi-120k), the distance to the nearest neighbor computed for each keypoint, across the paws (top), back (middle), and tail (bottom).

FIG. 18C are graphs used to determine the percent of QD-Pi-120k frames in which the mouse is moving at various speeds, the velocity was calculated of the mouse's centroid using triangulated keypoints (see FIG. 17 and Methods for details) various thresholds between 25-200 mm/s and calculated the percent frames above each threshold.

FIG. 18D are graphs of the performance of the models shown in FIG. 4K broken down by body part for each condition of the same camera view as training data (left), all camera views used as training data (middle), and different camera views used as training data and tested on a held out view (right, 4 camera views were tested on a 5^{th} held out view). Conventions are the same as FIG. 4K.

FIG. 18E are graphs of different performance metrics for the models shown in FIG. 4K: precision (top), recall (middle), and object keypoint similarity (OKS) (bottom). Line reflects the median inverted power-law decay fit, and the shaded region indicates 95% bootstrap confidence interval.

FIG. 19A is a schematic of the illumination sequence with shorter exposure times. Note that here fluorescence images are not background subtracted.

FIG. 19B is box plots of (top): time average of peak fluorescence intensity from n=5 mice injected with QD800.2 imaged at different exposure times and with different analog gains on each camera. (middle): spread along the x-axis from mice imaged at different exposure times and gains measured using the standard deviation along the x-axis of the 2D autocorrelation function from the fluorescence channel. (bottom): spread along the y-axis, computed using the same method as for the x-axis.

### DETAILED DESCRIPTION OF THE INVENTION

The following description of the embodiments is merely exemplary in nature and is in no way intended to limit the subject matter of the present disclosure, their application, or uses.

As used throughout, ranges are used as shorthand for describing each and every value that is within the range. Any value within the range can be selected as the terminus of the range. Unless otherwise specified, all percentages and amounts expressed herein and elsewhere in the specification should be understood to refer to percentages by weight.

For the purposes of this specification and appended claims, unless otherwise indicated, all numbers expressing quantities, percentages or proportions, and other numerical values used in the specification and claims, are to be understood as being modified in all instances by the term “about.” The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent, alternatively±5 percent, alternatively±1 percent, alternatively±0.5 percent, and alternatively±0.1 percent of the given numeric value provided such a deviation does not alter the end function or result of the value. Accordingly, unless indicated to the contrary, the numerical parameters set forth in this specification and attached claims are approximations that can vary depending upon the desired properties sought to be obtained by the present invention.

As used in this specification and the appended claims, the singular forms “a,” “an,” and “the,” include plural references unless expressly and unequivocally limited to one referent. As used herein, the term “include” and its grammatical variants are intended to be non-limiting, such that recitation of items in a list is not to the exclusion of other like items that can be substituted or added to the listed items. For example, as used in this specification and the following claims, the terms “comprise” (as well as forms, derivatives, or variations thereof, such as “comprising” and “comprises”), “include” (as well as forms, derivatives, or variations thereof, such as “including” and “includes”) and “has” (as well as forms, derivatives, or variations thereof, such as “having” and “have”) are inclusive (i.e., open-ended) and do not exclude additional elements or steps. Accordingly, these terms are intended to not only cover the recited element(s) or step(s), but may also include other elements or steps not expressly recited. Furthermore, as used herein, the use of the terms “a” or “an” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

As used herein, the term “about” when appearing before a range should be understood as referring to both endpoints of the range. In such instances the range should also be understood as including the range defined by the specific endpoints listed, and also including sub-ranges within the listed endpoints. In the instances where “about” is appearing before a number it should be understood as the number includes the range of +/−5%.

In contrast with existing methods, described herein is a new high-precision method that captures the position of points inside a mouse's body. The presently described technique leverages fluorescent nanoparticle-derived markers embedded underneath the mouse's skin, either superficially near the surface, or deep inside the body (e.g. in joints), which can be subsequently imaged in freely moving animals using standard machine-vision cameras.

Various aspects of the disclosure pertain to a method of tracking fluorescent particles injected into an animal, such as, for example, a mouse. According to various aspects of the disclosure, the tracking fluorescent particles are nanoparticles, such as, for example, quantum dots.

Described herein, the inventors have invented a method called QD-Pi (Quantum Dot-based Pose estimation in vivo). Quantum Dot-based Pose uses near-infrared-I-emitting quantum dots as injectable, bright, long-lived optical probes that can be imaged non-invasively in freely moving animals using standard machine-vision cameras. The inventors further found that Quantum Dot-based Pose can be used to reliably track keypoint markers in the joints while mice freely move in an open plexiglass arena.

In accordance with various aspects of the present disclosure, nanoparticles are injected into animals, such as, for example, mice. In some instances, the nanoparticles are quantum dots. In some instances, the quantum dots are injected into various parts of the body, such as, for example, the right forepaw, the right hind leg, the left forepaw, the left hindleg, the tail, into fatty tissue, into organs, and along the spine. In some instances, the nano-particles are injected into the muscular joints. In some instances, the joint is a knee joint or a wrist joint. In some instances, the quantum dot is injected intraarticularly (within joints). In some instances, the nano-particles are injected into the head. In some instances, the quantum dots are injected into the internal organs, such as, for example, the bladder. In some instances, the nanoparticles are injected subdermal into the animal. In some instances, the quantum dot is about 0.01 to about 3.5 mm beneath the skin, alternatively from about 0.1 to about 3.5 mm beneath the skin, alternatively from about 0.1 to 1 mm beneath the skin, alternatively about 1 to about 3.5 mm beneath the skin, and alternatively about 1 to about 3 mm beneath the skin. In some instances, the quantum dot is injected as dorsal and ventral injection into each paw of the animal. In some instances, the volume of quantum dots injected into the mouse is in an amount ranging from about 0.5 μL to about 10 μL, alternatively about 0.5 μL to about 5 μL, and alternatively about 1 μL to about 4 μL.

In accordance with various aspects of the disclosure, quantum dots to be injected are fluorescent. In some instances, the excitation wavelength of the quantum dot ranges from about 600 nm to about 2500 nm, alternatively from about 680 nm to about 1500 nm, and alternatively from about 700 nm to about 760 nm. In some instances, the excitation wavelength of the quantum dot In some instances, when the excitation wavelength of the quantum dot dips below 600 nm, the excitation wavelength starts to fall into the visibility spectrum of the mouse. Therefore, the mouse will start to see itself fluoresce. In some instances, the quantum dot is visible on a near infrared-I spectrum, infrared spectrum, or into longer wavelengths such as the short-wave infrared range.

In accordance with various aspects of the disclosure, the quantum dot is coated. In some instances, the quantum dot is coated with an amphiphilic polymer coating. In some instances, the coating is a carboxyl-derivatized amphiphilic coating. In some instances, the coating can be coupled to a peptide. In some instance the coating can be coupled to the amine group of proteins. In some instances, the coating can be coupled to modified oligonucleotides. In some instances, the coating is an aliphatic hydrocarbon surface coating. In some instances, the coating is polyethylene glycol (PEG). In some instances, the coating is streptavidin. In some instances, the quantum dot is the Qtracker™ 800 Cell Labeling kit from Thermofisher. In some instances, the quantum dot is the Qtracker™ 800 Vascular label from Thermofisher. In some instances, the quantum dot is the Qdot™ 800 Streptavidin Conjugate from Thermofisher. In some instances, the quantum dot is a Qdot 800 Probe from Thermofisher. In some instances, the quantum dot is labelled with an antibody. In some instances, the quantum dot is labelled using the Qtracker™ 800 Cell Labeling Kit. In accordance with various aspects of the disclosure, the quantum dot labels the vascular. In some instances, the quantum dots are coated in a peptide that facilitate uptake into cells. In some instances, the peptide is polyarginine.

In accordance with various aspects of the disclosure, the quantum dot is bound to a microparticle. In some instances, the microparticle is polymethyl-methacrylate (PMMA) beads. In some instances, the fluorescent particle is coated in biotin. In some instances, the microparticle is coated with streptavidin. In some instances, the microparticle coated in biotin is bound to streptavidin. In some instances, the microparticle is porous agarose beads. In some instances, the microparticle is an agarose bead. In some instances, the agarose bead is about 70 to about 100 μm in diameter. In some instances, the quantum dot is immobilized on the microbead through a non-covalent bond, antibody-antigen conjugation, inorganic ligand coatings, covalent linkages as achieved through click chemistry, or other nucleic acid, protein or biomolecular surface conjugates.

According to various aspects of the disclosure, the quantum dots are conjugated to antibodies. In some instances, the antibodies conjugate to proteins in the extracellular matrix, such as, for example, collagen and fibronectin. In some instances, the antibodies conjugate to proteins for targeting quantum dots to specific body parts, for example: hyaluronic acid, cartilage oligomeric matrix protein, collagen type II, citrullinated proteins, elastin, keratins, proteoglycans, tenascin-C, osteocalcin, alkaline phosphatase, and osteoblast.

In accordance with various aspect of the disclosure, after injection with quantum dots, the animal is placed in a staging area to measure fluorescent intensity of the quantum dot. In some instances, the fluorescent intensity is measurable from about 1 day to about 120 days post injection, alternatively about 50 to about 120 days post injection, and alternatively about 100 to about 120 days post injection.

In accordance with various aspects of the disclosure the fluorescence of the quantum dots is captured or recorded. In some instances, the fluorescence of the quantum dots is captured from multiple angles. In some instances, the animal is surrounded by devices that can capture the fluorescence emitting from the animal. The multiple angles allow an accurate 3D reconstruction of fluorescent key points under the animal's skin as they move around. In some instances, the cameras capture at least 10 frames of the moving fluorescent animal. In some instances, the cameras capture at least 100 frames of the moving fluorescent animal. In some instances, the cameras capture at least 1000 frames of the moving fluorescent animal. In some instances, the cameras capture at least 10000 frames of the moving fluorescent animal. In some instances, the cameras capture at least 100000 frames of the moving fluorescent animal. In some instances, the cameras capture at least 1000000 frames of the moving fluorescent animal. In some instances, the cameras capture at least 10000000 frames of the moving fluorescent animal. In some instances, the fluorescence can be tracked by using computer software. In some instances, the tracking computer software is SLEAP. In some instances, the tracking computer software is DeepCutLab. In some instances, the tracking computer software is another deep neural network. In some instances, the fluorescence of the quantum dots is captured through camera. In some instances, the camera is a near-infrared camera. In some instances, the excitation and emission optics on the camera is about 650 nm to about 1000 nm. In some instances, the frames are used to track the movements of joints within an animal. In some instances, the system can resolve three-dimensional pose with sub-mm precision, which is required to accurately reconstruct movements such as locomotion, rearing, and grooming in small laboratory animals, for example, mice. In some instances, specific behaviors are identified using state-space models, for example Motion Sequencing (MoSeq). The frequency of behaviors, and the transitions between them, can be used to quantify whether a mouse is healthy.

In accordance with various aspects of the present disclosure, the animal is placed in a recording arena. In some instances, the recording arena is made of plexiglass. In certain aspects of the present invention, the animal is injected with a quantum dot, and then has their fluorescence measured for one or more days.

In accordance with various aspects of the present disclosure, the animal to be injected in a diseased animal. In some instances, the disease is cancer. In some instances, the cancer is a blood cancer. In some instances, the cancer is a solid tumor. In some instances, the disease is a neurodegenerative disorder. In some instances, the neurodegenerative disorder is Alzheimer's, dementia, Ataxia, Huntington's disease, Parkinson's disease, motor neuron disease, multiple system atrophy, or progressive supranuclear palsy. In some embodiments of the present invention, movement loss in neurodegenerative diseases is measured through injection of the quantum dots in the animal. In some instances, the animal is an animal that has been treated (e.g. with a drug). In some instances, the animal is a genetically-modified animal. In some instances, the genetically modified animal is a transgenic animal. In some instances, a gene is knocked out in the animal. In some instances, a gene is inserted into the animal. In some instances, the transgenic animal is a conditional model.

In accordance with various aspects of the present disclosure, the animal has their movement captured via measuring the fluorescent particles. In some instance the movement is captured to look for differences between diseased animals and healthy animals. In some instances, the gait is measured in the animals. In some instances, the animal in measured for a halting gait. In some instances, the animal is measured for a tremor. In some instances, the animal is measured for dyskinesia. In some instances, the dyskinesia is due to L-DOPA treatment. In some instances, the animal is measured for tics. In some instances, the animal is measured for chorea. In some instances, the movements measure, such as gait or tics, is compared between a diseased animal and a healthy animal. In some instances, all behaviors can be resolved using unsupervised machine learning, for instance state space models such as MoSeq.

In accordance with various aspects of the present disclosure, the quantum dots are designed for different conditions. In some instances, the quantum dots are designed for tracking the vasculature. In some instances, the quantum dots are designed for labelling cells. In some instances, the quantum dots are designed where biotinylated quantum dots are attached to porous streptavidin-coated agarose beads.

### Examples

Fluorescent nanoparticles called “quantum dots” are utilized to visualize key points in vivo. Quantum dots are semiconductor nanoparticles that are highly photostable, have minimal photobleaching, and high quantum yield at wavelengths ranging from blue to near-infrared (NIR) (Reineck & Gibson, 2017). To noninvasively image through the fur and soft tissues, near-infrared (NIR) quantum dots may be used to image wavelengths within the NIR-I spectrum. Quantum dots are introduced into the body through injecting with a customized Drummond (Drummond Scientific, Cat. No. 3-000-510) and glass pipette.

First, it was established that quantum dots can be imaged through the skin by injecting quantum dots subdermally in the subcutaneous fatty tissue. Second, using subdermal injections, the inventors identify specific quantum dot formulations that serve as bright, long-lived physical fiducial markers. That includes antibody-conjugated quantum dots that can be targeted to ultra-long-lived proteins like collagen to further enhance signal longevity in vivo up to 4 months post-injection. This phase of testing also presented an opportunity to collect a large dataset for reference point markerless keypoint trackers, wherein subdermal quantum dots and body surface reflectance were imaged simultaneously. This dataset is to benchmark the performance of an existing markerless keypoint tracker, SLEAP (10), relative to quantum dot fiducials embedded in the skin. It was found that quantum dots can be used to enhance the accuracy of markerless keypoint trackers. Finally, quantum dots can be injected intra-articularly for directly tracking joint kinematics. The joints impose constraints on skeletal movement, and imaging joint motion in 3D is an important step towards the overarching goal of directly measuring skeletal kinematics. Ultimately, QD-Pi addresses a critical need in the study of motor control in neuroscience and sets the stage for a generalizable method to track movement and skeletal kinematics in freely moving animals.

To immobilize quantum dots on microbeads, a streptavidin-biotin linkage was utilized which creates a strong non-covalent bond, although other covalent or noncovalent conjugation approaches may deliver a similar effect (some examples include but are not limited to: biotin combined with any other binding proteins such as avidin or NeutrAvidin; antibody-antigen conjugates; inorganic ligand coatings; covalent linkages as achieved through Click chemistry; or other nucleic acid, protein or biomolecular surface conjugates). To maximize spot brightness, high-capacity microbeads were utilized and composed of biocompatible agarose that can be saturated with quantum dots throughout their 3D structure due to their porous nature. Specifically, experiments combined biotinylated quantum dots with streptavidin coated high-capacity agarose beads (Amid Biosciences, #SA-101-1); but the reverse combination may also be equivalent (e.g., streptavidin conjugated quantum dots combined with biotinylated high-capacity microbeads); and other biocompatible high-capacity beads composed of porous substances other than agarose may also achieve the desired effect. Quantum dots and beads are combined, incubated, washed via centrifugation, and finally resuspended in a density-matched sodium alginate solution to allow for an even distribution of beads during injection. The suspension is injected into the animal and imaged. Bright fluorescence was observed that lasts for multiple weeks. Histology revealed that the beads form stable deposits in the skin, which contributes to the observed longevity. The described new protocol is an attractive alternative to the previously established one as it can be used for neuroscience experiments that require longer timescales.

### Quantum Dots are Viable as Injectable Optical Tags in Mice

A protocol was developed to inject quantum dots subdermally into mice to establish that quantum dots can be imaged through the skin while mice freely move in an arena. Quantum dots were used as a fluorescent marker because of their brightness, photostability, and broad emission spectral profiles within the NIR-I (650-900 nm) range, which is an ideal window for imaging through skin (33-35) and blood (36). Quantum dots are also biocompatible, and their surface chemistry can be readily modified with a variety of biologically functional surface coatings. Here, quantum dots were utilized with a fluorescence emission peak at 800 nm (referred to as QD800 throughout) coated in polyethylene glycol (PEG), previously demonstrated to be biocompatible when introduced into animals (45)).

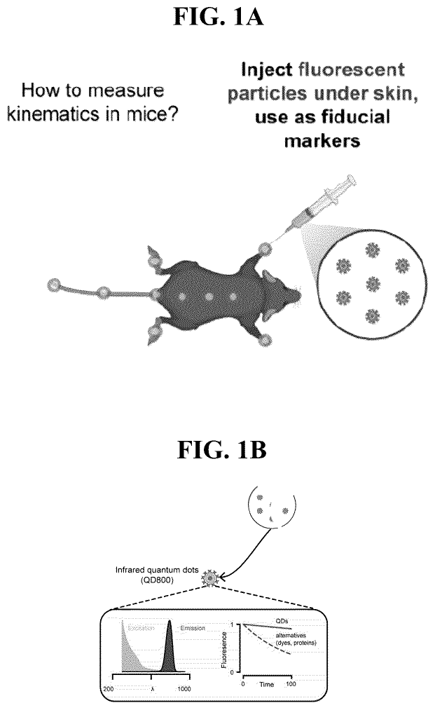

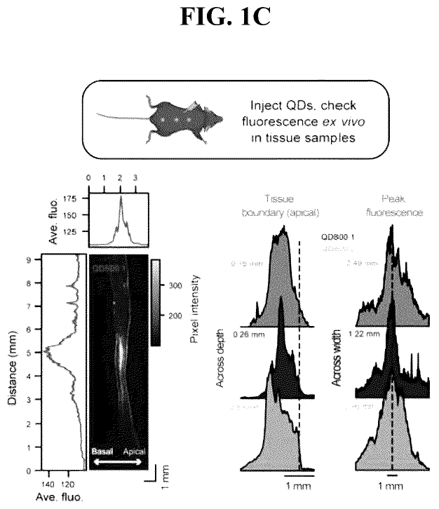

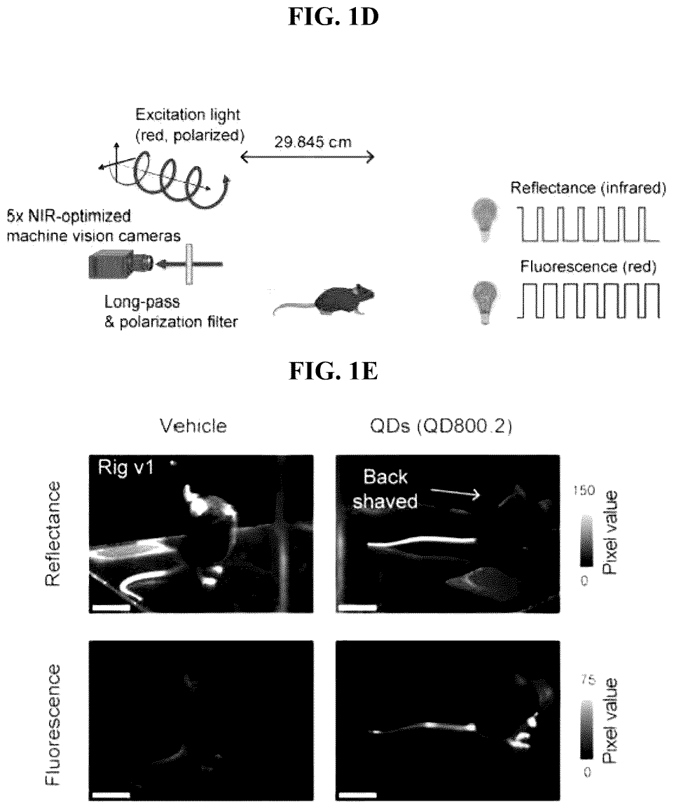

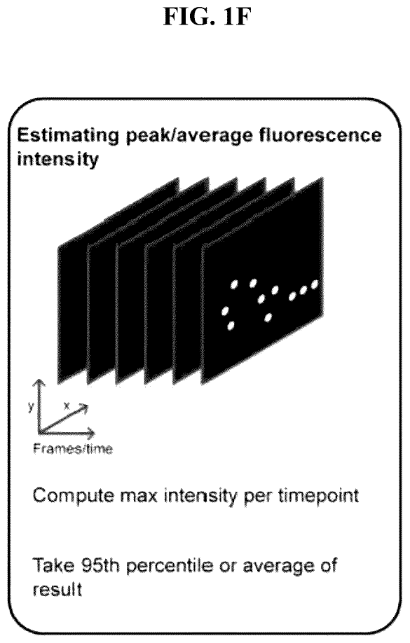

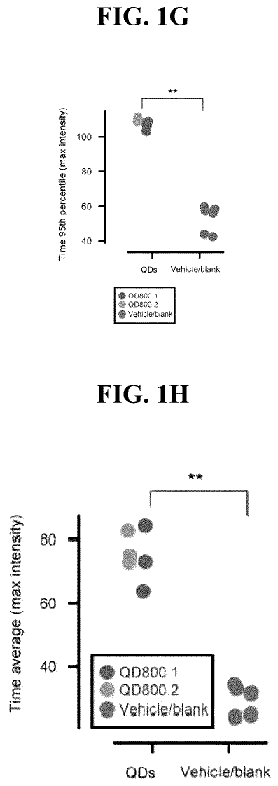

Quantum dots were first tested to determine if they could form discrete “tags” without dispersing through the skin and underlying tissue (FIGS. 1A, 1B, and 1C). Ideally, the resulting tag would be large enough to resolve on a standard machine vision camera, yet small enough to mark many different points on the body. As an approximation, it was assumed as a basic criterion that fluorescence points should be at least 1 mm in diameter to enable high-resolution tracking (roughly 5 pixels assuming an object distance of 12 inches to the camera based on the camera calibrations).

While Qdot 800 particles are an ideal candidate for acting as an optical tag, their ability to form aggregates with appropriate properties for imaging after injection into the skin is not well established. Therefore, the first step was to characterize their ability to form spatially contiguous “tags” without dispersing in the skin. Additionally, if they formed a tag, it was necessary to know that the tag would have an appropriate spatial scale for non-invasive imaging; that is, the tag would need to be large enough to resolve on a standard machine vision camera, yet small enough to mark different points on the body. As an approximate rule of thumb, it is assumed that this means fluorescence points should be at least 1 mm in diameter (roughly 5 pixels based on the camera calibrations) and no more than 10 mm in diameter—the distance between many key landmarks on the mouse (Ramalingasetty et al., 2021).

To address these questions, a simple micro-injection protocol was used whereby small quantities (<5 μL) of Qdot 800 particles could be injected into the skin of a briefly anesthetized live mouse (see Methods). Here, it was speculated that Qdot 800 vascular labels, a formulation originally designed for injection into the tail vein, would be an attractive first candidate due to their previous use in vivo (Qdot 800 variant 1, see Methods). In order to visualize the spatial properties of the injected particles, they were injected into the skin layer of the back and then harvested and the tissue sectioned for visualization under an epifluorescence microscope (FIG. 1C).

Histology revealed that Qdot 800 variant 1, when injected into the skin, formed an aggregate that was on average 1.9 mm (±0.2 mm SD) wide and 0.72 mm (±0.12 mm SD) deep. It was speculated that these length scales were appropriate for non-invasive imaging using machine vision cameras equipped with standard lenses (camera calibration determined that 2 mm spanned approximately 10 pixels).

To accomplish this, a simple micro-injection protocol was developed whereby small quantities (<5 μL) of QD800 particles could be injected under the skin of a briefly anesthetized live mouse using a glass micropipette (see Methods). Two QD800 formulations were selected. Vascular labels, a quantum dot suspension originally designed for visualization of the vasculature (referred to here as QD800.1), and cell labels, a quantum dot suspension of nanoparticles functionalized with a peptide that facilitates its uptake into cells (referred to here as QD800.2) for use in initial tests based on their prior use in vivo and desirable spectral properties. Three subdermal locations were identified along the back (dorsal midline) for injection and confirmed the size and depth of the fluorescence spots in extracted ex vivo tissue sections (FIG. 1C). Histology revealed that injections of QD800.1 or QD800. 2 yielded a bright fluorescent spot in adipose tissue just beneath the dermis, at a depth of 0.26-0.83 mm beneath the skin with a spot full-width at half maximum of 1.22-2.96 mm (approximately 6-15 pixels in images acquired with machine vision cameras equipped with standard 8 mm focal length lenses at a distance of 12 inches, the approximate distance from each camera to the center of the arena in the setup). This then extended the method to 14 subdermal locations: dorsal and ventral injections to each paw, three injections along the tail, and three injections along the spine (FIG. 1A). It is important to note that injection areas that involved fur required shaving and treating with hair removal cream so that fur did not interfere with imaging.

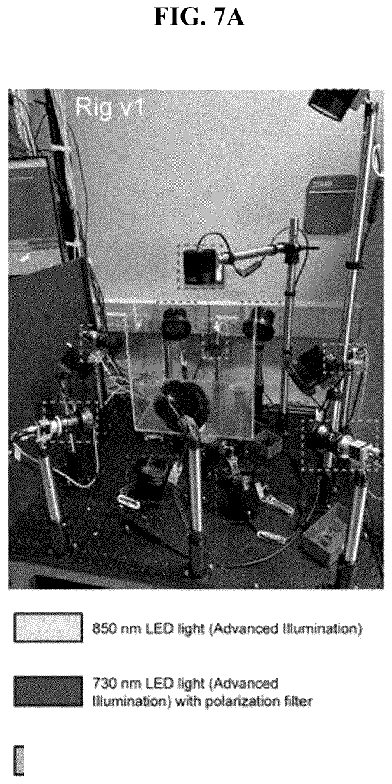

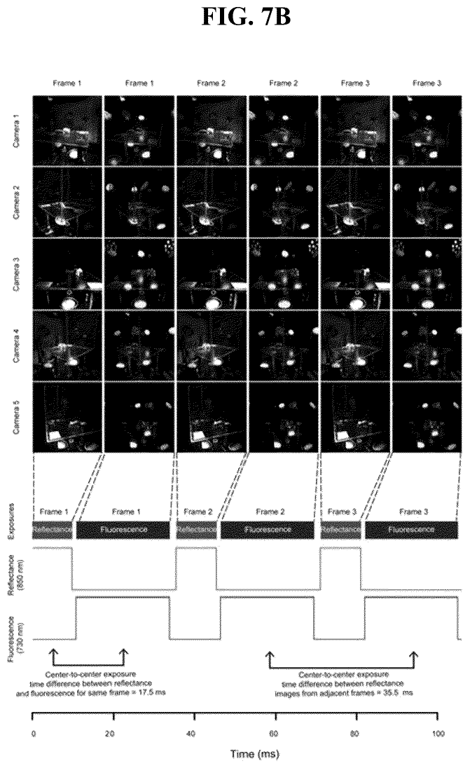

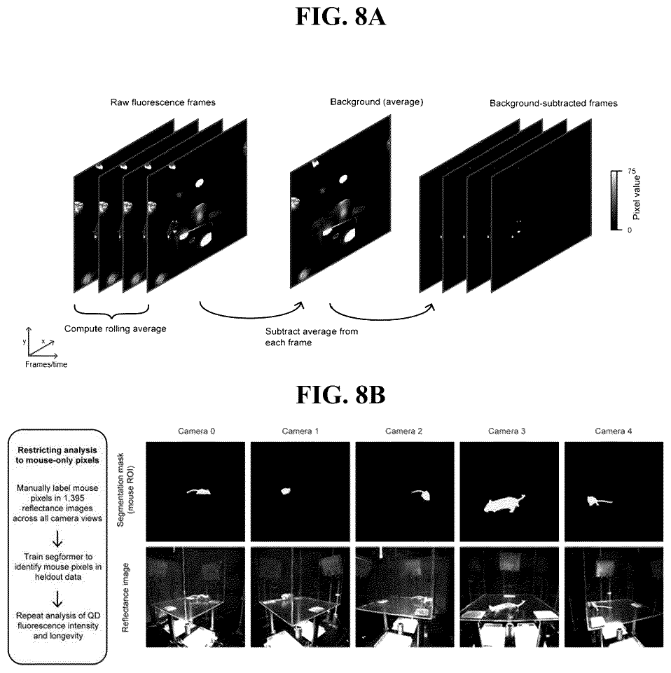

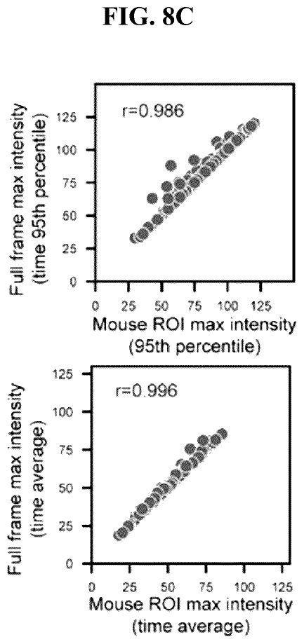

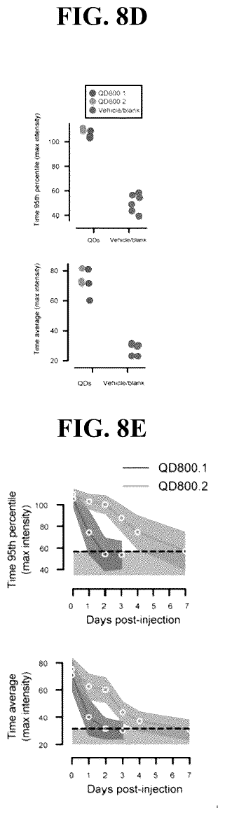

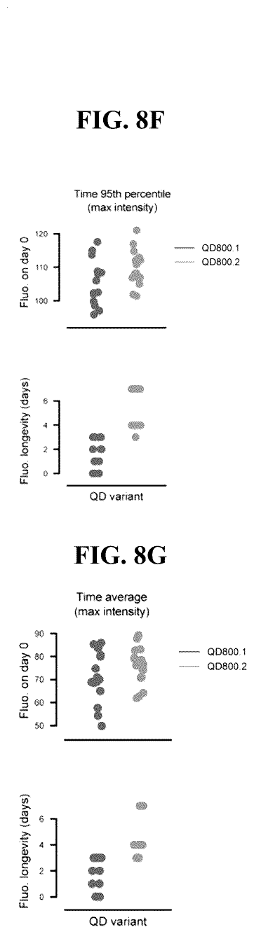

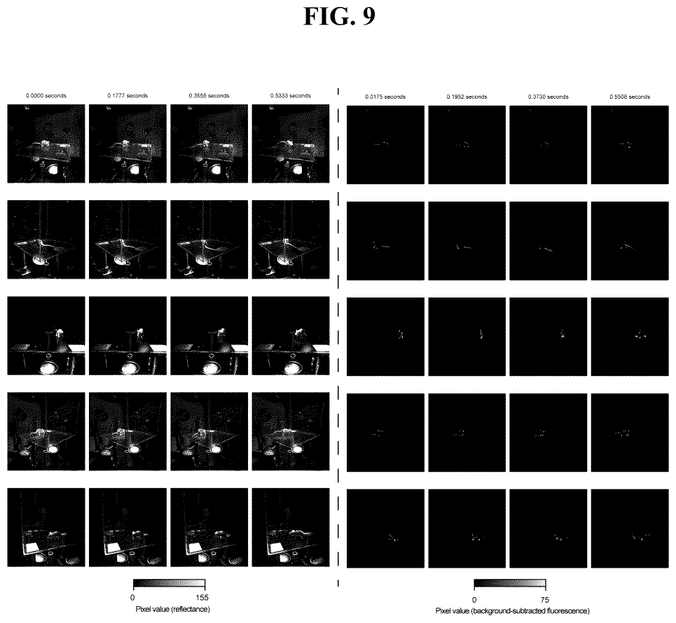

In order to image QD800.1 or QD800.2 in freely moving mice, a plexiglass imaging arena was constructed surrounded by five NIR-optimized machine vision cameras, which was an open-top cube with an edge length of 29.845 cm (FIG. 1D, FIG. 7). The cameras were equipped to collect both reflectance images to visualize the location of the mouse and fluorescence images to visualize QD800 fluorescence (730 nm excitation, >800 nm emission; this lighting setup is referred as Rig v1 as seen in FIG. 7). To explicitly estimate the contribution of non-QD dependent signals in the fluorescence channel, a separate group of animals received either vehicle or no injection (see Methods). Following optical tag embedding, mice were introduced into the plexiglass arena and were imaged during free behavior over three to five-minute-long sessions (FIG. 1E). Relative to vehicle-injected mice, mice injected with QD800.1 or QD800.2 exhibited clearly resolvable fluorescence (signal-to-noise ratio ˜2.51±0.25 SD, where it was defined signal-to-noise ratio as average fluorescence across the whole image per QD-injected mouse relative to average blank/vehicle-injected mice, n-6 QD800 injected mice, composed of n=3 QD800.1 and n=3 QD800.2 mice, and n=6 blank/vehicle-injected mice, (FIGS. 1F, 1G, and 1H, FIG. 8).

### As Free Nanoparticles, Injected Quantum Dot Signal Decays on the Timescale of Hours In Vivo

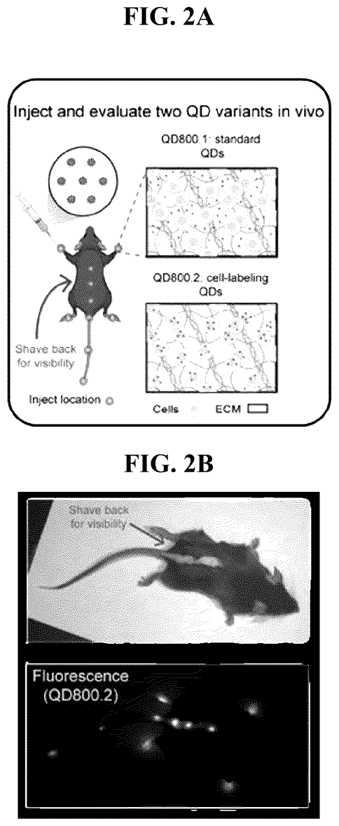

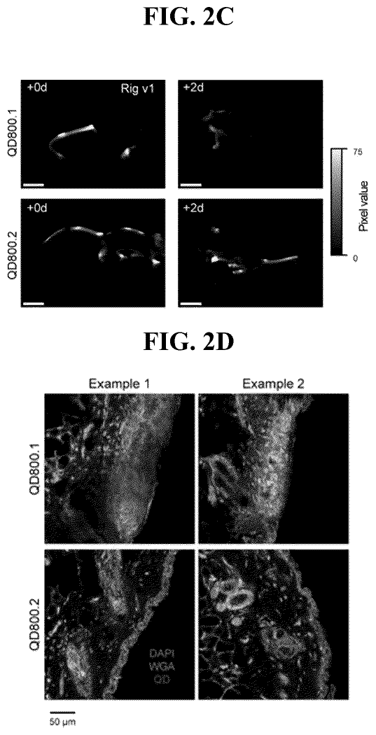

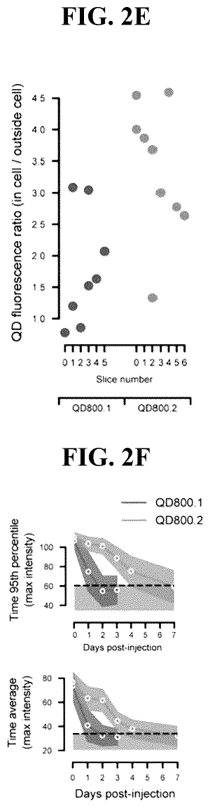

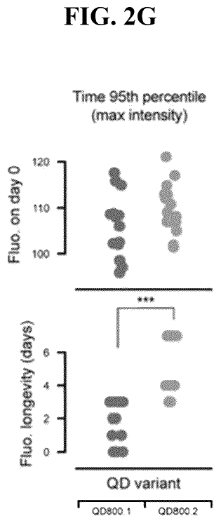

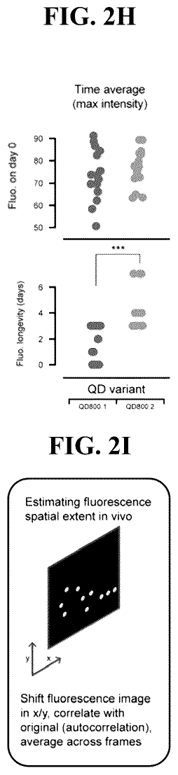

For future applications of the method in standard neuroscience experiments (e.g., photometry, imaging, pharmacological or optogenetic manipulations) or for longitudinal tracking of an animal's movements, the optical tags should retain fluorescence for as long as possible—ideally on the order of weeks. To test the longevity of QD800 fluorescence, fluorescence was monitored in freely moving mice across multiple days after subdermal injection (FIGS. 2A, 2B, and 2C). As QD800.1 is a suspension of nanoparticles (10-20 nm diameter) without targeting tags, it was speculated that they would quickly disperse between cells within a tissue. On the other hand, QD800.2 is a suspension of nanoparticles functionalized with a peptide that facilitates its uptake into cells (FIGS. 2A and 2B), which was speculated would lead to enhanced fluorescence longevity in vivo.

Longitudinal imaging of mice injected with Qdot 800 variant 1 and 2 (n=3 mice each, see Methods) revealed that variant 1's fluorescence decayed within 1.6 (±1.4 SD) days, while variant 2's fluorescence decayed within 5.3 (±1.7 SD) days (FIGS. 2D, 2E and 2F, see FIGS. 8 and 9). That confirmed the speculation that cell internalization could enhance fluorescence longevity. Additionally, it was desired to characterize the spatial profile of the injections in vivo to confirm the extent of the fluorescence points in each frame. Therefore, the average spatial autocorrelation of the fluorescence images was calculated across all animals and camera views and it was found that Qdot 800 particles exhibit structured distribution in vivo for both variants (FIGS. 2G and 2H).

Attaching Odot 800 to Micro-Scale Particles Leads to Fluorescence that Persists for Weeks.

To visualize the dynamics of these quantum dots in the skin, QD800.1 and QD800.2 injections were performed along the back of an additional cohort of animals and harvested the tissue for histology (n=4 tissue samples/injections, n=2 with QD800.1 and n=2 with QD800.2, see Methods). The cell membranes and nuclei were stained and the fluorescence was visualized with confocal microscopy in multiple fields of view to confirm whether the quantum dots were located inside or outside of the cells (FIG. 2D). It was then quantified the degree to which quantum dots entered the cells as the ratio of the average quantum dot fluorescence inside compared to outside of cells. It was found that the median ratio (intracellular/extracellular) for QD800.2 (3.7) was more than two-fold greater than the ratio for QD800.1 (1.6) (FIG. 2E).

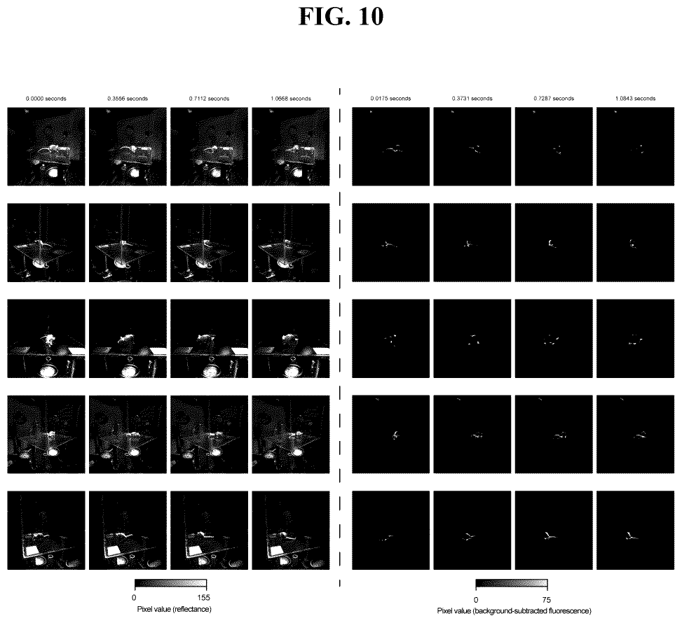

Longitudinal in vivo imaging of mice injected with QD800.1 and QD800.2 revealed that signal decayed within 1.6±1.4 days and 5.3±1.7 days, respectively (mean±SD; FIGS. 2F, 2G, and 2H, see FIGS. 9-10 for raw data). That supported the hypothesis that cell internalization could prolong visualization of the optical tag. Since QD800.1 and QD800.2 both use the same underlying fluorescent nanoparticle, it also suggested that the decay in fluorescence was not due to photobleaching. Average spatial autocorrelation of the fluorescence images across all animals and camera views showed that both QD800 variants remained localized to the injection site in vivo immediately following injection (FIGS. 21 and 2J).

Even though Qdot 800 variant 2 significantly outlasted variant 1 in terms of fluorescence, it was speculated that attaching Qdot 800 to a particle larger than cells would increase their longevity even further as it would prevent the nanoparticles from migrating. Prior work has shown that polymethyl-methacrylate (PMMA) beads can be introduced into human skin and can last for multiple years (Lemperle et al., 1995). Thus, it is hypothesized that attaching quantum dots to a microparticle with a similar length scale as PMMA beads (30-40 μm) could yield improved longevity.

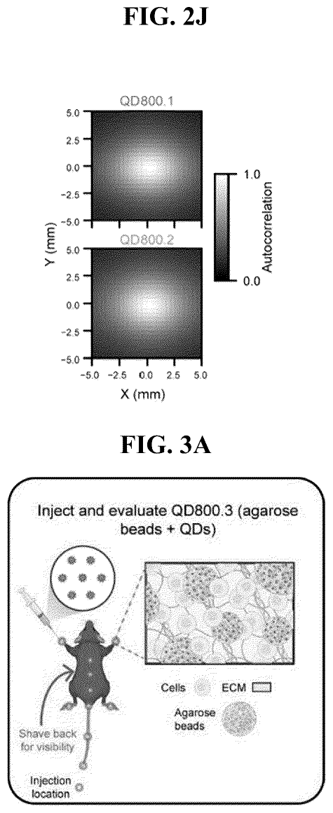

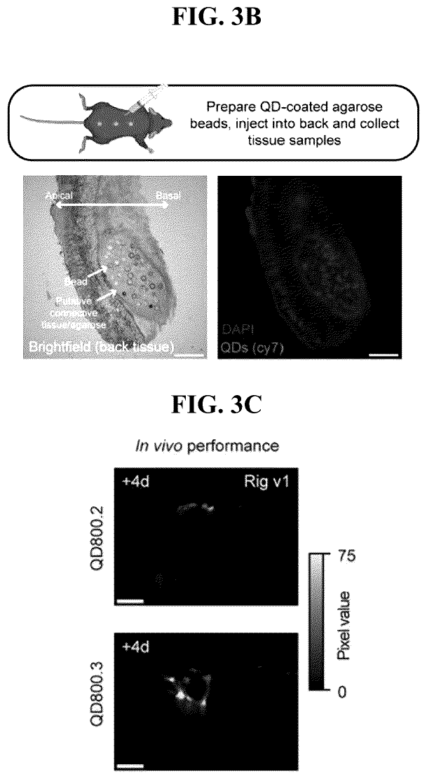

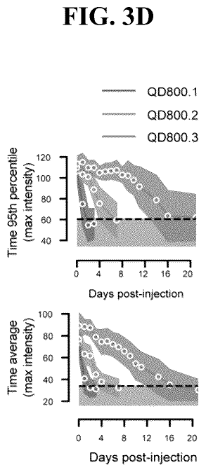

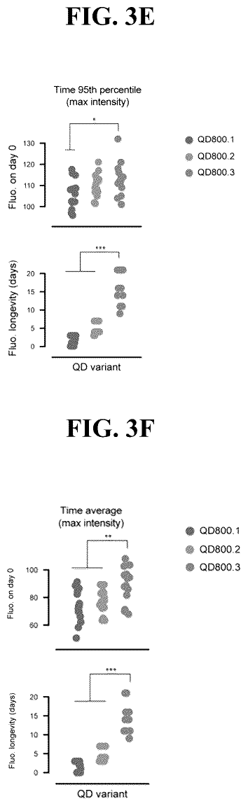

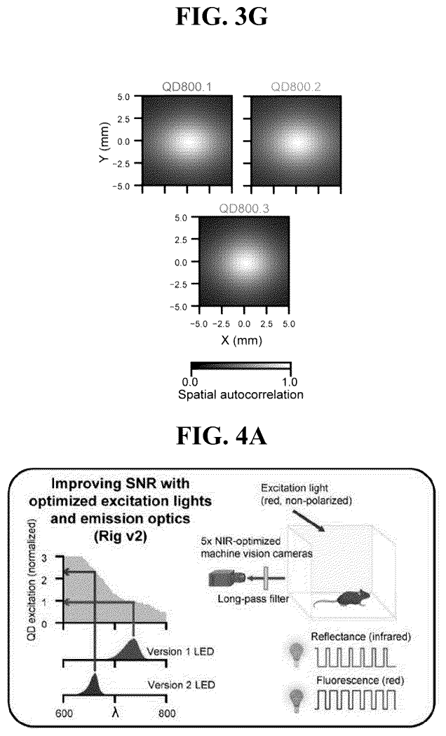

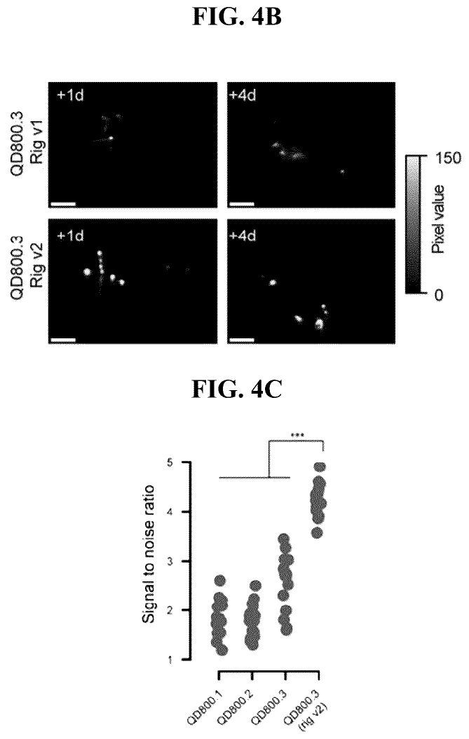

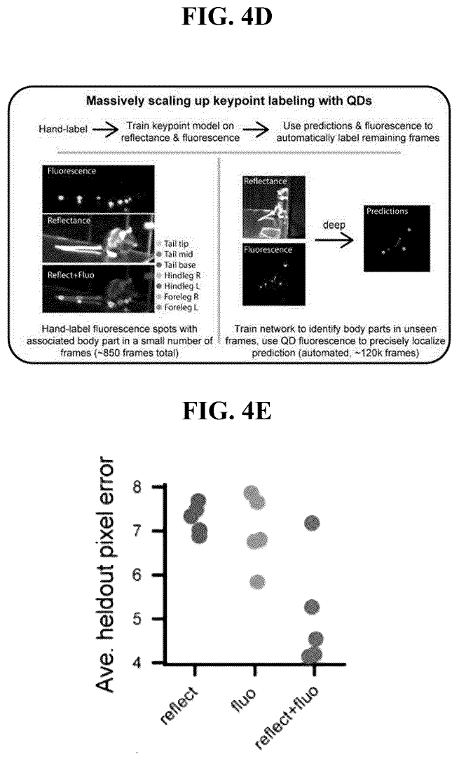

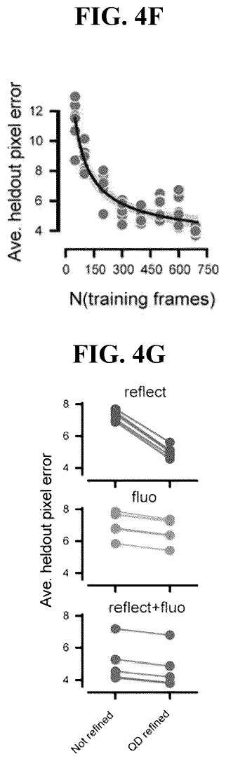

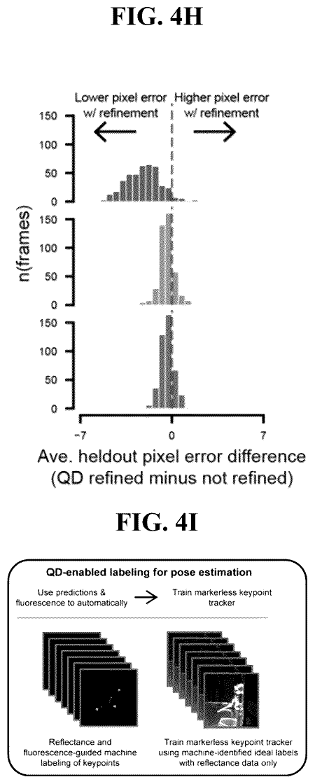

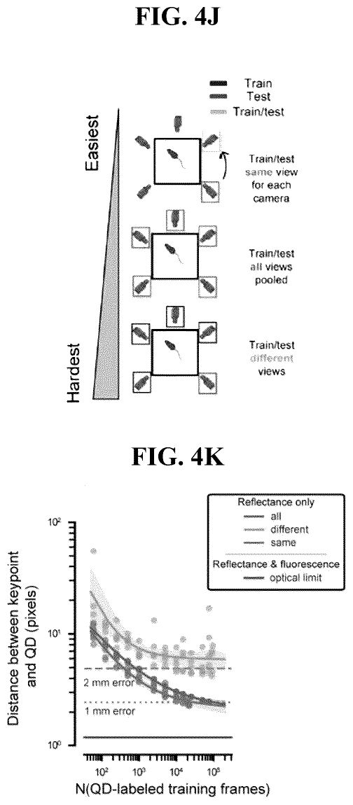

Here, the next step turned to the streptavidin-biotin interaction—one of the strongest non-covalent bonds found in nature. Microscale streptavidin beads are widely available due to their widespread use in purification assays, and quantum dots can be purchased with biotin coatings. However, in the current experiment's case, the beads needed to be: (1) approximately size-matched to beads that have previously been shown to form stable implants (10s of μm), (2) small enough to be injectable using the micro-injection system, (3) biocompatible, (4) have the capacity for high-quantum dot loading. One of the few types of streptavidin beads to satisfy all four requirements is high-capacity agarose beads, which have a porous structure that allows quantum dots to attach to a large surface area (see Methods). Hence, a protocol was established for high-capacity agarose beads with biotinylated quantum dots—Qdot 800 variant 3 (FIGS. 3A, 3B, 3C, 3D, and 3E). It was found that Qdot 800 variant 3 had extremely bright fluorescence, had a diameter roughly matched to prior work with PMMA beads (85.4 μm diameter±12 μm SD), and formed dense aggregates in the skin layer (FIGS. 3F and 3G, FIG. 10). And most importantly, loading Qdot 800 onto beads radically improved their longevity in vivo—from on average 5.3 days in variant 2 to 16.5 (±4.7 SD) days in variant 3 (n=3 mice, FIG. 4C; see Methods)—without any significant decline in overall brightness or spatial scale (FIG. 4F).

Microbead-Immobilized Quantum Dots Yield a Bright Fluorescent Spot that can be Imaged for Weeks

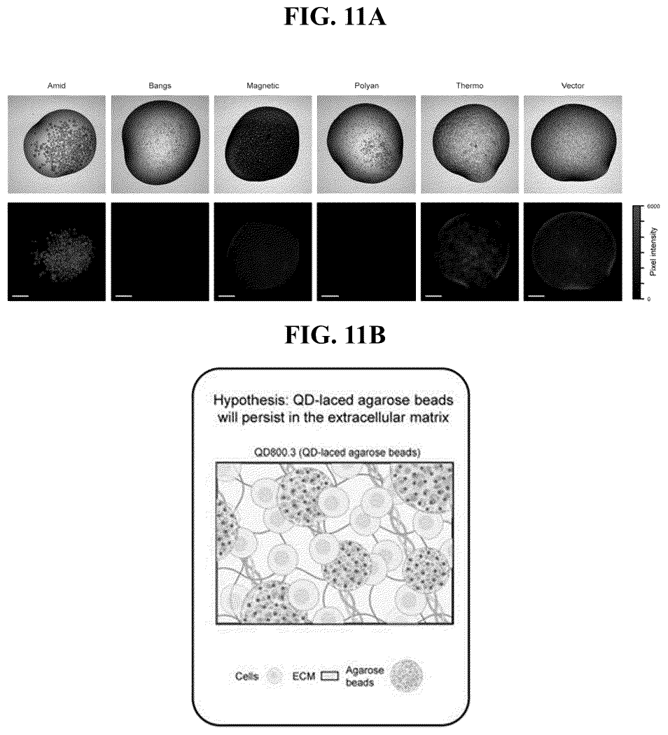

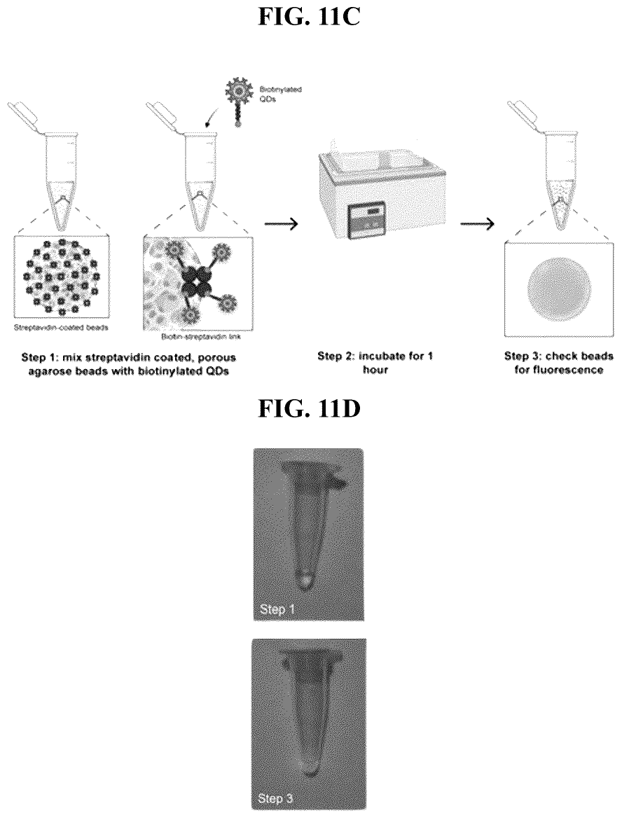

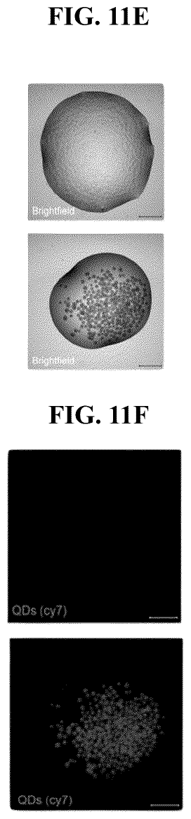

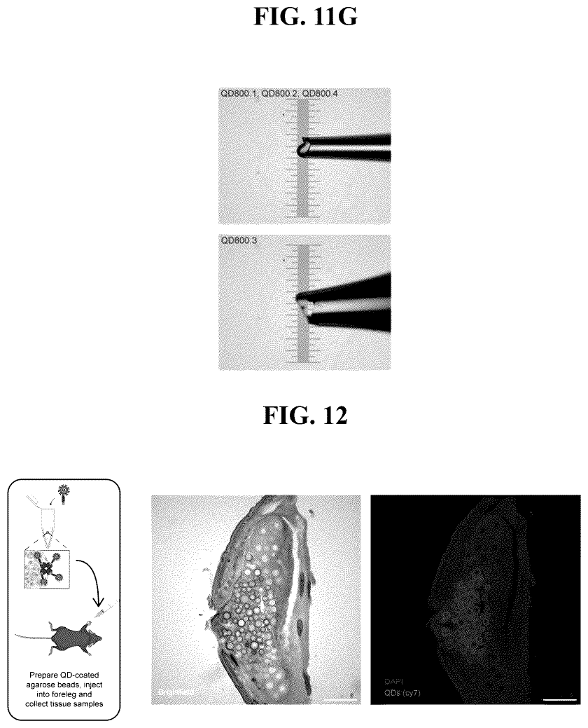

Even though QD800.2 outlasted QD800.1, each was only detectable for days, not weeks. It was hypothesized that immobilizing quantum dots on large microparticles would further prolong the signal of the optical tag by minimizing diffusion and discouraging clearance from the body. Prior work has shown that >30 μm diameter microbeads injected subdermally in humans can form long-lasting deposits that persist for multiple years (46). It was therefore tested by combining biotin-functionalized quantum dots with streptavidin-functionalized microbeads (QD800.3; FIGS. 11A, 11B, 11C, 11D, 11E, and 11F) and injecting them subdermally as before. Six commercially available microbeads were tested and found that porous approximately 85 μm diameter high-capacity agarose beads yielded bright beads (FIG. 11A). It was confirmed that QD800.3 formed dense deposits in the skin that persisted in the animal (3A, 3B, and 3G, FIG. 12), increasing the imaging window to >16 days (16.6±4.6 days; QD800.3, n=3 mice, FIGS. 3C, 3D, and 3F). That longevity is critical to tracking the progression of movement disorders in certain mouse disease models, such as models for Parkinson's disease (47), and over the course of standard systems neuroscience experiments that include reading and writing neural activity over multiple sessions

### Optimizing Excitation Wavelength and Imaging Optics Maximizes Signal-to-Noise

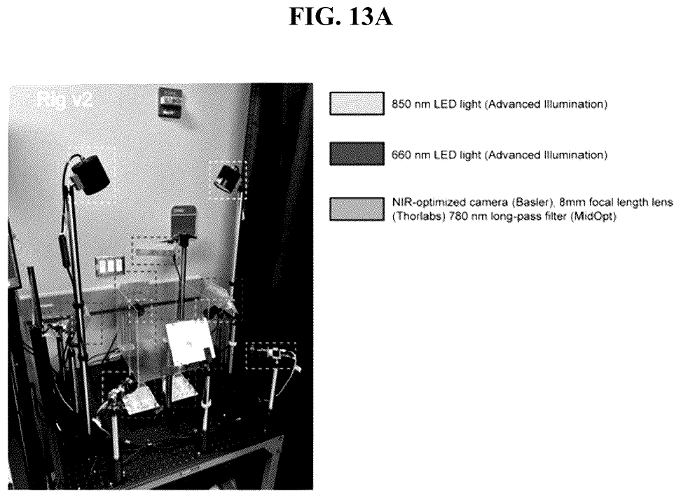

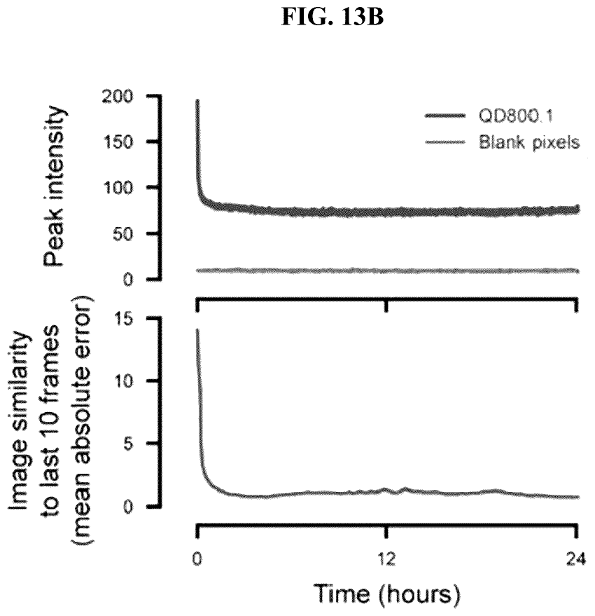

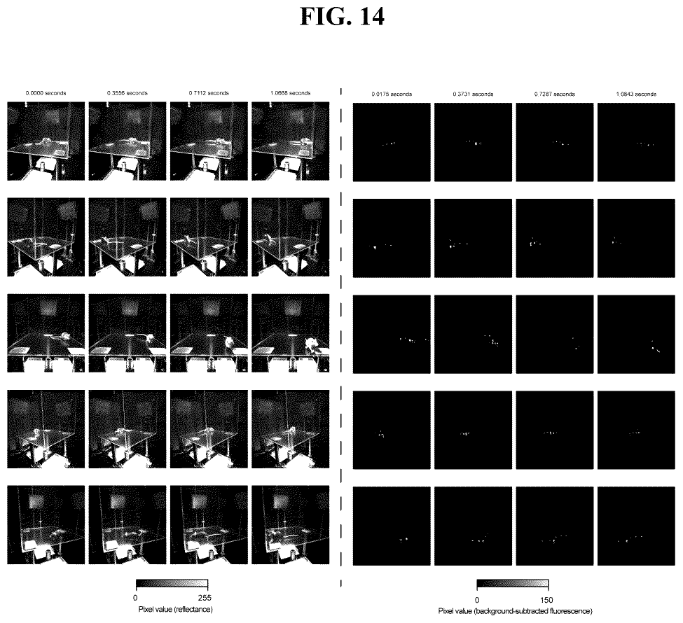

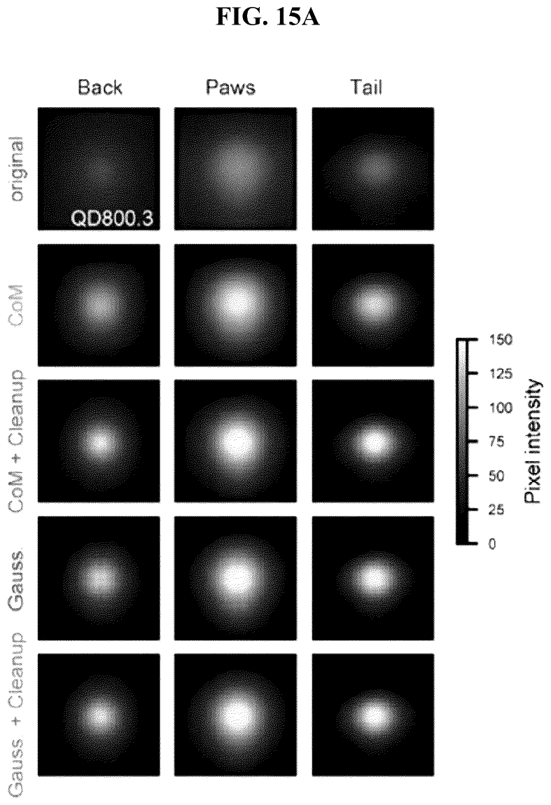

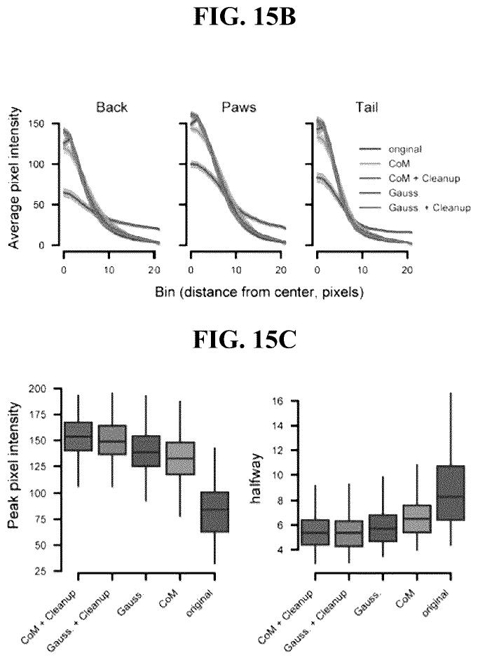

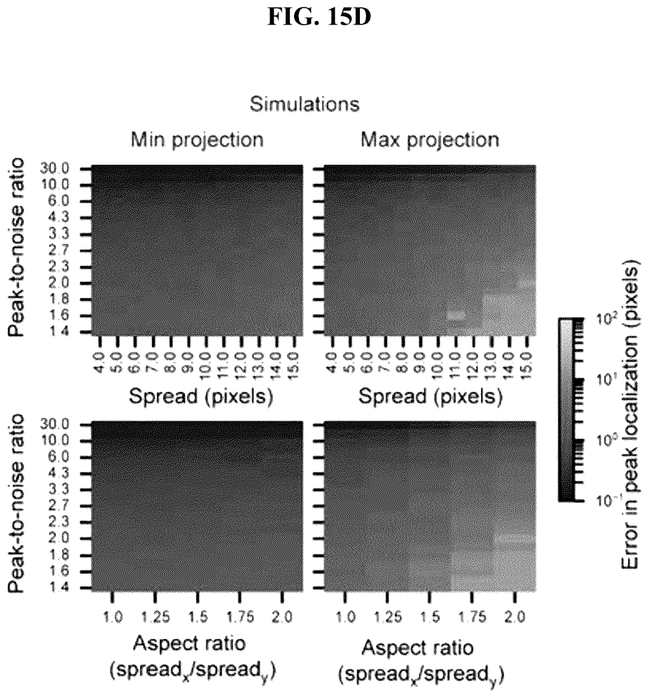

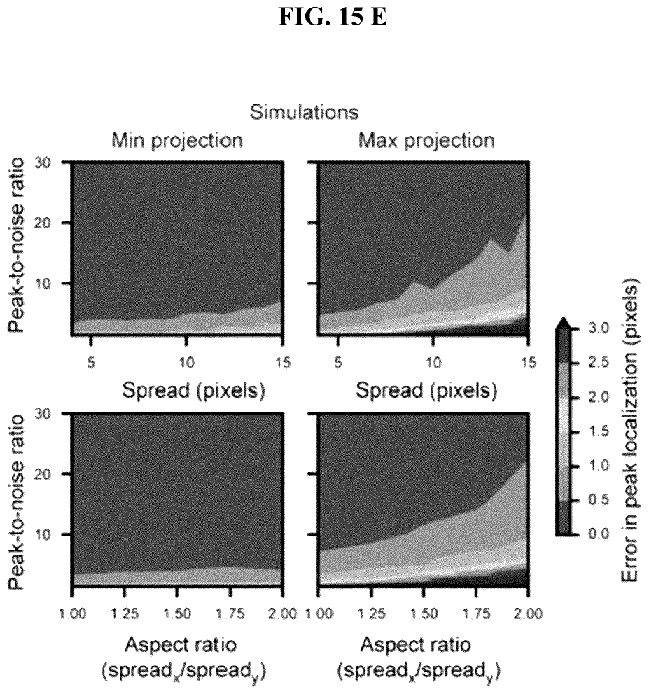

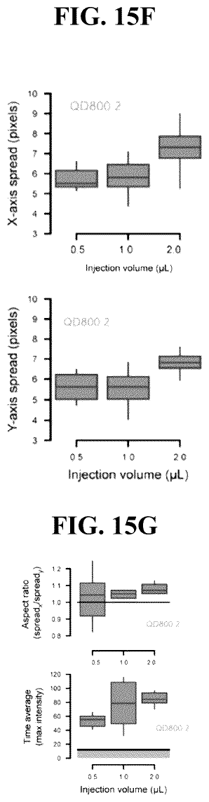

Having confirmed the use of quantum dots as discrete, bright, long-lived optical tags, it was next hypothesized that the signal-to-noise ratio (SNR) could be improved by optimizing the imaging hardware (camera, excitation lights, and optical filters). Specifically, it was speculated that tuning the excitation and emission optics to better match the optical properties of QD800 without being appreciably visible to the mouse (wavelengths equal to or above 650 nm), could substantially improve fluorescence signal (+8). Initial recordings were made using 730 nm LEDs with polarization filters for excitation, and cameras were equipped with 830 nm longpass filters and polarization filters tuned to the orthogonal orientation to minimize stray light (Rig v1 as seen in FIG. 7; FIG. 4A). While 730 nm is ideal for penetrating the skin, it only excites QD800 particles at 1% efficiency, and an 830 nm longpass filter clips the peak emission (35). Hence, the setup was altered to use 660 nm LEDs, thereby doubling the excitation efficiency, and a 780 nm longpass filter to maximize collection of the emitted light (Rig v2; FIG. 4A, FIG. 13A). These changes increased SNR>2-fold for mice injected with quantum dots subdermally as before (FIGS. 4B and 4C, FIG. 14), which provided unambiguous detection of keypoints with noise characteristics suitable for use in downstream applications (FIG. 15), such as training markerless keypoint models. This final iteration of QD800.3 tags with the upgraded imaging system and analysis pipeline combined to form QD-Pi.

### Imaging Datasets of OD800-Tagged Mice can be Used to Profile and Train Markerless Keypoint Tracking Algorithms to Reach New Levels of Precision and Robustness

Having built QD-Pi, it was set out to collect a unique, large-scale dataset that could be used to both train and benchmark markerless keypoint trackers. Specifically, rapidly alternating reflectance and fluorescence images were collected from freely moving mice injected with QD800.3 subdermally (n=3 additional mice), which enabled the collection of high-quality video-rate data surface features of the mouse registered to ground truth key points inside of the mouse (FIG. 4D). This system allowed the ability to test the accuracy of commonly used markerless keypoint tracking algorithms relative to reference point optical markers placed inside of the body. Here, experiments were focused on the commonly used U-Net architecture available in the SLEAP package (10).

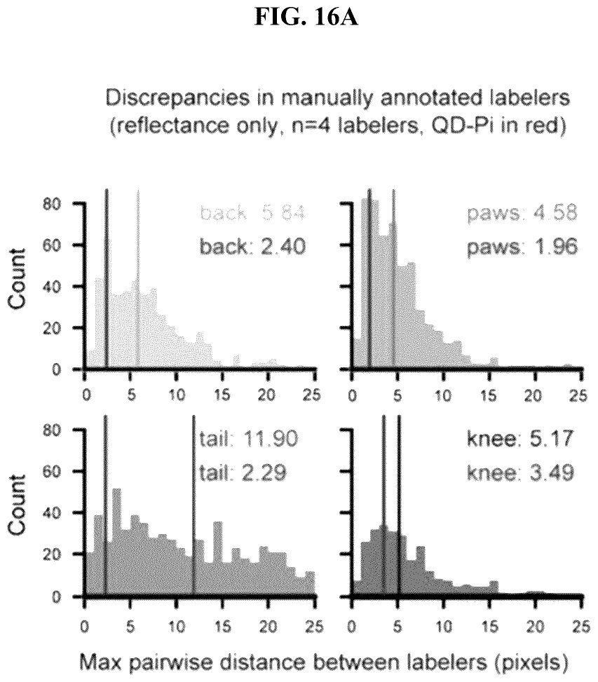

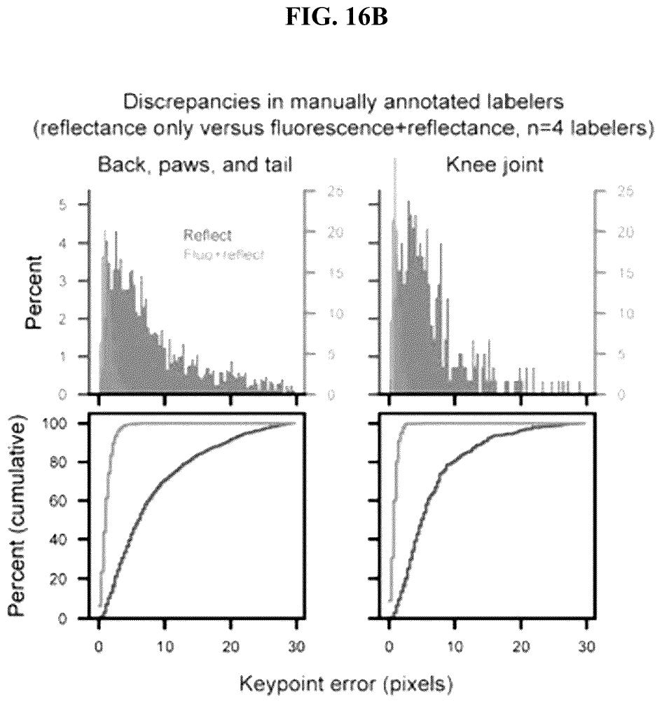

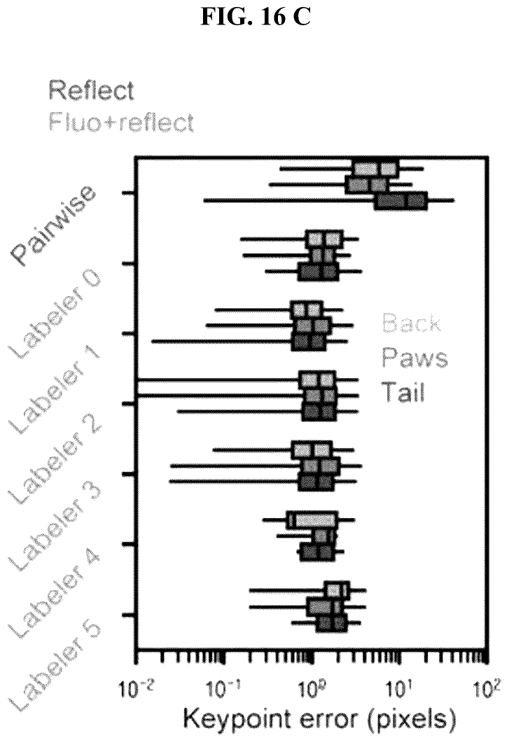

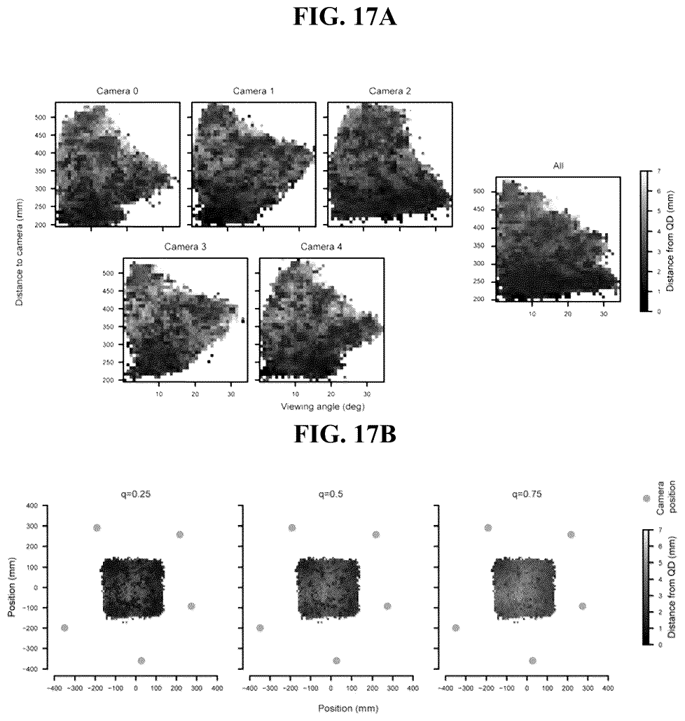

The first goal was to automatically label the body part associated with each QD800.3 injection. To accomplish that, 862 frames were hand-labeled across the five camera views (see Methods). Fluorescence and reflectance frames were superimposed so that the labeler could identify both a given body part and peak QD800 fluorescence (FIG. 4D). Those hand-labels were then used to train a U-Net using both reflectance and fluorescence data, to identify which quantum dots correspond to which body part (FIG. 16). The resulting dataset comprised 114,629 frames of machine-labeled keypoints in three mice across five camera views, which was called QD-Pi-120K (see Methods). That presented an opportunity to test the scaling properties of common markerless keypoint trackers relative to reference point fiducial markers (FIGS. 4E, 4F, 4G, and 4H). It was speculated that such a large dataset would enable us to train individual networks that could identify keypoints across all five camera views, and networks that could generalize to novel views-a particularly challenging problem given the small size of hand-labeled datasets typical in keypoint tracking applications. As a rule of thumb, given an approximately 10 mm span between many key landmarks on a mouse (Ramalingasetty et al., 2021), the inventors assume that the average error of a given keypoint tracker should not exceed 2.5 mm, which is approximately 6.1 pixels given the optical setup. Here, the inventors consider error to be the average L2 distance between the network-identified keypoint and the corresponding Qdot fluorescence peak.

Training and testing U-Nets were used to identify the location of Qdots, and the body part with which each injection was associated (e.g., right hindpaw, left forepaw, tip of the tail) given only surface images of the mouse. U-Nets were experimented using frames from all camera views (“all”), training on four views and testing on the one held out view (“different”), and training and testing using frames from the same camera view (“same”) (FIGS. 41 and 4J, FIGS. 17-18). When training and testing using frames from the same camera view, 450 frames were required to achieve a mean error of 2 mm. More frames were required for the other two scenarios: 850 frames using all camera views, and 17,250 frames when attempting to generalize to a novel view. To achieve submillimeter accuracy, 25,550 frames were required for the same camera view, and 85,250 frames using all camera views, (FIG. 4K). Relative to the discrepancy between human labelers, depending on the body part, the method outperforms human labelers by up to 1.5 to 5.2-fold (FIG. 16).

### OD800 can be Conjugated to Antibodies for Targeting Specific Proteins

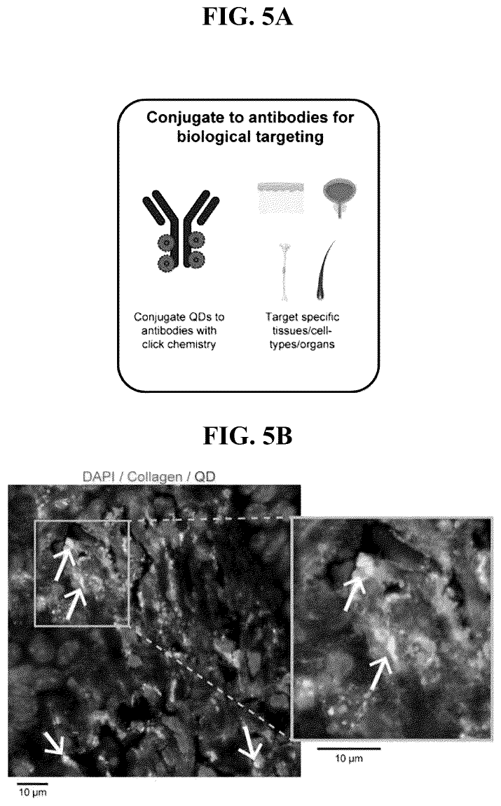

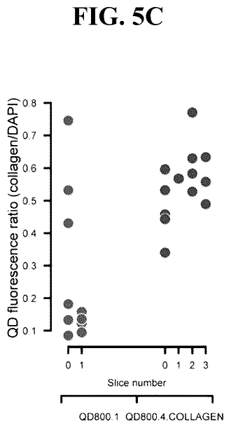

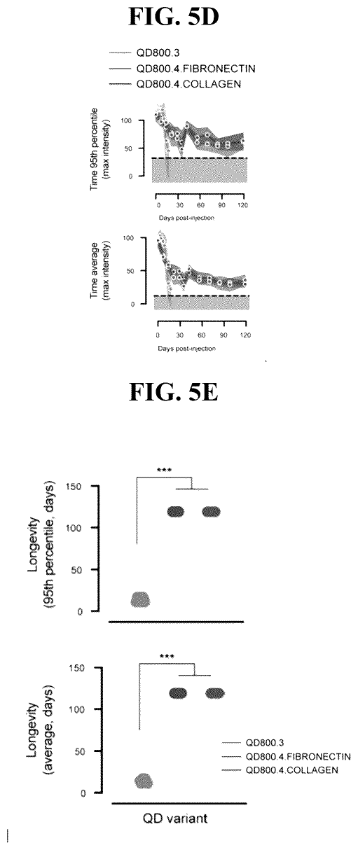

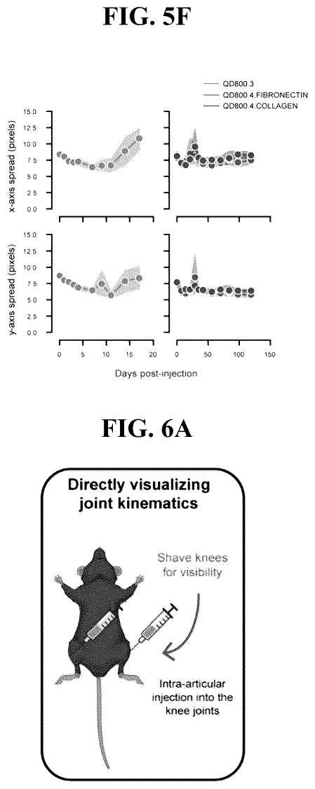

Having established QD800.3's enhanced performance in labeling adipose tissue, it was speculated that quantum dots could also be deployed in applications that required additional biological specificity; that is, quantum dots could be used in applications that required highlighting specific proteins, cell types, or tissues. As a proof of principle, a protocol was adapted for conjugating quantum dots to antibodies via click chemistry (see Methods). Here, the experiments were tested directly conjugating quantum dots to antibodies that target ultra-long-lived proteins in the extracellular matrix in skin and joints: collagen and fibronectin (QD800.4.COLLAGEN and QD800.4.FIBRONECTIN, (FIGS. 5A, 5B, and 5C).

To visualize the dynamics of these quantum dots in the skin, as an example, the inventors performed QD800.4.COLLAGEN injections along the back of an additional cohort of animals and harvested the tissue for histology (n=2 tissue samples/injections, n=1 with QD800.1 as a control and n=1 with QD800.4.COLLAGEN, see Methods). Using immunofluorescence (IF), the tissue sections were stained with Collagen-I antibodies and a cell nucleus marker and confocal microscopy was used to simultaneously visualize QD-related fluorescence and collagen-expression-related fluorescence in multiple fields of view (FIG. 5B). The images revealed that QD800.4.COLLAGEN was more likely to colocalize with collagen-I relative to QD800.1. To quantify that, the ratio of quantum dot fluorescence was commuted in collagen-I-rich regions to cell nuclei. The inventors found that QD800.4.COLLAGEN had a median ratio of 0.56 and QD800.1 had a median ratio of 0.15 (FIG. 5C).

Remarkably, it was found that both QD800.4 variants outperformed QD800.3, with resolvable fluorescence up to 119 days post-injection in all animals (FIGS. 5D and 5E). Moreover, the fluorescence spread of both was reviewed and compared to QD800.3 and they found that QD800.4's shape remains stable for as long as the inventors were able to image it in vivo (FIG. 5F).

### OD800 can Directly Track Joint Kinematics

Finally, it was speculated that, since QD800.3 yielded bright, long-lasting discrete points of fluorescence under the skin, could they introduce QD800.3 directly into the knee joint and still acquire a resolvable signal. That would confirm that the method can provide direct visualization of key body parts for precisely quantifying kinematics in mice.

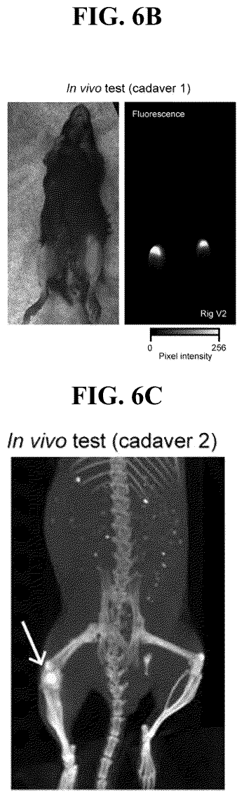

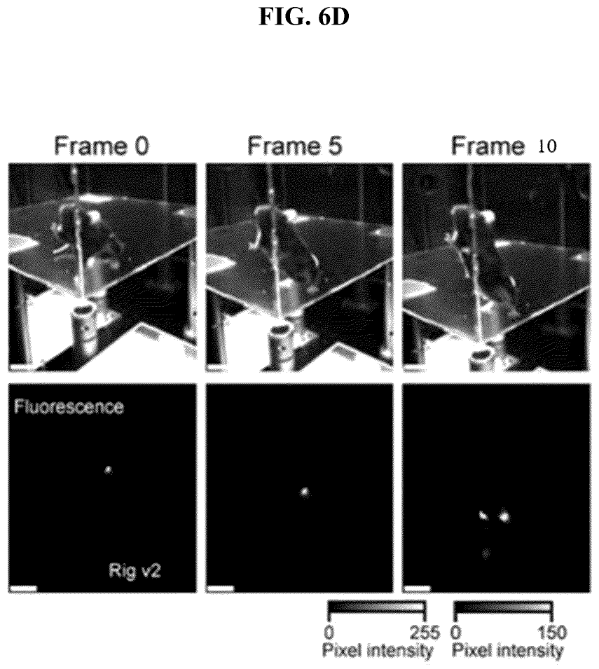

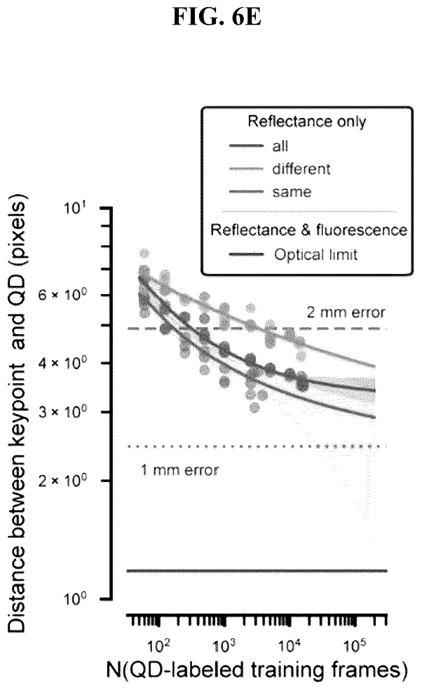

Guided by an existing protocol (49), the quantum dot injection protocol was altered to allow for intra-articular injection of QD800.3 into both knee joints in a mouse (FIG. 6A). The first test was performed on a mouse cadaver (FIG. 6B). With each quantum dot injection, it was confirmed that movement of the overlaying skin did not affect the fluorescent spot using a custom-built NIR-camera setup (see Methods). The location of the injection was further confirmed in relation to the skeleton with a micro-computed tomography (microCT) image from a mouse cadaver using IVIS Spectrum CT (Perkin Elmer/Revvity; FIG. 6C, see Methods). Additionally, intra-articular injections were performed into the knee joints of live mice and subsequently imaged them in the plexiglass arena (1-hour post-injection, FIG. 6D). It was confirmed that the intra-articular injection sites could be reliably tracked as the mice moved freely

Given the high SNR of QD800.3 directly injected into the knee joints, it was hypothesized that a markerless keypoint tracker could be trained to accurately track the position of the joints. To test this, a small number of frames were hand labeled (n=872 frames from n=1 mouse) to map each fluorescent point to the corresponding body part (left or right knee joint). These labels were then used to train a U-Net to leverage reflectance and fluorescence data to label the full dataset. Finally, this dataset was used to benchmark the ability of the trained U-Net to track the position of the knee joints given surface features (FIG. 6E). Here, it was found that 2 mm accuracy could be achieved using 250 frames for the same camera view, 350 frames for all camera views, and 3350 frames when generalizing to a novel view. The number of frames required for 1 mm accuracy exceeds the maximum number of frames that the inventors tested (n=17,901 frames).

To confirm the targeting efficiency of the knee injections, the experiments were repeated in a second cohort of animals following the same protocol (n=4 mice, see Methods). Seven out of eight knee injections were successful on the first attempt, while one knee injection required a second attempt due to glass pipette breakage

### DISCUSSION

Here, a method was developed called QD-Pi for optically tracking movement in freely moving mice. Importantly, it is shown that quantum dots can be injected into joints and used to directly image joint kinematics. This is a key step toward measuring skeletal movement in animals, and it distinguishes QD-Pi from markerless keypoint tracking methods, which are limited to tracking points on the surface of an animal.

To establish quantum dots as optical tags, NIR-I-emitting quantum dots (QD800) were injected, free or immobilized on microbeads, in discrete spots in the adipose tissue just beneath the dermis of each animal. It was found that the microbead-immobilized particles formed long-lived deposits that can be used as fiducials for tracking keypoints inside the body using standard machine vision cameras. It was also found that two commercially available variants were suitable for in vivo use: quantum dots originally designed for tracking the vasculature (QD800.1) and quantum dots tailored for cell labeling (QD800.2). However, the fluorescence of both variants decayed to noise levels within 1-5 days after injection. Next, a custom third variant was designed where biotinylated quantum dots are attached to porous, streptavidin-functionalized agarose microbeads (QD800.3). QD800.3 led to robust labeling with fluorescence that remained resolvable for over 2 weeks after injection. A variant (QD800.4) was then developed that can be customized for biological targeting, which the inventors used to target quantum dots to long-lived proteins in the extracellular matrix. Signals from QD800.4 remained resolvable for as long as the inventors imaged it, up to 4 months post-injection, a timescale readily compatible with longitudinal systems neuroscience experiments. Quantum dot localization was characterized in vivo and show that the QD800.2 signal is predominantly intracellular compared to QD800.1, which is mostly deposited outside the cell. Additionally, the analysis shows that QD800.4.COLLAGEN is more likely to colocalize with collagen-I compared to QD800.1. Furthermore, it was demonstrated that quantum dots can be resolved in vivo using substantially different excitation setups. Longer wavelengths may be more desirable in some experimental setups (e.g., to minimize interface with other imaging equipment), while shorter wavelengths enable higher fluorescence levels. Lastly, it was shown that quantum dots can be used to directly visualize joints, a key step in accurately resolving kinematics in mice.

In this study, the inventors tested quantum dot variants in freely moving mice in an open arena. That presents additional challenges in quantification due to variations in excitation power as mice change positions relative to the excitation LEDs. Nevertheless, the method of quantification computes fluorescence as mice occupy different positions in the arena, across five different camera views, and across multiple mice. Additionally, the inventors show that quantum dots can work with substantially different hardware configurations (Rig vl as seen in FIG. 7 compared to Rig v2), indicating that the method is robust to variation in excitation and emission optics; that means downstream users of QD-Pi can tailor optical hardware to their particular needs.

Quantum dots represent an attractive alternative to other tags employed in marker-based methods that have been used in rodents. Elegant work from Butler et al. demonstrated that UV ink can be applied to the fur and used to measure a single point in freely moving mice (50) (see also (26) for a similar method using non-fluorescent paint applied to rodents). However, UV excitation light may be visible to the animal (48), the ink can fade, and similar markings have been shown to be relatively imprecise (17). Since the ink is applied to the outer layer of the skin and fur, it can also be groomed or licked off by the mouse. Other studies have used piercings with highly reflective markers that can be tracked using commercial motion capture systems originally designed for use in humans (17, 51). However, these markers are bulky and still represent motions on the surface of the skin. Another marker-based work from Moukarzel et al. applied tattoo ink directly to the tissue surrounding the knee joint of rats (52). Although this method targets the musculoskeletal system, it appears to have relatively low SNR since the tattoo ink is simply dark under IR illumination rather than fluorescent, the ink likely disperses widely around the joint preventing precise localization, and it has only been shown to mark a single joint at a time.

As an alternative to marker-based methods or X-rays, markerless keypoint trackers have capitalized on recent developments in image-based deep learning methods, resulting in a surge of interest in movement tracking (5-10). However, they often require laborious hand-labeling of training data, which leads to imprecise predictions due to inter-labeler jitter and ambiguity in labeling certain keypoints (14, 17). The method also requires hand-labeling, but only of an initial dataset to train a markerless keypoint tracker to identify fluorescence spots. That enabled the creation of a large dataset with reference point labels for keypoint trackers—that is, labels with well-defined physical positions inside the body-which is called QD-Pi-120K. Typically, labels are identified by humans on surface images of the mouse; here, fluorescence spots provide physical markers of body part locations for unambiguous labeling. Using the resulting dataset, it was found that only hundreds of frames were required to achieve an acceptable threshold of 2 mm average error with markerless keypoint trackers, in line with prior work (10, 50). However, human kinematics studies typically track kinematics with submillimeter precision, and humans are on the order of 20 times larger than laboratory mice; thus, the relative error of kinematic tracking in mice is still much higher relative to studies of movement in humans. Achieving submillimeter average error or less in the hands requires at least 25,550 labeled frames, with at least 85,250 required if one wants a single network that works across multiple views, or more than 100,000 to generalize to a novel view. Datasets of this size are not feasible with manual labeling, which emphasizes the critical need for techniques like ours that enable the collection of large, high-quality datasets for training the next generation of markerless keypoint trackers. Moreover, in addition to training markerless keypoint trackers, quantum dots can be used directly for high-resolution kinematic tracking without these steep requirements on the amount of training data.

Despite the impressive performance and ease-of-use of markerless keypoint trackers, these methods fundamentally rely on surface features of the animal. Prior X-ray videography of freely moving rodents has shown that, even if one can optimally track keypoints on the skin, the skin can distort the movement of the underlying joint (22). While quantum dot optical tags were established in the adipose tissue, experiments also demonstrate intra-articular quantum dot injections. Direct joint tracking via QD-Pi is a promising approach to accurately measure the part of the body under more direct control of motor circuits—the muscles, joints, and bone; that will allow us to more comprehensively map the relationship between neural activity and movement.

However, while using quantum dots as optical tags is an attractive approach, there are limitations. Signals from QD800.1 and QD800.2 rapidly faded with a timescale of 1 and 5 days, respectively. Prior work has speculated that QD800.2 may quickly leak from cells either through excretion or cell division (53, 54), which could explain the histology results, where the inventors noted that some QD800.2, although less than QD800.1, was deposited outside cells. That said, signals from QD800.4 versions that targeted long-lived extracellular matrix proteins lasted up to 4 months. Using QD800.4.COLLAGEN as an example, the inventors show that QD-conjugated antibodies may colocalize with low-turnover proteins of interest. It is important to note that the efficacy and reliability of such QD-conjugated antibodies are heavily dependent on the binding specificity, affinity, and permeability of a given antibody to bind its intended target.

Additionally, resolving signals from QD800 injections in the fatty tissue along the back required shaving the fur. This is subject to the same limitations faced by other agents used in noninvasive biomedical imaging (55-59), as either nude mice are needed, or fur is removed at the imaging region. Moreover, the resolution of the signals also suffered from crowding in certain body parts. While experiments were able to further analyze and partially account for the spread of the fluorescent points (FIG. 15), since the quantum dot solutions are injected by hand, experimental control over particle spread is limited. That may be addressed in future work by performing slow, controlled injections via a syringe pump or other automated micro-injection system. That would also provide more precise control over injection volume, which should allow for precise control over fluorescence spot size to prevent signal crowding. Another future direction is to explore more sophisticated peak-finding algorithms, including mean-shift or mixture of Gaussian-based clustering.

Another limitation is that the mechanism of quantum dot clearance from the body remains an open question, including for the microbead-immobilized quantum dots presented here. It has been reported that quantum dots that were administered intravenously and intradermally are deposited in the liver, lymphatic, and renal systems of mice and rats (45, 60, 61). Additional investigation into this question may provide insight into further improvements in half-life and signal longevity.

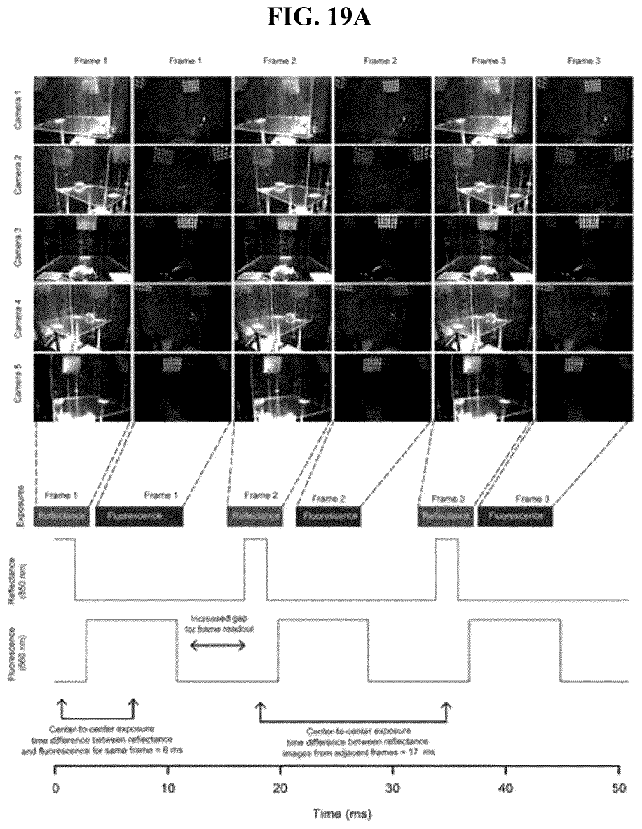

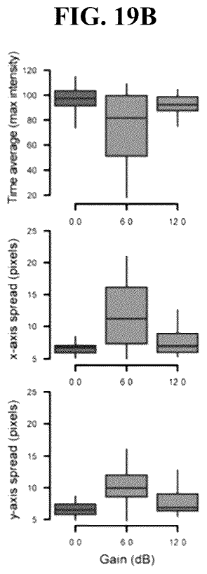

It is noted that the dataset generated by the system, QD-Pi-120K, is only an initial step to generating high-quality keypoint tracking datasets at scale. In the future, the plan is to further improve the SNR and time resolution of the system. The speed of the current multiplexed imaging system, approximately 30 frames per second, with 17.5 milliseconds between the reflectance and fluorescence exposures. Thus, there is the possibility of minor displacements between the fluorescence data and surface features in QD-Pi-120K during periods of rapid motion. While experiments were able to successfully reduce this delay to 6 milliseconds without substantial loss of SNR (FIG. 19), subsequent versions of the imaging rig will focus on further reductions in time between these exposures to minimize the possibility of that occurring and to increase the effective frame rate. As the imaging speed of the system and SNR is further optimized, in the future, the inventors plan on generating additional benchmark datasets that improve on QD-Pi-120K. Furthermore, with future improvements, the inventors plan on directly characterizing the performance of QD-Pi for capturing body kinematics during ethologically relevant behaviors in mice. That includes locomotion (62), grooming (4), and paw trajectories (63).

Additional future directions include injecting quantum dots directly into the muscle, bone, and specific tissues within a joint, such as the tendons. Recent developments of fluorescent dyes in NIR-I (55) and shortwave infrared (SWIR, 1000-2000 nm) (56-58) range have made it possible to visualize deeper tissue structures, like the skeleton or organs, and perform multiplex imaging in both anesthetized and awake mice through intravenous injections. While the inventors chose to use quantum dots in the NIR range to facilitate the adoption of the technique, future studies should look at incorporating quantum dots in the SWIR range, as it may have higher signal-to-noise, especially for deep-tissue injections (59).

Another direction is to label other tissues like the whiskers, and potentially internal organs. While the plan to systemically characterize which of the quantum dot variants works best for targeting various body parts, highly specific targeting to cell types, tissues, and molecules will be enabled by further variations on QD800.4. Moreover, quantum dots have near-ideal optical properties for spectral multiplexing. Quantum dots are available at a wide variety of emission wavelengths spanning most of the visible spectrum into mid-IR with high quantum yields (64, 65), with conveniently overlapping excitation spectra. Consequently, one could easily label separate mice or separate parts of the body with distinct colors.

Lastly, the markerless keypoint trackers used here are relatively simple models based on the U-Net architecture (11). There are now much more complex architectures, e.g., transformer networks (66), that could better capitalize on the large datasets that can be produced by the system. Additionally, the inventors did not systematically explore bottom-up or top-down architectures, each of which could lead to substantial performance gains.

Thus, the inventors present a method for directly tracking positions inside an animal's body. In addition to advancing the measurement of kinematics in freely moving mice, the inventors envision that the method can be used to augment markerless keypoint trackers, which have become the standard method for movement tracking in laboratory animals (5-10). By imaging both the fluorescence from specific points in the body and the surface features surrounding those fluorescent points simultaneously, the inventors have amassed a large dataset that can be used to benchmark models for tracking keypoints in 2D (5, 7, 9, 10) and 3D (6, 8). Moreover, in addition to acting as a method for directly measuring the motion of positions inside a mouse's body, the method will be instrumental in building reference point datasets at the scale necessary for constructing foundation models for tracking motion in laboratory animals. Ultimately, this method can be used to resolve complex motor patterns in laboratory organisms with newfound accuracy and precision.

### Figure Descriptions

FIG. 1 refers quantum dots (Qdots) that can act as optical markers when introduced into the skin. FIG. 1A is a schematic representation of the quantum dot injection procedure. FIG. 1B Schematic representation of the basic optical properties of NIR-emitting quantum dots. FIG. 1C is histological examination of quantum dots injected into the back of a mouse. Top, schematic of the experiment. Injectable quantum dots were introduced into the skin, after which tissue samples were harvested for imaging. Bottom left, example fluorescence image of a skin sample after quantum dot injection (QD800.2). Mean projections are shown along the x- and y-axes. Bottom right, fluorescence probability densities at different tissue depths aligned to the tissue boundary (left) or the fluorescence peak (right). Each plot corresponds to a separate injection (QD800.1 or QD800.2). Full width at half maximum is shown next to each distribution. FIG. 1D is a schematic of the plexiglass arena and optical configuration for in vivo imaging. Reflectance and fluorescence images are collected near-simultaneously using IR-emitting LEDs and polarized NIR-I-emitting LEDs, respectively. Five machine vision cameras equipped with long-pass and polarization filters are used to collect images. FIG. 1E is an example reflectance and fluorescence images from vehicle (left) and QD-injected mice (QD800.2, right). Scale bar indicates 50 pixels. FIG. 1F is a schematic illustrating how quantum dot fluorescence is measured. First, the max intensity is computed per frame, then either the 95^{th} percentile or the mean is computed across all frames for each mouse and camera. FIG. 1G is a 95^{th} percentile pixel intensity comparison of vehicle/blank (n=6 mice) and quantum dot mice (n=3 QD800.1 and n=3 QD800.2 mice) (p=0.002, U=36, f=0, Mann-Whitney U test). Measurements were averaged across the five cameras. FIG. 1H is the average pixel intensity comparison between vehicle/blank or quantum dot injection (p=0.002, U=36, f=0, Mann-Whitney U test).

FIG. 2 describes the half-life of quantum dots in buffer (QD800.1) and quantum dots that enter cells (QD800.2). FIG. 2A is a schematic of quantum dot injection sites with QD800.1 and QD800.2 and their hypothesized aggregation in tissue. QD800.1 likely resides in the extracellular matrix, while QD800.2 is designed by the manufacturer to enter cells. FIG. 2B is the reflectance and fluorescence image of a mouse immediately after injection of QD800.2. FIG. 2C is example fluorescence images from QD800.1 (top) and QD800.2 (bottom) imaged at 0 and 2 days post-injection. FIG. 2D is an example confocal images from injections into the backs of mice with either QD800.1 (top) or QD800.2 (bottom). Tissue was stained with DAPI (for nuclei) and WGA (for cell membranes). FIG. 2E is the ratio of quantum dot fluorescence inside of cells to outside of cells (p=0.015, f-0.85, Mann-Whitney U test; n=9 fields of view from n=2 tissue samples/injections for QD800.2 and n=8 fields of view from n=2 tissue samples/injections for QD800.1). FIG. 2F is either 95^{th} percentile (top) or average pixel intensity (bottom) plotted as a function of days post injection for both variants (see FIG. 1F for schematic of quantification). Shown is the mean (line) and one standard deviation (shaded region) across each quantity for every mouse/camera view pair (n=15). Gray region indicates 99^{th} percentile confidence interval for vehicle/blank mice. Same conventions used throughout. FIG. 2G is the initial brightness (top) and the fluorescence longevity (time for the trace for each mouse/camera pair to cross below the 99^{th} percentile of vehicle/blank mice, bottom) for both variants calculated from 95^{th} percentile pixel intensity over time (p-0.23, U=142, f-0.63 for brightness; p=6.9e-6, U=219, f=0.97 for longevity, Mann-Whitney U test; n=15 mouse/camera pairs each). FIG. 2G is the same as FIG. 2G, except computed using average pixel intensity over time (p=0.41, U=133, f=0.59 for brightness; p=7.5E-5, U=204, f=0.91, for longevity, Mann-Whitney U test; n=15 mouse/camera pairs each). FIG. 2I is a schematic of spatial autocorrelation calculation to measure the length-scale of QD-induced fluorescence. FIG. 2J is the average in vivo spatial correlation of fluorescence across all mice and camera views for both QD800.1 (top) and QD800.2 (bottom).

FIG. 3 describes a custom agarose bead quantum dot mixture (QD800.3) that outperforms all other variants in vivo. FIG. 3A is a schematic representation of quantum dot injection sites with QD800.3. FIG. 3B is Brightfield (left) and fluorescence (right) images from the same tissue sample taken from the back 1 day after QD800.3 injection. Scale bar represents 500 μm. FIG. 3C is example fluorescence images from QD800.2 (top) and QD800.3 (bottom) imaged at 4 days post-injection. Scale bar indicates 50 pixels. FIG. 3D is either 95^{th} percentile (top) or average (bottom) pixel intensities plotted as a function of days post-injection for all three variants. Line indicates the average and shaded region one standard deviation across mouse/camera pairs. FIG. 3E is the initial brightness (top) and the decay rate (bottom) for all variants computed using the 95^{th} percentile across time (p=0.013/0.19, U=173/144.5, f=0.77/0.64 for brightness; p=2.5E-6/2.3E-6, U=225/225, f=1/1 for longevity, Mann-Whitney U test; n=15 mouse/camera pairs each, QD800.3 compared with QD800.1/QD800.2). FIG. 3F is the same as FIG. 3E, except computed using the average across time (p=0.004/0.009, U=182/176, f=0.81/0.78 for brightness; p=2.7-e6/2.5E-6, U-225/225, f=1/1 for longevity, Mann-Whitney U test; n=15 mouse/camera pairs each, QD800.3 compared with QD800.1/QD800.2). (G) Spatial autocorrelation for all three variants in vivo.

FIG. 4 describes combining QD800.3 and an optimized imaging rig that enhances SNR and enables scaling of markerless keypoint trackers. FIG. 4A schematic of changes to optimize the protocol. FIG. 4B is fluorescence images from mice injected with QD800.3 using Rig v1 as seen in FIG. 7 (top) or Rig v2 (bottom). Scale bar indicates 50 pixels. FIG. 4C is the SNR across all variants and rigs, estimated by taking the standard deviation across x- and y-axes for each fluorescence frame and then averaging the result across all frames for each mouse/camera view pair (QD800.3 Rig v2 comparison with all other variants, p=3.4E-6, U=0, f=1, Mann-Whitney U test, n=15 mouse/camera pairs). The noise level was estimated by calculating the same metric for blank/vehicle mice. FIG. 4D is a Schematic illustrating scaling up of labeling (see Methods). FIG. 4E is keypoint prediction localization error relative to hand-labeled points using reflectance images (“reflectance only”), fluorescence images (“fluorescence only”), or their combination (“both”). Errors are averaged over all keypoints and frames for a model trained with a new random seed (n=5 restarts). FIG. 4F is the performance of the “both” model after subsampling the training dataset. FIG. 4G is the average held-out error for a model trained using a different random seed (n=5 restarts) with and without quantum dot fluorescence refinement (see Methods). FIG. 4H shows for each frame, the difference in pixel error is shown with and without fluorescence refinement. FIG. 4I is the schematic for using machine-labeled frames to train markerless keypoint trackers on reflectance data only. FIG. 4J shows how U-Nets were trained and tested using data from the same camera view (“same”, top), all camera views (“all”, middle), or trained on four camera views and tested on one held out view (“different”, bottom). FIG. 4K is the results from training U-Nets according to FIG. 4J. Error is the distance between the prediction and the nearest quantum dot fluorescence peak. For model performance curves, lines reflect the median power-law decay fit and shaded region indicates 95% bootstrap confidence interval.

FIG. 5 describes targeting specific tissues with QD800.4 for substantially increased longevity. FIG. 5A describes the strategy for conjugating QD800 to antibodies in order to target specific tissues, cell types, and molecules. FIG. 5B shows, Left, example confocal image from QD800.4.COLLAGEN injection into the back. Tissue was stained with DAPI (for nuclei) and collagen to assess localization. Right, zoom-in of the region-of-interest indicated by the bounding box in the left image. FIG. 5C is the ratio of quantum dot fluorescence colocalized with DAPI to collagen (n=13 fields of view from n=1 tissue sample/injection for QD800.4 and n=10 fields of view from n=1 tissue sample/injection for QD800.1). FIG. 5D is the fluorescence plotted against days post injection with same plotting convention as FIGS. 2F and 3D. FIG. 5E is the quantification of longevity of fluorescence signal per mouse/camera pair using the 95^{th} percentile across time (p=1.5E-5, U=0, f=0 for longevity comparing QD800.3 to QD800.4.COLLAGEN, Mann-Whitney U test; p=1.5E-5, U=0, f=0 comparing QD800.3 to QD800.4.FIBRONECTIN; n=15 mouse/camera pairs for QD800.3, n=10 mouse/camera pairs for each QD800.4 variant, and n=10 vehicle-injected mouse/camera pairs) and using the average across time (p=1.3E-5, U-0, f-0 for longevity comparing QD800.3 to QD800.4.COLLAGEN, Mann-Whitney U test; p=1.3E-5, U=0, f=0 comparing QD800.3 to QD800.4.FIBRONECTIN). Note that longevity for QD800.4 was as long as they were imaged. FIG. 5F is the spread of fluorescence in x- and y-axes for QD800.3 (left) and QD800.4 (right) over time, assessed using the spatial autocorrelation (see Methods).

FIG. 6 describes the direct visualization of the knee joint in vivo through instar-articular injection of QD800.3. FIG. 6A shows QD800.3 was injected directly into the knee joints after shaving fur. FIG. 6B shows in vivo cadaver validation of intra-articular targeting. FIG. 6C shows micro-computed tomography (microCT) image from the right knee joint of mouse cadaver. Fluorescence is overlaid (yellow spot) on top of a reconstructed mouse skeleton. Arrow indicates location of fluorescence spot. FIG. 6D shows Frames from video recorded 1 hour post-injection into live mice. FIG. 6E use the same procedure as FIG. 4J, where the inventors trained keypoint models to track knee joint location based on surface features. Plotting conventions are the same as FIG. 4J.

FIG. 7 describes the quantum dot imaging rig version 1. What is shown in FIG. 7A is the rig and plexiglass arena used for imaging quantum dots (quantum dots) in freely moving mice. All data included in FIGS. 1, 2, and 3 and FIGS. 8, 9, and 10 were collected using this setup. FIG. 7B is a schematic of the illumination sequence used for temporal-division multiplexing. Note that the fluorescence images presented here are not background-subtracted.

FIG. 8 describes fluorescence data pre-processing. FIG. 8A shows that to remove static elements from the scene (e.g., excitation LEDs), the background of the image was computed using a non-overlapping 1500-frame-long sliding window. All visualization and quantification of fluorescence data uses background-subtracted data. FIG. 8B shows the quantification of fluorescence intensity and longevity in FIGS. 1, 2, 3, 4, and 5 used peak fluorescence across the entire frame and subsequently computed either the 95^{th} percentile or the average across frames. To verify that non-mouse pixels did not contribute meaningfully to these estimates, the inventors manually labeled 1,395 reflectance images from all five cameras to specify which pixels were occupied by the mouse. Then, the inventors trained a Segformer using the nvidia/mit-b3 variant with pretrained weights from Huggingface. Example segmentation masks and the corresponding reflectance images are shown. FIG. 8C shows Either average or 95^{th} percentile of max intensity computed using either the full frame or the mouse ROI. FIG. 8D is the analysis in FIGS. 1G and 1H repeated using the peak fluorescence computed with the mouse ROI rather than the full frame. Analysis in FIG. 2F-H repeated using the peak fluorescence computed with the mouse ROI rather than the full frame.

FIG. 9 describes an example of reflectance and fluorescence data from a single session. FIG. 9 shows reflectance (left) and fluorescence data (right) for four frames-timestamps are given at the top—from all five hardware-synchronized cameras. Data from a mouse injected with QD800.2

FIG. 10 describes an example of reflectance and fluorescence data from a single session. FIG. 10 uses the same layout as FIG. 9, except a different session is shown. The Data from a mouse injected with QD800.2. The mouse shown here is different from the mouse shown in FIG. 9.

FIG. 11 describes extending the longevity of quantum dot fluorescence by attaching the quantum dots to microbeads: FIG. 11A shows a droplet from six different microbead brands (Amid Biosciences, #SA-101-1; Bangs Laboratories, #CP01008; Resyn Biosciences, #MR-STM002; PolyAn, #105-21-020; Thermo Scientific, #20357; Vector Laboratories, #N-1000-002) mixed with biotinylated quantum dots imaged under brightfield (top) and fluorescence (bottom) using Cy7 excitation/emission (see Methods for details). Scale bar represents 500 μm. FIG. 11B shows a schematic of attaching quantum dots to a relatively large, biocompatible, porous agarose beads will lead to stabilization of quantum dot fluorescence. the inventors call this QD800.3. FIG. 11C is protocol for QD800.3. Streptavidin-coated agarose beads are mixed with biotinylated quantum dots and incubated for 1 hour at 40° C., mixed halfway through the incubation period. The supernatant is removed, the solution is washed 3 times with 1×PBS, and the beads are resuspended in 2% sodium alginate. FIG. 11D is images of agarose beads (top) and the agarose bead quantum dot mixture (bottom). Images were taken under 405 nm illumination. FIG. 11E is a drop et of agarose beads (top) or the agarose bead quantum dot mixture (bottom) under brightfield illumination. Scale bar represents 500 μm. FIG. 11F is fluorescence from the same droplet using Cy7 excitation/emission (see Methods for details). Scale bar represents 500 μm. FIG. 11G is the pipette tips for quantum dot injections. Each minor division is 10 μm . . .

FIG. 12 describes histology from a left forepaw injection of QD800.3 (agarose bead quantum dot mixture). FIG. 12 shows, left, schematic of experiment. QD800.3 was injected into the left forepaw, then tissue was harvested 1-week post-injection and imaged, middle, brightfield image of injection site, and right, fluorescence image of the injection site. Scale bar represents 500 μm.

FIG. 13 describes quantum dot imaging rig version 2. FIG. 13A shows the setup used to collect all data included in FIGS. 4, 5, and 6 and FIGS. 14, 15, 16, 17, 18, and 19. FIG. 13B shows quantum dots that were pipetted onto a slide and imaged in the plexiglass arena once every 30 seconds for 24 hours to measure photostability in rig version 2. Excitation light was left on continuously. Top, an ROI was selected around the slide, and the peak was computed over the ROI per frame. Shown is the peak intensity in the image ROI and the peak intensity of a blank patch of the same frames. Bottom, similarity of the ROI to an average of the last ten frames. Due to drying, the quantum dot droplet rapidly changes shape over the first hour. Once the spatial distribution of fluorescence is stable, the quantum dots are highly photostable.

FIG. 14 describes an example of reflectance and fluorescence data from a single session collected using rig version 2 using the same layout as FIGS. 9 and 10. The mouse shown here was injected with QD800.3.

FIG. 15 describes the spatial profile of quantum dot fluorescence and its impact on marker localization. Using keypoint predictions from the “reflect+fluo” model used in FIG. 4E, 4F, and 4G, the inventors took a 30×30-pixel window around each predicted keypoint location, and averaged windows across all predictions for each body part. For computational efficiency, the inventors randomly sampled 2,000 windows for each keypoint. Here, “Back” combines the bottom, middle, and top back keypoints; “Paw” combines all paw keypoints, and “Tail” combines the base, middle, and tip of tail keypoints. Each row shows the fluorescence after using a different method for refining the keypoint prediction using fluorescence: original, no fluorescence refinement; COM, using only the center of mass; COM+cleanup, a contour was fit to the fluorescence close to the predicted keypoint, pixels not inside of the contour were masked out prior to computing the center of mass; Gauss., fitting a bivariate Gaussian to the fluorescence data and using the mean to detect the center; Gauss.+cleaning, using a contour fit same as COM+cleanup and then fitting a Gaussian to the pixels after applying the cleanup mask. FIG. 15B is a histogram of the intensity data shown in FIG. 15A using radial bins. FIG. 15C is the quantification of the data shown in FIG. 15A. Left, boxplot of the peak pixel intensity at the center of the cropped window. Right, distance from the center to reach 50% of peak intensity. FIG. 15D shows that to assess the impact of fluorescence intensity and the spread of fluorescence on detection of the center of fluorescence, the inventors performed simulations where the inventors varied intensity, spread of fluorescence, and spread aspect ratio (ratio of spread along the x-axis to spread along the y-axis). the inventors varied each of these three parameters and performed 100 simulations per parameter setting. In each simulation, the inventors used the algorithm for detection the center of fluorescence (Gauss. +cleanup from FIG. 15A) and calculated how far the detected center was from the true center. Here, the inventors show performance across all three parameters. Min projection indicates that, for the parameter not shown in the heatmap, the inventors compute the minimum error. Max projection indicates that, for the parameter not shown in the heatmap, the inventors compute the maximum error. FIG. 15E is filled contour plot of the simulations shown in FIG. 15D. FIG. 15F shows that to determine the effect of payload volume on fluorescence parameters, the inventors repeated injections of QD800.2 at three volumes (2.0 μL is the original volume used for FIGS. 1, 2, and 3). Here the inventors show boxplots of the spread of fluorescence along the x-axis and y-axis, quantified by calculating the standard deviation along the x- and y-axes from a Gaussian fit to the spatial autocorrelation profile (n=2 mice per injection volume viewed from n=5 cameras, so n=10 camera/view pairs per injection). FIG. 15G is boxplots of aspect ratio per injection volume. FIG. 15H is boxplots of time average of peak fluorescence intensity per injection volume. Gray region indicates 99^{th} percentile from vehicle injected mice (same mice as in FIG. 5D).

FIG. 16 describes assessing the accuracy of manual annotation with and without quantum dot fluorescence FIG. 16A is histograms of maximum pairwise distance between n=4 labelers who each annotated the same n=271 reflectance frames with the 10 keypoints shown in FIG. 4D, and an additional n=274 reflectance frames with the 2 knee joints. Shown is the maximum pairwise distance across labelers per keypoint/frame pairs for the back keypoints, paw keypoints, tail keypoints, and knee joints. Vertical lines and associated numbers are the median of each distribution. In red is the median of the top 5 SLEAP models trained to predict keypoints from all camera views (“all” from FIG. 4K or FIG. 6E). FIG. 16B shows, Top, histograms of discrepancies across labelers for reflectance frames (blue), and the discrepancy between labelers and quantum dot fluorescence centroids with frames with alpha-blended fluorescence (orange). Bottom, cumulative histograms of the same data shown on top. FIG. 16C Boxplots of pairwise errors across labelers for reflectance frames along with the discrepancy between labelers and quantum dot fluorescence centroids for each of 6 different labelers.

FIG. 17 describes assessing the accuracy of reflectance and fluorescence-based keypoint detection at different positions in the arena. The 5-camera system was calibrated using a set of 4 ChArUco boards arranged into a cube. The cube was manually spun while the inventors collected reflectance exposures. Next, OpenCV was used to estimate the intrinsics and extrinsics of each camera. Bundle adjustment was performed to optimize extrinsics with respect to reprojection error using standard technique (see Methods). Then, using the model that predicted keypoints using both fluorescence and reflectance data from FIG. 4E, 4F, and 4G, the inventors triangulated fluorescence-refined keypoints to estimate the x,y,z position of the mouse and then calculated the distance between predicted keypoints and fluorescence centroids as a function of position in the arena. FIG. 17A shows, Left, the median distance between predicted keypoints and fluorescence centroids shown as a function of distance from each camera and the viewing angle. Right, median distance computed across all cameras. FIG. 17B is the distance between keypoints and quantum dot fluorescence centroid shown as a function of position in the arena. First, for each keypoint, the inventors calculated the error (distance between predicted keypoint and fluorescence centroid) for each camera. Next, the inventors kept the error from the camera that was closest to the centroid of the mouse. Finally, the inventors show the 0.25, 0.5, and 0.75 quantiles of the distribution of errors at each position in the arena.

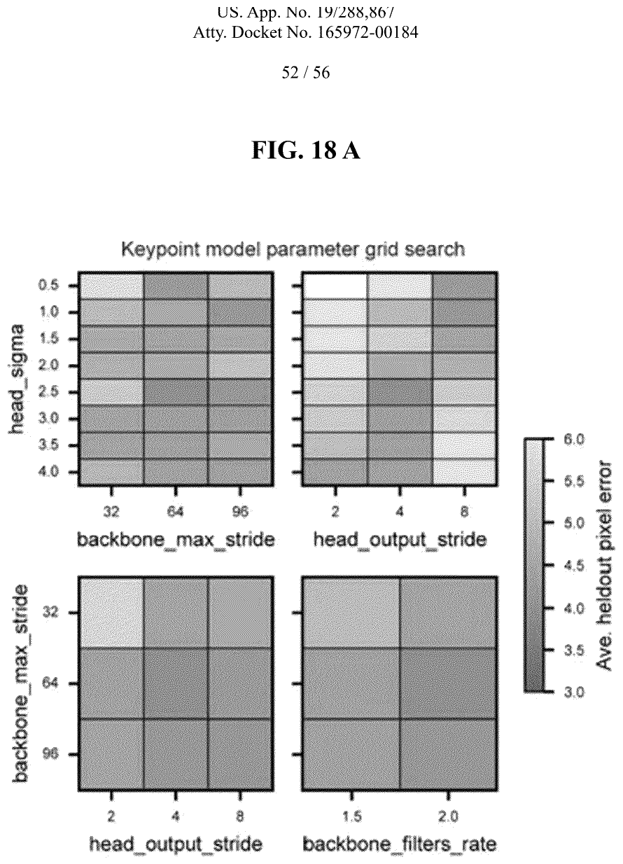

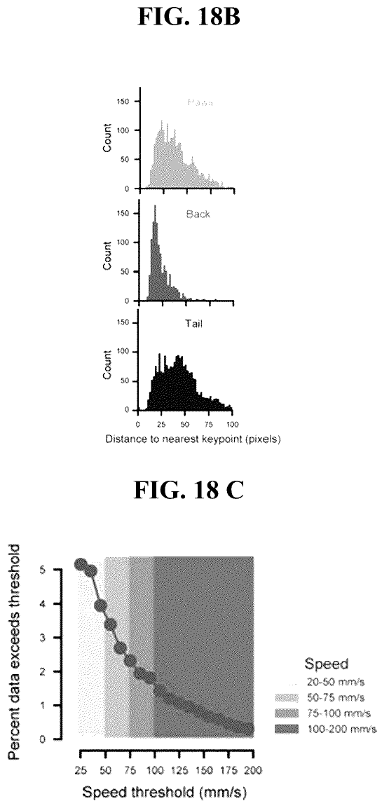

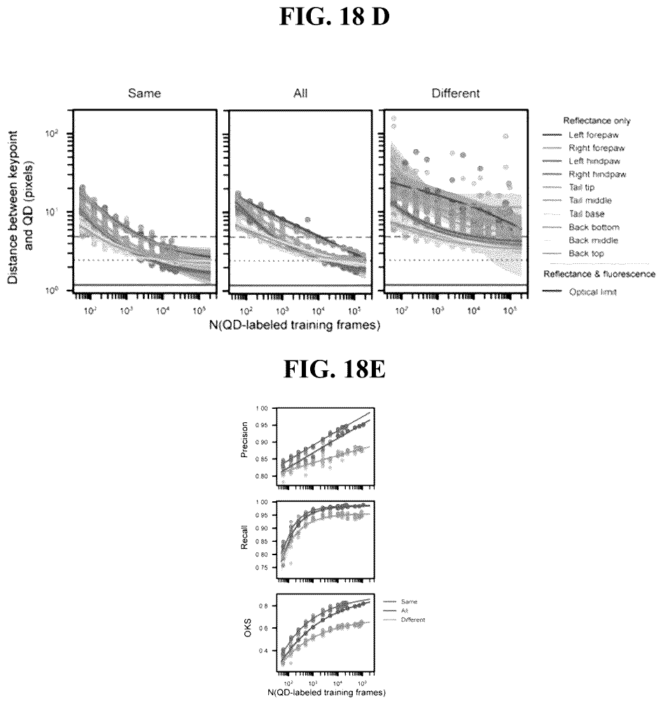

FIG. 18 describes details of QD-Pi-120K and models used in FIG. 4. FIG. 18A shows data for the “reflect+fluo” model from FIG. 4E, wherein the inventors determined the best set of parameters through a grid search of key U-Net parameters. Shown is the average held out pixel error for each parameter set. For the parameters not shown in each heatmap, the inventors computed the minimum error across all settings. FIG. 18B shows for the dataset used to train models in FIG. 4K (QD-Pi-120k), wherein the inventors computed, for each keypoint, the distance to the nearest neighbor. FIG. 18C shows that to determine the percent of QD-Pi-120k frames in which the mouse is moving at various speeds, the inventors calculated the velocity of the mouse's centroid using triangulated keypoints (see FIG. 17 and Methods for details) various thresholds between 25-200 mm/s and calculated the percent frames above each threshold. FIG. 18D shows the performance of the models shown in FIG. 4K broken down by body part for each condition. Conventions are the same as FIG. 4K. FIG. 18E shows different performance metrics for the models shown in FIG. 4K: precision, recall, and object keypoint similarity (OKS). Line reflects the median inverted power-law decay fit, and the shaded region indicates 95% bootstrap confidence interval.

FIG. 19 describes increasing the speed of version 2 of the imaging rig through reduced exposure times and increased analog gain. FIG. 19A is a schematic of the illumination sequence with shorter exposure times. Note that here fluorescence images are not background subtracted. FIG. 19B shows, Top, the time average of peak fluorescence intensity from n=5 mice injected with QD800.2 imaged at different exposure times and with different analog gains on each camera. Middle, the spread along the x-axis from mice imaged at different exposure times and gains measured using the standard deviation along the x-axis of the 2D autocorrelation function from the fluorescence channel. Bottom, the spread along the y-axis, computed using the same method as for the x-axis

### MATERIALS and METHODS

The inventors used quantum dot (QD) solutions that were suitable for in vivo use. For this manuscript, the inventors used four variants of quantum dots, all of which have a CdSe core and ZnS shell and are additionally coated with polyethylene glycol (PEG) for enhanced biocompatibility (QD800.2 is functionalized with polyarginine peptide instead of PEG).

### QD800 Variant 1 (QD800.1)

Due to their prior use in in vivo tracing of the vasculature in mice, the first variant the inventors tried was QTracker™ 800 Vascular Labels (ThermoFisher Scientific, #Q21071MP). These were used undiluted (2 μM).

### QD800 Variant 2 (QD800.2)

Due to their prior use in in vivo tracking inside of cells, the second variant the inventors tried was QTracker™ 800 Cell Labeling Kit (ThermoFisher Scientific, #Q25071MP). Qtracker® nanocrystals (Component A, 2 μM) was diluted by half with Qtracker® carrier (Component B).

### QD800 Variant 3 (QD800.3)

Both QD800.1 and QD800.2 were relatively short-lived in the skin. Larger particles, e.g., polymethyl-methacrylate (PMMA) beads, can persist in the skin for up to 5 years (46). Hence, the inventors hypothesized that attaching the quantum dots to larger biocompatible particles would enhance longevity. The inventors found using agarose beads to be ideal due to their porous structure and biocompatibility. Moreover, the inventors utilized the streptavidin-biotin reaction to immobilize the quantum dots onto the agarose beads.

High-capacity streptavidin agarose beads (Amid Biosciences, #SA-101-1) and custom-made biotin-conjugated quantum dots (ThermoFisher, per manufacturer's specification, ≥0.5 mg/mL) were used for agarose bead injections (see FIG. 11A for all microbead brand names that were tested). For suspension, sodium alginate (Sigma-Aldrich, #W201502-SAMPLE) was hydrolyzed in 1X phosphate-buffered saline (PBS) to make a 2% (w/v) solution. The beads were spun down, and the supernatant was removed. quantum dot solution was added at 2:1 (v: v) beads-to-quantum-dots ratio and gently mixed with the beads by pipetting up and down. The mixture was left to incubate at 40° C. for 1 hour and was mixed halfway through the incubation period to ensure saturation. After the incubation, the solution was mixed again and then washed three times with 1×PBS. The supernatant was removed, and the beads were resuspended in 2% sodium alginate in a 1:1 beads-to-alginate ratio to allow for an even distribution during injection.

### QD800 Variant 4 (QD800.4)

To enable biological targeting of quantum dots, the inventors used antibody-based targeting of QD800 to long-lived ECM-associated proteins, such as collagen and fibronectin. The exact concentration of quantum dots used in this kit was not disclosed by the manufacturer.

To conjugate QD800 to anti-Collagen I+III (Abcam, #ab34710) and anti-fibronectin (Abcam, #ab2413) rabbit polyclonal antibodies, the inventors capitalized on an enzyme- and click chemistry-mediated site-specific antibody conjugation approach (67) and used an antibody labeling kit (Invitrogen, SiteClick™ Antibody Labeling Kit, #70455). Anti-collagen I+III-QD800 and anti-fibronectin-QD800 antibody conjugates are referred to as QD800.4.COLLAGEN and QD800.4.FIBRONECTIN, respectively. Briefly, the antibody was concentrated using membrane filtration. Next, the carbohydrate domain of the antibody was modified for azide attachment using uridine-5′-diphosphate-N-Azidoacetylgalactosamine (UDP-GalNAz) labeling. Azide-modified antibody was then concentrated using membrane filtration and conjugated to DIBO-labeled QD800. To enhance the yield and concentration of conjugated antibodies (QD800.4) and to filter out unconjugated antibodies, the inventors purified QD800-conjugated antibodies using membrane filtration.

### Animals and Surgical Procedure

All procedures were approved by the Georgia Institute of Technology Institutional Animal Care and Use Committee (IACUC; protocol #A100557). Thirty-three male and seventeen female (7-20 weeks old) C57BL/6J mice were purchased from the Jackson Laboratories and kept under a reverse 12h light/12h dark cycle with ad libitum access to food and water. Specifically, for control, n=3 mice were not injected with any material (blank) and n=5 were injected with vehicle (vehicle, n=3 were used for Rig v1 as seen in FIG. 7 and n=2 for Rig v2. Blank and vehicle were combined in the control group). For QD800 injections, n=3 were injected with QD800.1, n=3 with QD800.2, n=6 with QD800.3 (n=3 were used for Rig v1 as seen in FIG. 7 and n=3 for Rig v2), and n=5 were injected with QD800.3 intra-articularly into the knee joints (n=3 were injected for in vivo recordings and n=2 cadavers were used for imaging). For quantum dot antibody injections, n=2 were injected with vehicle (described as n=2 for Rig v2 above), n=2 were injected with QD800.4.FIBRONECTIN, and another n=2 were injected with QD800.4.COLLAGEN. For immunofluorescence (IF) experiments, n=4 were injected with QD800.1 (tissues from n=2 were harvested at +30 minutes and n=2 were harvested at +4 hours post-injection), n=2 were injected with QD800.2 (tissues were harvested at +4 hours post-injection), and n-2 were injected with QD800.4.COLLAGEN (tissues were harvested at +8 days post-injection). For exposure time and volume experiments, n=9 were injected with QD800.2. For knee injection success rate experiments, n=4 were injected with QD800.3 intra-articularly into the knee joints. For FIG. 2B, n=1 mouse was injected with QD800.2 (sex was not determined for this animal). The animals were anesthetized with 4% isoflurane mixed in air using a nose cone. The procedures were carried out under 1.8-2% isoflurane anesthesia on a heating pad and completed within 30 minutes. The fur along the spine was shaved and further treated with a hair removal cream. The quantum dot mixtures were injected using a sterile pulled glass micro-pipette (Drummond 3-000-210-G) created using a Sutter P-2000 laser puller (parameters: heat 450, filament 4, velocity 150, delay 175, pull 35). The micro-pipette was attached to a modified positive-displacement micro-injector (Drummond 3-000-510-X). Specifically, the stainless-steel plunger of the micro-injector was cut to enable the pulled micropipette to be safely attached to the micro-injector. The tip of the micro-pipette was cut to the desired diameter for different QD800 injections (FIG. 11G). The quantum dot mixtures were delivered to each of the 14 injection sites subdermally: the paws (dorsal and ventral), the tail (base, midsection, and tip), and the back (upper, middle, and lower midline dorsal region). Following each injection, 70% ethanol and triple antibiotic ointment were applied to minimize potential infections. The animals were allowed to recover in their cage for one hour after the procedure.

A volume of 2 μL of QD800.1 and QD800.2 was delivered to the 14 injection sites subdermally with a 0.1-0.2 mm diameter micro-pipette. For agarose bead injections, the diameter of the micro-pipette was 0.3-0.4 mm to allow for the passage of the beads. The injection volume was changed to 4 μL to maximize the signal and was delivered to the same 14 injection sites subdermally.

An injection volume of 4 μL was also used to deliver QD800.4.FIBRONECTIN and QD800.4.COLLAGEN to the same 14 injection sites subdermally with a 0.1-0.2 mm diameter micro-pipette. The vehicle was repeated with the same injection volume alongside these experiments. Additionally, the fur along the spine was shaved and treated with a hair removal cream under anesthesia 4 and 6 weeks after QD800.4 injections to enhance the keypoint signal from the back of the mice.

The protocol for intra-articular knee injection was based on the protocol from Pitcher et al. paper and modified accordingly (49). The fur around the legs was shaved and treated with a hair removal cream. A volume of 4 μL of QD800.3 was delivered to each knee joint using a micro-pipette with a diameter of 0.25-0.3 mm to allow for bead passage and accurate access to the joint. For in vivo injections, post-operative analgesia was given at the start of the procedure, and following each injection, 70% ethanol and triple antibiotic ointment were applied. To confirm the targeting of the injections, the animals were placed under NIR illumination and imaged with a machine vision camera outfitted with a long-pass filter (see below for details on imaging hardware). The skin around the knee was tugged, and the signal was observed live to see if the fluorescence spot moved with the skin. Only the signal that remained stationary while moving the skin was deemed intra-articular. The animals were allowed to recover for one hour after the procedure. Here, n=3 mice were injected, and 2 out of 3 mice showed strong fluorescence in only one knee joint. The third mouse showed strong fluorescence in both knee joints and was thus used for analysis in FIG. 6E. In order to assess the success rate of knee injections, the inventors performed a second round of intra-articular injections into the knee joint. Here, n=4 animals were injected. Seven out of eight knee injections were successfully injected on the first try, while one knee was repeated due to pipette breaking.

For IF experiments, the back of the animals were shaved and treated with a hair removal cream. QD800.1, QD800.2, or QD800.4.COLLAGEN was delivered to multiple injection sites on each animal's back, following the same surgical procedure and respective parameters described above. The back skin tissues from the injection sites were harvested individually, where each tissue corresponds to one injection, at +4 hours for QD800.1 and QD800.2, at +30 minutes for QD800.1 control, and at +8 days for QD800.4.COLLAGEN post-injection. Extracted tissues for each condition were pooled and stored in individual tubes. For imaging and quantification, the inventors selected n=2 tissues for QD800.1 and n=2 tissues for QD800.2 to compare QD signal enrichment inside versus outside of cells; and n=1 tissue for QD800.1 as a control and n=1 tissue for QD800.4.COLLAGEN to compare their colocalization with collagen-I antibody.

### Recording Arena

A plexiglass arena was created from clear cast-acrylic panels (McMaster-Carr, #8560K184) to form an open-top cube with 29.845 cm edge length (i.e., same width, depth and height). Interdigitated patterns were cut into the edges of panels to ease fitting together. Panels were glued together using acrylic plastic cement (Sci-Grip, #10315). Custom 3D-printed molds were secured to the bottom acrylic panel (Torr-Seal Epoxy, Varian), and screw-to-expand brass inserts were inserted into the molds to secure 0.25-20-inches set screws. The other end of the set screws was inserted into Thorlabs 1-inch optical posts to, in turn, secure to an optical breadboard placed on top of a leveled frame (PFM52503).

### Recording Sessions

Mice were placed into the plexiglass chamber and imaged for approximately 3-5 minutes per session.

### Recording Hardware—Version 1

The plexiglass arena was filmed using five hardware-synchronized NIR-optimized Basler USB3 cameras (acA2040-90 μm). For wide field-of-view imaging, the cameras were outfitted with a Thorlabs machine vision lens with an 8 mm focal length (MVL8M1). In order to prevent imaging of QD excitation light, long-pass (MidOpt LP830-55) and polarization filters (PR1000-55) were secured to the lens. Polarization filters were rotated until excitation light was minimized. For exciting quantum dots at wavelengths compatible with imaging through skin, the inventors used NIR-I emitting LED lights (SL246-730IC) outfitted with polarization filters (PA371-142). For collecting reflectance images, IR-emitting LED lights were used (SL246-850IC). The lights were triggered in a temporally multiplexed configuration so that fluorescence and reflectance data could collected near-simultaneously using the following sequence: (1) IR lights on for 10 milliseconds (2) all lights off for 1 millisecond (3) NIR lights on for 23 milliseconds, (4) all lights off for 1.5 milliseconds. Power to the LED lights was supplied from a benchtop voltage source (B&K Precision BK1550) (FIG. 7B). To test if shorter exposure times could be used, a faster sequence was used in FIG. 19 only: IR lights on for 2 milliseconds, lights off for 1 millisecond, NIR lights on for 8 milliseconds, and all lights off for 6 milliseconds (here, a larger gap was used to allow for frame readout). Lights were triggered using 5V signals generated from an Arduino Uno, which was also used to trigger camera exposures via the GPIO lines on the Basler cameras. The illumination sequence was repeated for approximately 5 minutes for each recording session. The cameras were arranged in a pentagonal formation surrounding the plexiglass arena to capture mice at multiple angles.

### Recording Hardware—Version 2

The following modifications were made to the recording hardware version 1 (see above section) to maximize SNR. First, in order to enhance the excitation of the QD800 nanoparticles, the inventors decided to slightly blue-shift the excitation light (FIG. 4A). The 730 nm LED lights were replaced with 660 nm LED lights (Advanced Illumination SL-700150W-660). Due to the blue-shifted excitation light, polarizing filters were no longer needed to filter out stray light, so they were removed to enhance signal levels. Additionally, the long-pass filters were modified to accommodate the new wavelength, so they were replaced with 780 nm long-pass filters (MidOpt LP780-55).

### Recording Hardware—Optical Limit

The optical limit of the system, presented in FIGS. 4A, 4B, 4C, 4D, 4E, 4F, 4G, 4H, 4I, 4J, and 4K and FIGS. 6A, 6B, 6C, 6D, and 6E was estimated by calculating the Nyquist limit given a pixel size of 5.7 μm, an object distance of 304.8 mm (the approximate distance from each camera to the center of the plexiglass arena), and a lens focal length of 8 mm.

### Recording Hardware—Setup for Visualizing the Success of Knee Injections

The inventors constructed a compact setup under which the inventors could image QD fluorescence from intra-articular knee injections in anesthetized mice. LED lights (660 nm, Thorlabs M660L4 driven by a Thorlabs LEDD1B) outfitted with an adjustable lens (Thorlabs SM1U25-A) were used for exciting quantum dots. Fluorescence was imaged using an 830 nm long-pass filter (MidOpt LP830-28) mounted to the camera lens. Images were collected either using a Basler a2A3840-45ucBAS or acA2040-90 μm outfit with a 12 mm focal length lens (Thorlabs #MVL12M23) connected to a laptop running Basler pylon software. Non-fluorescence images were collected simply by removing the long-pass filter.

### Recording Software

Camera control and image acquisition software were written in Python. Custom software was also written for the Arduino to trigger the cameras over the appropriate GPIO line, along with the LED lights.

### Analysis of Quantum Dot Fluorescence

To assess the brightness and longevity of QD injections, the inventors computed a simple summary metric for each camera view and each session (FIG. 1F). First, to remove any contribution of the background, a rolling background (1500 frame sliding window, non-overlapping) was subtracted from the fluorescence frames (FIG. 8A). Next, to summarize the intensity on a per-frame basis, the inventors computed the maximum pixel value across x and y for every frame. Finally, to summarize either the average or peak intensity across time for each camera view and each recording session, the inventors computed either the mean or the 95th percentile across maximum frame intensities.

To confirm that full-frame calculations did not introduce artifacts into the analysis, the inventors repeated the calculation using segmentation masks and allowed us to ignore all pixels that did not belong to the mouse (FIGS. 8A, 8B, 8C, 8D, 8E, 8F, and 8G). In order to estimate the segmentation mask on reflectance frames, the inventors manually labeled 1,395 frames using the segments. ai platform. Then, the inventors used the nvidia/mit-b3 variant with default settings using pretrained weights from Huggingface.

Spatial autocorrelation was computed using the scipy.signal.fftconvolve function on background-subtracted fluorescence data. To minimize redundant calculations, the spatial autocorrelation was computed on every 200th frame. The autocorrelation on each frame was normalized such that the peak at the center was 1.

### Markerless Keypoint Tracking (Standard Keypoints, Tail, Back, Paws)

To track keypoints, the inventors utilized SLEAP (10). First, reflectance and fluorescence frames from all five camera views were alpha blended (90% fluorescence and 10% reflectance), uploaded to segments.ai, and were hand-labeled (n=862 total frames). Hand-labels were verified by a second labeler. Next, the hand-labeled, blended frames were used to train a single-instance U-Net to automatically identify body parts that coincided with fluorescence peaks. Eight hundred and sixty-two frames from all five cameras were hand-labeled using the segments.ai platform. the inventors performed a grid search over key hyperparameters (FIGS. 18A, 18B, 18C, and 18D), and used the following: filters 64, filters_rate 2.0 (this was set to 1.5 for sample efficiency experiments in FIG. 4K and FIG. 6E for computational efficiency since many models were fit), middle_block true, up_interpolate true, max_stride 64, stem_stride NULL, sigma (for head) 2.5, and output_stride 4.0. The final model had 502,521,418 parameters, and the model for sample efficiency experiments had 27,153,429 parameters. The following augmentation settings were used (any options not listed were set to false): rotate_min_angle-15, rotate_max_angle+15, translate_min-50, translate_max+50, scale_min 0.85, scale_max 1.15, gaussian_noise_mean 5.0, gaussian_noise_stddev 1.0, contrast_min gamma.5, contrast_max_gamma 2.0, random_flip true, random_flip_horizontal true. This network was then applied to the entire dataset. The predicted x/y position of each body part was then corrected using the fluorescence channel. In order to precisely localize each keypoint prediction, for each prediction, the inventors computed the fluorescence center within a 10-pixel radius surrounding the predicted keypoint.

To compute the center of fluorescence points, the inventors first used the OpenCV findContours function to identify a group of fluorescent points closest to the predicted keypoint. All pixels that did not belong to this group were set to 0. Next, a bivariate Gaussian was fit to the remaining fluorescence data. The center of the Gaussian was used as an estimate of the center of fluorescence (see FIGS. 15A, 15B, 15C, 15D, 15E, 15F, and 15G) for a comparison of methods for estimating the fluorescence center).

The fluorescence center of mass distance from reference point labels was compared with raw keypoint predictions in FIGS. 4G and 4H. the inventors refer to fluorescence localization as refinement. The center of mass was then used as the “reference point” x/y position for training and assessing the markerless keypoint trackers shown in FIG. 4K and FIG. 6E.

As a rule of thumb, given an approximately 10 mm span between many key landmarks on a mouse (18), the inventors assume that the average error of a given keypoint tracker should not exceed 2 mm, which is approximately 4.9 pixels given the optical setup (see Methods). A more stringent criterion of submillimeter error is likely required for accurately quantifying rodent kinematics (2.45 pixels on the setup), since submillimeter precision is common in human motion capture, and humans are approximately 20 times larger than mice (19-21). Here, the inventors define error as the average L2 distance between the network-identified keypoint and the corresponding QD fluorescence peak.

### Filtering Keypoint Predictions to Automatically Build New Training Datasets

To ensure that high-quality keypoints were used to build large training datasets for FIG. 4K and FIG. 6E, the inventors post-processed keypoint predictions using the following rules. First, the inventors assumed that any large jumps in position between frames indicated a tracking error, so keypoints were excluded if they moved more than 30 pixels (L2 distance) between neighboring frames. Next, to filter outliers, the inventors dimensionally reduced the x and y coordinates of the 10 keypoints (back bottom, back middle, back top, tail base, tail middle, tail tip, left/right fore/hindpaw) using principal components analysis (PCA). Here, the inventors selected the number of PCs required to drop the cumulative mean squared reconstruction error by 90%. Next, the inventors considered a keypoint visible if its keypoint confidence score exceeded 0.2. Of the visible keypoints, the inventors then set thresholds on the minimum amount of fluorescence and the maximum distance to the nearest QD center for the keypoint to be considered valid; both values scaled with keypoint confidence. Less confident keypoints required higher fluorescence and needed to be closer to the nearest QD center. The minimum fluorescence peak linearly scaled from 75 for predictions with a score of 0.7 to 25 for a score of 1.0. The maximum distance is scaled from 5 for predictions with a score of 0.7 to 15 for a score of 1.0. To prevent inclusion of frames with large difference between the number of “visible” keypoints and the number of “valid” keypoints, the inventors linearly scaled the number of accepted dropped keypoints (“visible”−“valid”) from 0 for frames with 3 or fewer keypoints to 3 for frames with 10 keypoints. Finally, to exclude frames with outliers, the inventors removed any frames where the distance between any pair of keypoints exceeded 300 pixels.

### Markerless Keypoint Tracking (Knee Joints)

For tracking of knee joints, all settings were the same as with standard keypoints, except for the following modifications. Eight hundred and seventy-two fused fluorescence and reflectance frames were manually labeled. Then, since the fluorescence data was extremely sparse (1 or 2 fluorescence spots per frame), the inventors trained a markerless keypoint tracker to identify keypoints using only the reflectance images. All post-processing thresholds were the same, except that the low confidence score used to linearly scale the fluorescence peak and distance to fluorescence center thresholds was set to 0.5 (rather than 0.7). Also, since there were only two keypoints when labeling the knee joints, the PCA post-processing filter was skipped.

Estimating Fluorescence Spread with Autocorrelation

To estimate the spread of fluorescence across time (FIG. 5F), the inventors took the spatial autocorrelation from every 200th frame (see above for more detail) and fit a bivariate Gaussian distribution using scipy.optimize.least_squares. The standard deviations along the x- and y-axes were used as estimates of spread.

### Human Keypoint Annotation and Analysis

Manual keypoint labeling was carried out independently by four labelers for reflectance data, and six labelers for overlaid reflectance and fluorescence data. Each labeler was presented with a series of images containing either overlaid reflectance and fluorescence camera frames (n=862 frames) or reflectance frames only (n=271 for the standard set of 10 keypoints shown in FIGS. 4A, 4B, 4C, 4D, 4E, 4F, 4G, 4H, 4I, 4J, and 4K, and a separate n=274 frames were labeled for knee joints shown in FIGS. 6A, 6B, 6C, 6D, and 6E) and asked to label keypoints visible on the camera frame. The frames were sampled randomly across all 5 cameras. Labelers all agreed on where keypoint locations should be, on average, prior to labeling, and individual frames were labeled independently. For fluorescence data, labelers marked the center of the fluorescent dot as the keypoint for the relevant body part. For reflectance data only, labelers estimated the location of keypoints based on the reflectance image used as a reference. If an area was not visible due to the current angle, the area was labeled as “hidden”. Labelers were blinded to the identity of the mice they were labeling.

Estimating Keypoint Performance Scaling with Number of Frames

To estimate how keypoint localization error (FIG. 4K, FIG. 6E, FIG. 18D) scaled with the size of the training dataset, the inventors fit a power-law decay function of the form,

y=a/(x+1)∧b+c

where x is the number of training frames and y is the keypoint localization error. The parameters a, b, and c were fit using the scipy.optimize.least_squares module. Then, to estimate how other performance characteristics (FIG. 18E) scaled with training dataset size the inventors used an inverted form of the same function,

y=a×(1−1/(x+1)∧b)+c

which was fit using the same method.

### Camera Calibration and Estimation of QD-Pi Error by Position in Arena

Intrinsics and extrinsics for all 5 cameras were estimated using a plexiglass cube with 4 separate ChArUco boards taped onto the outer faces—the top was left open to access the inside of the cube, and the bottom face was used for mounting onto the optical breadboard. The cube was spun by hand and frames were synchronously captured. Intrinsics and extrinsics were estimated using routines from OpenCV. Subsequent bundle adjustment of extrinsics was performed using the scipy.optimize.least_squares function.

In order to estimate the precision of QD-Pi as a function of position in the arena (FIGS. 17A and 17V), the inventors then used the keypoints predicted by the “reflect+fluo” model shown in FIGS. 4E, 4F, 4G and 4H. To produce an unbiased estimate of prediction error, no quality control thresholds were used (see above section, Filtering Keypoint Predictions to Automatically Build New Training Datasets). Then, to resolve keypoints in 3D, keypoints were triangulated using standard multi-view triangulation (68). Triangulated points and extrinsics were used to compute the distance of each keypoint relative to each camera view, and its angular distance from each camera's optical axis. the inventors use the distance between the predicted keypoint and the nearest QD fluorescence center to estimate the error in the prediction. The distance from the camera was used to convert the error from pixels to mms.

### Histology

For QD800 histology, tissues of interest at the injection sites (back skin, paws, and tail) were harvested and kept in 4% paraformaldehyde (PFA) solution for 48-72h at 4° C. before transferring them to 1X phosphate-buffered saline (PBS) solution. Prior to cryopreserving with sucrose, the paws and the tail were further dissected to collect the skin at the injection site. The tissues were placed in 15% and then 30% sucrose in 1×PBS until they sank. The tissues were embedded in optimal cutting temperature (OCT) compound (Sakura Finetek, #4583) and frozen with dry ice. They were then cryosectioned at 50 μm using a cryostat (ThermoFisher, CryoStar NX70), collected with microscopy slides, and stored at −80° C. until further processing. The sections were thawed, washed twice with 1×PBS, incubated in DAPI (1 μg/ml in water) (ThermoFisher, #D1306) for 3-5 minutes, and washed thrice with 1X PBS. The slides were then coverslipped using an anti-fade mounting medium (VectaShield, #H-1700).

The back skin tissues that were collected for IF assay and wheat germ agglutinin (WGA) staining went through the same tissue processing steps as described above. However, before freezing with dry ice, all samples except QD800.1- and QD800.2-injected samples went through vacuum infiltration. They were then cryosectioned at 20 μm, transferred onto frosted glass slides, and stored at −20° C. until further processing.

### Immunofluorescence and WGA Staining

To visualize the cell membrane, the inventors performed WGA staining. For this, the sections were thawed, washed thrice with 1×PBS, incubated in WGA conjugated to Alexa Fluor 488 (10 μg/ml in 1×PBS) (ThermoFisher, #W11261) for 1 hour, and washed 4 times with 1×PBS. The slides were then coverslipped using anti-fade mounting medium with DAPI (VectaShield, #H-1800).

For Collagen-I antibody IF staining, the inventors used a primary antibody derived from a species other than rabbit (host species of antibody used in QD800.4.COLLAGEN) or mouse to prevent antibody crosstalk and cross-reactivity. The sections were first thawed, washed thrice with 0.1 M phosphate buffer (PB). Then, the sections were blocked with 10% horse serum (ThermoFisher, #16050114) in 0.1M PB solution containing 0.3% Triton-X (PBTx) for 30 minutes at room temperature. Sections were then incubated in PBTx solution containing 1% horse serum and goat anti-type I Collagen antibody (1:500; SouthernBiotech, #1310-01) overnight at room temperature. Sections were then washed four times with 0.1M PB solution and further incubated in PBTx solution containing donkey anti-goat antibody conjugated to CF488A (Sigma-Aldrich, #SAB4600032) for 1.5 hours at room temperature. They were washed 4 times with 0.1M PB solution, and coverslipped using anti-fade mounting medium with DAPI (VectaShield, #H-1800).

### Imaging of Immunofluorescence and Staining

For QD800 histology, brightfield or fluorescence images were acquired at 4X and 10× magnification using a Nikon Eclipse Ti2 widefield epifluorescence microscope equipped with a penta-band excitation filter cube set (Semrock, LED-DA/FI/TR/Cy5/Cy7-A-000), Lumencor Spectra III light engine, and a Hamamatsu Orca Flash 3.0 CMOS sensor. The following excitation filters were used: DAPI (excitation wavelength or □ex=365 nm), CY7 (□ex=730 nm). Imaging parameters (e.g., exposure, laser power, etc.) for each excitation filter were kept consistent across all image acquisitions.

For IF and colocalization studies, images were acquired at 20X and 63× magnification using a laser scanning confocal microscope (ZEISS, LSM 900). The following excitation/emission filters were used: DAPI (2ex=353 nm, emission wavelength or λem=465 nm), AF488 (λex=493 nm, □em=517 nm), QD800 (λex=300 nm, λem=799 nm). Imaging parameters (e.g., laser power, binning, pixel dwell time, etc.) for each excitation/emission filter set were kept consistent across all image acquisitions.

### Quantification of Immunofluorescence and Staining

To estimate the tendency for QD800.2 to enter cells (FIGS. 2D and 2E), the ratio of QD fluorescence was calculated inside of cells to outside of cells. To achieve this, the inventors took the confocal images of DAPI, WGA, and QD fluorescence, and used the DAPI and WGA channels to estimate ROIs for each cell using Cellpose (69). Here, the flow threshold was set to 0.4, and the cellprob_threshold was set to 0.0. Then, the average QD fluorescence was computed per ROI along with the average QD fluorescence outside of all ROIs. The ratio between these two quantities, then provided an estimate of the tendency for quantum dots to enter cells.

Second, the inventors used confocal images of DAPI, collagen-I, and QD fluorescence (FIGS. 5B and 5C) to establish whether QD800.4.COLLAGEN co-localized with collagen. the inventors thresholded collagen-related fluorescence using Otsu's method to establish a collagen mask. DAPI fluorescence was also thresholded using the same technique. Then, the inventors calculated the ratio of QD fluorescence that co-localized with the collagen mask to QD fluorescence that co-localized with DAPI.

### IVIS Spectrum CT Imaging

Intra-articular knee injection of QD800.3 was performed into both knees of a mouse cadaver. The bottom half of the mouse was shaved and further treated with hair removal cream to reduce imaging artifacts.

IVIS Spectrum CT Imaging system (Perkin Elmer/Revvity) was used to image the right knee in Fluorescence Tomography (FLIT) mode using a set of trans-illumination points combined with CT-based reconstruction of the skeletal structure. Living Image 4.7.4 software was used to reconstruct the fluorescence light source (QD800) based on this trans-illumination at selected locations. The reconstructed fluorescence was then overlaid with both surface reconstruction of the animal and its skeletal structure to create a 3D representation of the injected QD800.3. Only results from the right knee are shown since the left knee imaging parameters were not appropriately optimized for this demonstration.

### Statistics

All hypothesis tests were two-tailed and non-parametric. Where appropriate, the inventors list the exact p-value, sample size (along with definition of a sample), and the exact value of the relevant test statistic. Results are expressed as mean±standard deviation. In all figures, * denotes p<0.05, ** p<0.01, and *** p<0.001. Effect sizes for Mann-Whitney U tests are given as common language effect sizes, f. Effect sizes for Wilcoxon signed-rank tests are given as the rank-biserial correlation, r. Boxplots conventions are as follows. Boxes extend from the first quartile to the third quartile of the data, with a line specifying the median. The whiskers extend to the farthest datapoint within 1.5-fold of the interquartile range of the data. Outliers beyond those points were not plotted.

### REFERENCES

Ash, C., Dubec, M., Donne, K., & Bashford, T. (2017). Effect of wavelength and beam width on penetration in light-tissue interaction using computational methods. Lasers Med Sci, 32 (8), 1909-1918.
Aswathy, R. G., Yoshida, Y., Maekawa, T., & Kumar, D. S. (2010). Near-infrared quantum dots for deep tissue imaging. Analytical and Bioanalytical Chemistry, 397 (4), 1417-1435.
Ballou, B., Ernst, L. A., Andreko, S., Fitzpatrick, J. A., Lagerholm, B. C., Waggoner, A. S., & Bruchez, M. P. (2009). Imaging vasculature and lymphatic flow in mice using quantum dots. Methods Mol Biol, 574, 63-74.
Ballou, B., Lagerholm, B. C., Ernst, L. A., Bruchez, M. P., & Waggoner, A. S. (2004). Noninvasive Imaging of Quantum Dots in Mice. Bioconjugate Chemistry, 15 (1), 79-86.
Bauman, J. M., & Chang, Y.-H. (2010). High-speed X-ray video demonstrates significant skin movement errors with standard optical kinematics during rat locomotion. Journal of Neuroscience Methods, 186 (1), 18-24.
Berman, G. J., Choi, D. M., Bialek, W., & Shaevitz, J. W. (2014). Mapping the stereotyped behaviour of freely moving fruit flies. Journal of The Royal Society Interface, 11 (99), 20140672.
Berridge, K. C., & Fentress, J. C. (1987). Disruption of natural grooming chains after striatopallidal lesions. Psychobiology, 15 (4), 336-342.
Biderman, D., Whiteway, M. R., Hurwitz, C., Greenspan, N., Lee, R. S., Vishnubhotla, A., Warren, R., Pedraja, F., Noone, D., Schartner, M., Huntenburg, J. M., Khanal, A., Meijer, G. T., Noel, J.-P., Pan-Vazquez, A., Socha, K. Z., Urai, A. E., Laboratory, T. I. B., Cunningham, J. P., . . . . Paninski, L. (2023). Lightning Pose: improved animal pose estimation via semi-supervised learning, Bayesian ensembling, and cloud-native open-source tools. bioRxiv, 2023.2004.2028.538703.
Blanco, E., Shen, H., & Ferrari, M. (2015). Principles of nanoparticle design for overcoming biological barriers to drug delivery. Nature Biotechnology, 33 (9), 941-951.
Bonnan, M. F., Shulman, J., Varadharajan, R., Gilbert, C., Wilkes, M., Horner, A., & Brainerd, E. (2016). Forelimb Kinematics of Rats Using XROMM, with Implications for Small Eutherians and Their Fossil Relatives. PLOS ONE, 11 (3), e0149377.
Brainerd, E. L., Baier, D. B., Gatesy, S. M., Hedrick, T. L., Metzger, K. A., Gilbert, S. L., & Crisco, J. J. (2010). X-ray reconstruction of moving morphology (XROMM): precision, accuracy and applications in comparative biomechanics research. J Exp Zool A Ecol Genet Physiol, 313 (5), 262-279.
Butler, D. J., Keim, A. P., Ray, S., & Azim, E. (2022). Large-scale capture of hidden fluorescent labels for training generalizable markerless motion capture models. bioRxiv, 2022.2007.2007.499213.
Dunn, T. W., Marshall, J. D., Severson, K. S., Aldarondo, D. E., Hildebrand, D. G. C., Chettih, S. N., Wang, W. L., Gellis, A. J., Carlson, D. E., Aronov, D., Freiwald, W. A., Wang, F., & Ölveczky, B. P. (2021). Geometric deep learning enables 3D kinematic profiling across species and environments. Nat Methods, 18 (5), 564-573.
Fentress, J. C., & Stilwell, F. P. (1973). Grammar of a Movement Sequence in Inbred Mice. Nature, 244 (5410), 52-53.
Filipe, V. M., Pereira, J. E., Costa, L. M., Maurício, A. C., Couto, P. A., Melo-Pinto, P., & Varejão, A. S. P. (2006). Effect of skin movement on the analysis of hindlimb kinematics during treadmill locomotion in rats. Journal of Neuroscience Methods, 153 (1), 55-61. Gil, H. M., Price, T. W., Chelani, K., Bouillard, J.-S. G., Calaminus, S. D. J., & Stasiuk,
G. J. (2021). NIR-quantum dots in biomedical imaging and their future. iScience, 24 (3), 102189.
Gopee, N. V., Roberts, D. W., Webb, P., Cozart, C. R., Siitonen, P. H., Warbritton, A. R., Yu, W. W., Colvin, V. L., Walker, N. J., & Howard, P. C. (2007). Migration of intradermally injected quantum dots to sentinel organs in mice. Toxicol Sci, 98 (1), 249-257.
Graving, J. M., Chae, D., Naik, H., Li, L., Koger, B., Costelloe, B. R., & Couzin, I. D. (2019). DeepPoseKit, a software toolkit for fast and robust animal pose estimation using deep learning. eLife, 8, e47994.
Hsu, A. I., & Yttri, E. A. (2021). B-SOiD, an open-source unsupervised algorithm for identification and fast prediction of behaviors. Nat Commun, 12 (1), 5188.
Ignatowska-Jankowska, B. M., Ozer, A. G., Kuck, A., Niphakis, M. J., Ogasawara, D., Cravatt, B. F., & Uusisaari, M. Y. (2023). Stimulatory effect of monoacylglycerol lipase inhibitor MJN110 on locomotion and step kinematics demonstrated by high-precision 3D motion capture in mice. bioRxiv, 2023.2006.2025.546437.
Karashchuk, P., Rupp, K. L., Dickinson, E. S., Walling-Bell, S., Sanders, E., Azim, E., Brunton, B. W., & Tuthill, J. C. (2021). Anipose: A toolkit for robust markerless 3D pose estimation. Cell Reports, 36 (13), 109730.
Kobayashi, H., Hama, Y., Koyama, Y., Barrett, T., Regino, C. A. S., Urano, Y., & Choyke, P. L. (2007). Simultaneous Multicolor Imaging of Five Different Lymphatic Basins Using Quantum Dots. Nano Letters, 7 (6), 1711-1716.
Lemperle, G., Hazan-Gauthier, N., & Lemperle, M. (1995). PMMA microspheres (Artecoll) for skin and soft-tissue augmentation. Part II: Clinical investigations. Plast Reconstr Surg, 96 (3), 627-634.
Marshall, J. D., Aldarondo, D. E., Dunn, T. W., Wang, W. L., Berman, G. J., & Ölveczky, B. P. (2021). Continuous Whole-Body 3D Kinematic Recordings across the Rodent Behavioral Repertoire. Neuron, 109 (3), 420-437.e428.
Mathis, A., Mamidanna, P., Cury, K. M., Abe, T., Murthy, V. N., Mathis, M. W., & Bethge, M. (2018). DeepLabCut: markerless pose estimation of user-defined body parts with deep learning. Nature Neuroscience, 21 (9), 1281-1289.
Medintz, I. L., Uyeda, H. T., Goldman, E. R., & Mattoussi, H. (2005). Quantum dot bioconjugates for imaging, labelling and sensing. Nature Materials, 4 (6), 435-446.
Monsees, A., Voit, K.-M., Wallace, D. J., Sawinski, J., Charyasz, E., Scheffler, K., Macke, J. H., & Kerr, J. N. D. (2022). Estimation of skeletal kinematics in freely moving rodents. Nature Methods, 19 (11), 1500-1509.
Peirson, S. N., Brown, L. A., Pothecary, C. A., Benson, L. A., & Fisk, A. S. (2018). Light and the laboratory mouse. J Neurosci Methods, 300, 26-36.
Pereira, T. D., Tabris, N., Matsliah, A., Turner, D. M., Li, J., Ravindranath, S., Papadoyannis, E. S., Normand, E., Deutsch, D. S., Wang, Z. Y., Mckenzie-Smith, G. C., Mitelut, C. C., Castro, M. D., D'Uva, J., Kislin, M., Sanes, D. H., Kocher, S. D., Wang, S. S. H., Falkner, A. L., . . . Murthy, M. (2022). SLEAP: A deep learning system for multi-animal pose tracking. Nature Methods, 19 (4), 486-495.
Pi, Q. M., Zhang, W. J., Zhou, G. D., Liu, W., & Cao, Y. (2010). Degradation or excretion of quantum dots in mouse embryonic stem cells. BMC Biotechnol, 10, 36.
Rak-Raszewska, A., Marcello, M., Kenny, S., Edgar, D., Sée, V., & Murray, P. (2012). Quantum dots do not affect the behaviour of mouse embryonic stem cells and kidney stem cells and are suitable for short-term tracking. PLOS ONE, 7 (3), e32650.
Ramalingasetty, S. T., Danner, S. M., Arreguit, J., Markin, S. N., Rodarie, D., Kathe, C., Courtine, G., Rybak, I. A., & Ijspeert, A. J. (2021). A Whole-Body Musculoskeletal Model of the Mouse. IEEE Access, 9, 163861-163881.
Reineck, P., & Gibson, B. C. (2017). Near-Infrared Fluorescent Nanomaterials for Bioimaging and Sensing. Advanced Optical Materials, 5 (2), 1600446.
Resch-Genger, U., Grabolle, M., Cavaliere-Jaricot, S., Nitschke, R., & Nann, T. (2008). Quantum dots versus organic dyes as fluorescent labels. Nature Methods, 5 (9), 763-775.
Sabino, C. P., Deana, A. M., Yoshimura, T. M., da Silva, D. F., França, C. M., Hamblin, M. R., & Ribeiro, M. S. (2016). The optical properties of mouse skin in the visible and near infrared spectral regions. J Photochem Photobiol B, 160, 72-78.
Soo Choi, H., Liu, W., Misra, P., Tanaka, E., Zimmer, J. P., Itty Ipe, B., Bawendi, M. G., & Frangioni, J. V. (2007). Renal clearance of quantum dots. Nature Biotechnology, 25 (10), 1165-1170.
Wagner, A. M., Knipe, J. M., Orive, G., & Peppas, N. A. (2019). Quantum dots in biomedical applications. Acta Biomater, 94, 44-63.
Weinreb, C., Pearl, J., Lin, S., Osman, M. A. M., Zhang, L., Annapragada, S., Conlin, E., Hoffman, R., Makowska, S., Gillis, W. F., Jay, M., Ye, S., Mathis, A., Mathis, M. W., Pereira, T., Linderman, S. W., & Datta, S. R. (2023). Keypoint-MoSeq: parsing behavior by linking point tracking to pose dynamics. bioRxiv.
Witte, H., Biltzinger, J., Hackert, R. m., Schilling, N., Schmidt, M., Reich, C., & Fischer, M. S. (2002). Torque patterns of the limbs of small therian mammals during locomotion on flat ground. Journal of Experimental Biology, 205 (9), 1339-1353.
Zhang, M., Yue, J., Cui, R., Ma, Z., Wan, H., Wang, F., Zhu, S., Zhou, Y., Kuang, Y.,
Zhong, Y., Pang, D.-W., & Dai, H. (2018). Bright quantum dots emitting at ˜1,600 nm in the NIR-IIb window for deep tissue fluorescence imaging. Proceedings of the National Academy of Sciences, 115 (26), 6590-6595.
Lemperle, G., Hazan-Gauthier, N., & Lemperle, M. (1995). PMMA microspheres (Artecoll) for skin and soft-tissue augmentation. Part II: Clinical investigations. Plast Reconstr Surg, 96 (3), 627-634. Pereira, T. D., Tabris, N., Matsliah, A., Turner, D. M., Li, J., Ravindranath, S.,
Papadoyannis, E. S., Normand, E., Deutsch, D. S., Wang, Z. Y., Mckenzie-Smith, G. C., Mitelut, C. C., Castro, M. D., D'Uva, J., Kislin, M., Sanes, D. H., Kocher, S. D., Wang, S. S. H., Falkner, A. L., . . . . Murthy, M. (2022). SLEAP: A deep learning system for multi-animal pose tracking. Nature Methods, 19 (4), 486-495.
J. C. Fentress, F. P. Stilwell, Grammar of a Movement Sequence in Inbred Mice. Nature 244, 52-53 (1973).
2. I. Q. Whishaw, S. M. Pellis, The structure of skilled forelimb reaching in the rat: A proximally driven movement with a single distal rotatory component. Behavioural Brain Research 41, 49-59 (1990).
3. I. Q. Whishaw, W. T. O'Connor, S. B. Dunnett, THE CONTRIBUTIONS OF MOTOR CORTEX, NIGROSTRIATAL DOPAMINE AND CAUDATE-PUTAMEN TO SKILLED FORELIMB USE IN THE RAT. Brain 109, 805-843 (1986).
4. K. C. Berridge, J. C. Fentress, Disruption of natural grooming chains after striatopallidal lesions. Psychobiology 15, 336-342 (1987).
5. D. Biderman, M. R. Whiteway, C. Hurwitz, N. Greenspan, R. S. Lee, A. Vishnubhotla, R. Warren, F. Pedraja, D. Noone, M. M. Schartner, J. M. Huntenburg, A. Khanal, G. T. Meijer, J.-P. Noel, A. Pan-Vazquez, K. Z. Socha, A. E. Urai, L. Abbot, L. Acerbi, V. Aguillon-Rodriguez, M. Ahmadi, J. Amjad, D. Angelaki, J. Arlandis, Z. C. Ashwood, K. Banga, H. Barrell, H. M. Bayer, B. Benson, J. Benson, J. Bhagat, D. Birman, N. Bonacchi, K. Bougrova, J. Boussard, S. A. Bruijns, E. K. Buchanan, R. Campbell, M. Carandini, J. A. Catarino, F. Cazettes, G. A. Chapuis, A. K. Churchland, Y. Dan, F. Davatolhagh, P. Dayan, S. Denève, E. E. J. DeWitt, L. L. Dong, T. Engel, M. Fabbri, M. Faulkner, R. Fetcho, I. Fiete, C. Findling, L. Freitas-Silva, S. Ganguli, B. Gercek, N. Ghani, I. Gordeliy, L. M. Haetzel, K. D. Harris, M. Hausser, N. Hiratani, S. Hofer, F. Hu, F. Huber, C. Hurwitz, A. Khanal, C. S. Krasniak, S. Krishnagopal, M. Krumin, D. Kundu, A. Landemard, C. Langdon, C. Langfield, I. Laranjeira, P. Latham, P. Lau, H. D. Lee, A. Liu, Z. F. Mainen, A. Makri-Cottington, H. Martinez-Vergara, B. McMannon, I. McRoberts, G. T. Meijer, M. Melin, L. Meshulam, K. Miller, N. J. Miska, C. Mitelut, Z. Mohammadi, T. Mrsic-Flogel, M. Murakami, J.-P. Noel, K. Nylund, F. Oloomi, A. Pan-Vazquez, L. Paninski, A. Pezzotta, S. Picard, J. W. Pillow, A. Pouget, F. Rau, C. Rossant, N. Roth, N. A. Roy, K. Saniee, R. Schaeffer, M. M. Schartner, Y. Shi, C. Soares, K. Z. Socha, C. Soitu, N. A. Steinmetz, K. Svoboda, M. Taheri, C. Tessereau, A. E. Urai, E. Varol, M. J. Wells, S. J. West, M. R. Whiteway, C. Windolf, O. Winter, I. Witten, L. E. Wool, Z. Xu, H. Yu, A. M. Zador, Y. Zhang, J. P. Cunningham, N. B. Sawtell, L. Paninski, T. I. B. Laboratory, Lightning Pose: improved animal pose estimation via semi-supervised learning, Bayesian ensembling and cloud-native open-source tools. Nat Methods 21, 1316-1328 (2024).
6. T. W. Dunn, J. D. Marshall, K. S. Severson, D. E. Aldarondo, D. G. C. Hildebrand, S. N. Chettih, W. L. Wang, A. J. Gellis, D. E. Carlson, D. Aronov, W. A. Freiwald, F. Wang, B. P. Ölveczky, Geometric deep learning enables 3D kinematic profiling across species and environments. Nat Methods 18, 564-573 (2021).
7. J. M. Graving, D. Chae, H. Naik, L. Li, B. Koger, B. R. Costelloe, I. D. Couzin, DeepPoseKit, a software toolkit for fast and robust animal pose estimation using deep learning. Elife 8, e47994 (2019).
8. P. Karashchuk, K. L. Rupp, E. S. Dickinson, S. Walling-Bell, E. Sanders, E. Azim, B. W. Brunton, J. C. Tuthill, Anipose: A toolkit for robust markerless 3D pose estimation. Cell Rep 36, 109730 (2021).
9. A. Mathis, P. Mamidanna, K. M. Cury, T. Abe, V. N. Murthy, M. W. Mathis, M. Bethge, DeepLabCut: markerless pose estimation of user-defined body parts with deep learning. Nat Neurosci 21, 1281-1289 (2018).
10. T. D. Pereira, N. Tabris, A. Matsliah, D. M. Turner, J. Li, S. Ravindranath, E. S. Papadoyannis, E. Normand, D. S. Deutsch, Z. Y. Wang, G. C. Mckenzie-Smith, C. C. Mitelut, M. D. Castro, J. D′Uva, M. Kislin, D. H. Sanes, S. D. Kocher, S. S. H. Wang, A. L. Falkner, J. W. Shaevitz, M. Murthy, SLEAP: A deep learning system for multi-animal pose tracking. Nat Methods 19, 486-495 (2022).
11. O. Ronneberger, P. Fischer, T. Brox, U-Net: Convolutional Networks for Biomedical Image Segmentation. Lecture Notes in Computer Science (including subseries Lecture Notes in Artificial Intelligence and Lecture Notes in Bioinformatics) 9351, 234-241 (2015).
12. G. J. Berman, D. M. Choi, W. Bialek, J. W. Shaevitz, Mapping the stereotyped behaviour of freely moving fruit flies. J R Soc Interface 11, 20140672 (2014).
13. A. I. Hsu, E. A. Yttri, B-SOiD, an open-source unsupervised algorithm for identification and fast prediction of behaviors. Nat Commun 12, 5188 (2021).
14. C. Weinreb, J. E. Pearl, S. Lin, M. A. M. Osman, L. Zhang, S. Annapragada, E. Conlin, R. Hoffmann, S. Makowska, W. F. Gillis, M. Jay, S. Ye, A. Mathis, M. W. Mathis, T. Pereira, S. W. Linderman, S. R. Datta, Keypoint-MoSeq: parsing behavior by linking point tracking to pose dynamics. Nat Methods 21, 1329-1339 (2024).
15. M. I. Becker, D. J. Calame, J. Wrobel, A. L. Person, Online control of reach accuracy in mice. J Neurophysiol 124, 1637-1655 (2020).
16. K. Huang, Q. Yang, Y. Han, Y. Zhang, Z. Wang, L. Wang, P. Wei, An Easily Compatible Eye-tracking System for Freely-moving Small Animals. Neurosci Bull 38, 661-676 (2022).
17. J. D. Marshall, D. E. Aldarondo, T. W. Dunn, W. L. Wang, G. J. Berman, B. P. Ölveczky, Continuous Whole-Body 3D Kinematic Recordings across the Rodent Behavioral Repertoire. Neuron 109, 420-437.e8 (2021).
18. S. T. Ramalingasetty, S. M. Danner, J. Arreguit, S. N. Markin, D. Rodarie, C. Kathe, G. Courtine, I. A. Rybak, A. J. Ijspeert, A Whole-Body Musculoskeletal Model of the Mouse. IEEE Access 9, 163861-163881 (2021).
19. S. Das, L. Trutoiu, A. Murai, D. Alcindor, M. Oh, F. D. la Torre, J. Hodgins, “Quantitative measurement of motor symptoms in Parkinson's disease: A study with full-body motion capture data” in 2011 Annual International Conference of the IEEE Engineering in Medicine and Biology Society (2011), pp. 6789-6792.
20. B. Kadirvelu, C. Gavriel, S. Nageshwaran, J. P. K. Chan, S. Nethisinghe, S. Athanasopoulos, V. Ricotti, T. Voit, P. Giunti, R. Festenstein, A. A. Faisal, A wearable motion capture suit and machine learning predict disease progression in Friedreich's ataxia. Nat Med 29, 86-94 (2023).
21. V. Ricotti, B. Kadirvelu, V. Selby, R. Festenstein, E. Mercuri, T. Voit, A. A. Faisal, Wearable full-body motion tracking of activities of daily living predicts disease trajectory in Duchenne muscular dystrophy. Nat Med 29, 95-103 (2023).
22. J. M. Bauman, Y.-H. Chang, High-speed X-ray video demonstrates significant skin movement errors with standard optical kinematics during rat locomotion. J Neurosci Methods 186, 18-24 (2010).
23. V. M. Filipe, J. E. Pereira, L. M. Costa, A. C. Maurício, P. A. Couto, P. Melo-Pinto, A. S. P. Varejão, Effect of skin movement on the analysis of hindlimb kinematics during treadmill locomotion in rats. J Neurosci Methods 153, 55-61 (2006).
24. A. Leardini, L. Chiari, U. Della Croce, A. Cappozzo, Human movement analysis using stereophotogrammetry: Part 3. Soft tissue artifact assessment and compensation. Gait Posture 21, 212-225 (2005).
25. R. R. Neptune, M. L. Hull, Accuracy assessment of methods for determining hip movement in seated cycling. J Biomech 28, 423-437 (1995).
26. A. Monsees, K.-M. Voit, D. J. Wallace, J. Sawinski, E. Charyasz, K. Scheffler, J. H. Macke, J. N. D. Kerr, Estimation of skeletal kinematics in freely moving rodents. Nat Methods 19, 1500-1509 (2022).
27. M. A. Lafortune, P. R. Cavanagh, H. J. Sommer, A. Kalenak, Three-dimensional kinematics of the human knee during walking. J Biomech 25, 347-357 (1992).
28. J. L. Lanovaz, S. Khumsap, H. M. Clayton, Quantification of three-dimensional skin displacement artefacts on the equine tibia and third metatarsus. Equine and Comparative Exercise Physiology 1, 141-150 (2004).
29. S. Tashman, W. Anderst, In-Vivo Measurement of Dynamic Joint Motion Using High Speed Biplane Radiography and CT: Application to Canine ACL Deficiency. J Biomech Eng 125, 238-245 (2003).
30. M. F. Bonnan, J. Shulman, R. Varadharajan, C. Gilbert, M. Wilkes, A. Horner, E. Brainerd, Forelimb Kinematics of Rats Using XROMM, with Implications for Small Eutherians and Their Fossil Relatives. PLOS One 11, e0149377-(2016).
31. E. L. Brainerd, D. B. Baier, S. M. Gatesy, T. L. Hedrick, K. A. Metzger, S. L. Gilbert, J. J. Crisco, X-ray reconstruction of moving morphology (XROMM): precision, accuracy and applications in comparative biomechanics research. J Exp Zool A Ecol Genet Physiol 313A, 262-279 (2010).
32. H. Witte, J. Biltzinger, R. Hackert, N. Schilling, M. Schmidt, C. Reich, M. S. Fischer, Torque patterns of the limbs of small therian mammals during locomotion on flat ground. Journal of Experimental Biology 205, 1339-1353 (2002).
33. C. Ash, M. Dubec, K. Donne, T. Bashford, Effect of wavelength and beam width on penetration in light-tissue interaction using computational methods. Lasers Med Sci 32, 1909-1918 (2017).
34. P. Reineck, B. C. Gibson, Near-Infrared Fluorescent Nanomaterials for Bioimaging and Sensing. Adv Opt Mater 5, 1600446 (2017).
35. C. P. Sabino, A. M. Deana, T. M. Yoshimura, D. F. T. da Silva, C. M. França, M. R. Hamblin, M. S. Ribeiro, The optical properties of mouse skin in the visible and near infrared spectral regions. J Photochem Photobiol B 160, 72-78 (2016).
36. K. Licha, “Contrast Agents for Optical Imaging” in Contrast Agents II: Optical, Ultrasound, X-Ray and Radiopharmaceutical Imaging, W. Krause, Ed. (Springer Berlin Heidelberg, Berlin, Heidelberg, 2002; https://doi.org/10.1007/3-540-46009-8_1), pp. 1-29.
37. D. Baker, C. Xu, Y. Liu, B. Arús, K. Mishra, M. Luciano, V. Bandi, A. Kumar, Z. Guo, M. Bick, M. Xu, K. Zhang, J. Lingg, J. Bae, A. Kang, S. Gerben, A. Bera, J. Vaughan, J. Manton, Y. Guan, De Novo Design of Near Infrared Fluorescent Proteins (2024).
38. U. Resch-Genger, M. Grabolle, S. Cavaliere-Jaricot, R. Nitschke, T. Nann, Quantum dots versus organic dyes as fluorescent labels. Nat Methods 5, 763-775 (2008).
39. A. M. Wagner, J. M. Knipe, G. Orive, N. A. Peppas, Quantum dots in biomedical applications. Acta Biomater 94, 44-63 (2019).
40. R. G. Aswathy, Y. Yoshida, T. Maekawa, D. S. Kumar, Near-infrared quantum dots for deep tissue imaging. Anal Bioanal Chem 397, 1417-1435 (2010).
41. E. Blanco, H. Shen, M. Ferrari, Principles of nanoparticle design for overcoming biological barriers to drug delivery. Nat Biotechnol 33, 941-951 (2015).
42. H. M. Gil, T. W. Price, K. Chelani, J.-S. G. Bouillard, S. D. J. Calaminus, G. J. Stasiuk, NIR-quantum dots in biomedical imaging and their future. iScience 24, 102189 (2021).
43. M. Zhang, J. Yue, R. Cui, Z. Ma, H. Wan, F. Wang, S. Zhu, Y. Zhou, Y. Kuang, Y. Zhong, D.-W. Pang, H. Dai, Bright quantum dots emitting at ˜1,600 nm in the NIR-IIb window for deep tissue fluorescence imaging. Proceedings of the National Academy of Sciences 115, 6590-6595 (2018).
44. B. Ballou, L. A. Ernst, S. Andreko, J. A. Fitzpatrick, B. C. Lagerholm, A. S. Waggoner, M. P. Bruchez, Imaging vasculature and lymphatic flow in mice using quantum dots. Methods Mol Biol 574, 63-74 (2009).
45. B. Ballou, B. C. Lagerholm, L. A. Ernst, M. P. Bruchez, A. S. Waggoner, Noninvasive Imaging of Quantum Dots in Mice. Bioconjug Chem 15, 79-86 (2004).
46. G. Lemperle, N. Hazan-Gauthier, M. Lemperle, PMMA Microspheres (Artecoll) for Skin and Soft-Tissue Augmentation. Part II: Clinical Investigations. Plast Reconstr Surg 96 (1995).
47. W. Dauer, S. Przedborski, Parkinson's Disease: Mechanisms and Models. Neuron 39, 889-909 (2003).
48. S. N. Peirson, L. A. Brown, C. A. Pothecary, L. A. Benson, A. S. Fisk, Light and the laboratory mouse. J Neurosci Methods 300, 26-36 (2018).
49. T. Pitcher, J. Sousa-Valente, M. Malcangio, The Monoiodoacetate Model of Osteoarthritis Pain in the Mouse. JoVE, e53746 (2016).
50. D. J. Butler, A. P. Keim, S. Ray, E. Azim, Large-scale capture of hidden fluorescent labels for training generalizable markerless motion capture models. Nat Commun 14, 5866 (2023).
51. B. M. Ignatowska-Jankowska, L. I. Swaminathan, T. H. Turkki, D. Sakharuk, A. G. Ozer, A. Kuck, M. Y. Uusisaari, Accurate tracking of locomotory kinematics in mice moving freely in three-dimensional environments. eNeuro, ENEURO.0045-25.2025 (2025).
52. G. Moukarzel, B. C. Rauscher, C. A. Patil, A. J. Spence, Infrared videography of a subcutaneous knee tattoo as a simple and inexpensive method to overcome skin motion artifact in rodent kinematics. bioRxiv, 2022.08.22.504813 (2022).
53. Q. M. Pi, W. J. Zhang, G. D. Zhou, W. Liu, Y. Cao, Degradation or excretion of quantum dots in mouse embryonic stem cells. BMC Biotechnol 10, 36 (2010).
54. A. Rak-Raszewska, M. Marcello, S. Kenny, D. Edgar, V. Sée, P. Murray, Quantum Dots Do Not Affect the Behaviour of Mouse Embryonic Stem Cells and Kidney Stem Cells and Are Suitable for Short-Term Tracking. PLOS One 7, e32650-(2012).
55. J. L. Kovar, X. Xu, D. Draney, A. Cupp, M. A. Simpson, D. Michael Olive, Near-infrared-labeled tetracycline derivative is an effective marker of bone deposition in mice. Anal Biochem 416, 167-173 (2011).
56. S. Jia, E. Y. Lin, E. B. Mobley, I. Lim, L. Guo, S. Kallepu, P. S. Low, E. M. Sletten, Water-soluble chromenylium dyes for shortwave infrared imaging in mice. Chem 9, 3648-3665 (2023).
57. B. A. Arus, E. D. Cosco, J. Yiu, I. Balba, T. S. Bischof, E. M. Sletten, O. T. Bruns, Shortwave infrared fluorescence imaging of peripheral organs in awake and freely moving mice. Front Neurosci 17, 1135494 (2023).
58. E. D. Cosco, B. A. Arus, A. L. Spearman, T. L. Atallah, I. Lim, O. S. Leland, J. R. Caram, T. S. Bischof, O. T. Bruns, E. M. Sletten, Bright Chromenylium Polymethine Dyes Enable Fast, Four-Color In Vivo Imaging with Shortwave Infrared Detection. J Am Chem Soc 143, 6836-6846 (2021).
59. J. A. Carr, M. Aellen, D. Franke, P. T. C. So, O. T. Bruns, M. G. Bawendi, Absorption by water increases fluorescence image contrast of biological tissue in the shortwave infrared. Proceedings of the National Academy of Sciences 115, 9080-9085 (2018).
60. N. V Gopee, D. W. Roberts, P. Webb, C. R. Cozart, P. H. Siitonen, A. R. Warbritton, W. W. Yu, V. L. Colvin, N. J. Walker, P. C. Howard, Migration of intradermally injected quantum dots to sentinel organs in mice. Toxicol Sci 98, 249-257 (2007).
61. H. Soo Choi, W. Liu, P. Misra, E. Tanaka, J. P. Zimmer, B. Itty Ipe, M. G. Bawendi, J. V Frangioni, Renal clearance of quantum dots. Nat Biotechnol 25, 1165-1170 (2007).
62. A. S. Machado, D. M. Darmohray, J. Fayad, H. G. Marques, M. R. Carey, A quantitative framework for whole-body coordination reveals specific deficits in freely walking ataxic mice. Elife 4 (2015).
63. R. Kawai, T. Markman, R. Poddar, R. Ko, A. L. Fantana, A. K. Dhawale, A. R. Kampff, B. P. Ölveczky, Motor Cortex Is Required for Learning but Not for Executing a Motor Skill. Neuron 86, 800-812 (2015).
64. H. Kobayashi, Y. Hama, Y. Koyama, T. Barrett, C. A. S. Regino, Y. Urano, P. L. Choyke, Simultaneous Multicolor Imaging of Five Different Lymphatic Basins Using Quantum Dots. Nano Lett 7, 1711-1716 (2007).
65. I. L. Medintz, H. T. Uyeda, E. R. Goldman, H. Mattoussi, Quantum dot bioconjugates for imaging, labelling and sensing. Nat Mater 4, 435-446 (2005).
66. Y. Xu, J. Zhang, Q. Zhang, D. Tao, ViTPose: Simple Vision Transformer Baselines for Human Pose Estimation. Adv Neural Inf Process Syst 35 (2022).
67. B. M. Zeglis, C. B. Davis, R. Aggeler, H. C. Kang, A. Chen, B. J. Agnew, J. S. Lewis, Enzyme-Mediated Methodology for the Site-Specific Radiolabeling of Antibodies Based on Catalyst-Free Click Chemistry. Bioconjug Chem 24, 1057-1067 (2013).
68. R. Hartley, A. Zisserman, Multiple View Geometry in Computer Vision (Cambridge University Press, 2004; https://www.cambridge.org/core/books/multiple-view-geometry-in-computer-vision/0B6F289C78B2B23F596CAA76D3D43F7A).
69. C. Stringer, T. Wang, M. Michaelos, M. Pachitariu, Cellpose: a generalist algorithm for cellular segmentation. Nat Methods 18, 100-106 (2021).

1. A method of measuring fluorescence in an animal, the method comprising (a) injecting the animal with one or more fluorescent particles; and (b) capturing the fluorescence exhibited by the animal; wherein the fluorescent particle is bound to a microparticle.

2. The method of claim 1, wherein the fluorescent particle is a quantum dot.

3. The method of claim 1, wherein the microparticle is a polymethyl-methacrylate (PMMA) bead, an agarose bead, or other microparticle coated with an antibody.

4. The method of claim 1, wherein the fluorescent particle is coated in biotin, and the microparticle is streptavidin.

5. The method of claim 3, wherein the antibody is an antibody-binding protein to collagen.

6. The method of claim 3, wherein the antibody is an antibody-binding protein to fibronectin.

7. The method of claim 2, wherein the quantum dot emits at an excitation from about 600 nm to about 1000 nm.

8. The method of claim 1, wherein the animal is a mammal.

9. The method of claim 8, wherein the mammal is a mouse.

10. The method of claim 9, wherein the mouse is injected in at least one of the right paw, the left paw, the right hind leg, the left hind leg, the tail, subdermal, the spine, joints (e.g. knee, wrist), the ears, the head and other internal organs (e.g. the bladder).

11. The method of claim 1, wherein a camera captures the fluorescence.

12. The method of claim 11, wherein the camera is a near infrared camera.

13. The method of claim 1, wherein the animal is a diseased animal, an animal that has been treated (e.g. with a drug), or a genetically-modified animal.

14. The method of claim 13, wherein the disease is a neurodegenerative disease.

15. The method of claim 14, wherein the neurodegenerative disease is Parkinson's or Huntington's.

16. A method of measuring movement of an animal, the method comprising:
(a) injecting the animal with one or more fluorescent particle(s) bound to a microparticle;
(b) capturing the fluorescence exhibited by the animal; and
(c) monitoring the fluorescence to measure the movement of the animal.

17. The method of claim 16, wherein the movement of the animal is measured using a camera.

18. The method of claim 16, wherein the animal has a neurodegenerative disorder.

19. The method of claim 16, wherein the method further comprises comparing the movement of the animal to a set of criteria to determine if the animal suffers from a neurodegenerative disorder; wherein the criteria is if the animal has at least one of a halting gait, a tremor, dyskinesia, tics, or chorea.

20. The method of claim 16, wherein the microparticle is a quantum dot.
